(19) United States
(12) Patent Application Publication
(10) Pub. No.: US 2018/0177784 A1
(43) Pub. Date: Jun. 28, 2018

(54) HETEROCYCLIC COMPOUNDS AS IMMUNOMODULATORS

(71) Applicant: Incyte Corporation, Wilmington, DE (US)

(72) Inventors: Liangxing Wu, Wilmington, DE (US); Ding-Quan Qian, Newark, DE (US); Liang Lu, Hockessin, DE (US); Neil Lajkiewicz, Garnet Valley, PA (US); Leah C. Konkol, Wilmington, DE (US); Zhenwu Li, Wilmington, DE (US); Fenglei Zhang, Berwyn, PA (US); Jingwei Li, Westfield, NJ (US); Haisheng Wang, Hockessin, DE (US); Meizhong Xu, Hockessin, DE (US); Kaijiong Xiao, Clark, NJ (US); Wenqing Yao, Chadds Ford, PA (US)

(21) Appl. No.: 15/851,280

(22) Filed: Dec. 21, 2017

Related U.S. Application Data
(60) Provisional application No. 62/437,998, filed on Dec. 22, 2016.
(60) Provisional application No. 62/487,365, filed on Apr. 19, 2017.

(51) Int. Cl. A61K 31/519; A61K 31/5025; A61K 31/4985; A61K 31/4725; A61K 31/4709; A61K 31/445; A61K 31/4375; A61K 31/437; A61K 31/4355; A61K 31/429; A61K 31/426; A61K 31/4196; A61K 31/4162; A61K 31/4155; A61K 31/421; A61K 31/404; A61K 31/357; A61K 31/353; A61K 31/133; A61P 31/00; A61P 35/00

(52) U.S. Cl. CPC A61K 31/519; A61K 2300/00; A61K 31/4985; A61K 31/4725; A61K 31/4709; A61K 31/445; A61K 31/4375; A61K 31/437; A61K 31/4355; A61K 31/429; A61K 31/426; A61K 31/4196; A61K 31/4162; A61K 31/4155; A61K 31/421; A61K 31/404; A61K 31/357; A61K 31/353; A61K 31/133; A61P 31/00; A61P 35/00; A61K 31/5025

(57) ABSTRACT

Disclosed are compounds of Formula (I′), methods of using the compounds as immunomodulators, and pharmaceutical compositions comprising such compounds. The compounds inhibit PD-1/PD-L1 interaction and are useful in treating, preventing or ameliorating diseases or disorders such as cancer or infections.

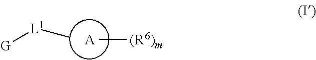

### FIELD OF THE INVENTION

The present application is concerned with pharmaceutically active compounds. The disclosure provides compounds as well as their compositions and methods of use. The compounds modulate PD-1/PD-L1 protein/protein interaction and are useful in the treatment of various diseases including infectious diseases and cancer.

### BACKGROUND OF THE INVENTION

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al, J. Clinical Oncology 2015, 1-9). Blocking the signaling of an inhibitory immune checkpoint, such as PD-1, has proven to be a promising and effective treatment modality.

Programmed cell death-1 (PD-1), also known as CD279, is a cell surface receptor expressed on activated T cells, natural killer T cells, B cells, and macrophages (Greenwald et al, Annu. Rev. Immunol 2005, 23:515-548; Okazaki and Honjo, Trends Immunol 2006, (4): 195-201). It functions as an intrinsic negative feedback system to prevent the activation of T-cells, which in turn reduces autoimmunity and promotes self-tolerance. In addition, PD-1 is also known to play a critical role in the suppression of antigen-specific T cell response in diseases like cancer and viral infection (Sharpe et al, Nat Immunol 2007 8, 239-245; Postow et al, J. Clinical Oncol 2015, 1-9).

The structure of PD-1 consists of an extracellular immunoglobulin variable-like domain followed by a transmembrane region and an intracellular domain (Parry et al, Mol Cell Biol 2005, 9543-9553). The intracellular domain contains two phosphorylation sites located in an immunoreceptor tyrosine-based inhibitory motif and an immunoreceptor tyrosine-based switch motif, which suggests that PD-1 negatively regulates T cell receptor-mediated signals. PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-γ treatment (Iwai et al, PNAS2002, 99(19):12293-7; Blank et al, Cancer Res 2004, 64(3):1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-γ production, as well as cell proliferation induced upon T cell receptor activation (Carter et al, Eur J Immunol 2002, 32(3):634-43; Freeman et al, J Exp Med 2000, 192(7): 1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and API pathways, and for cytokine production such as IL-2, IFN-γ and TNF (Sharpe et al, Nat Immunol 2007, 8, 239-245; Carter et al, Eur J Immunol 2002, 32(3):634-43; Freeman et al, J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:141-151; Nishimura et al, Science 2001, 291:319-322). Using an LCMV model of chronic infection, it has been shown that PD-1/PD-L1 interaction inhibits activation, expansion and acquisition of effector functions of virus-specific CD8 T cells (Barber et al, Nature 2006, 439, 682-7). Together, these data support the development of a therapeutic approach to block the PD-1-mediated inhibitory signaling cascade in order to augment or “rescue” T cell response. Accordingly, there is a need for new compounds that block PD-1/PD-L1 protein/protein interaction.

### SUMMARY

The present disclosure provides a compound of Formula (I′):

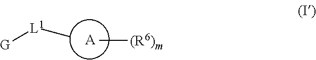

or a pharmaceutically acceptable salt or a stereoisomer thereof, wherein constituent variables are defined herein.

The present disclosure provides, inter alia, a compound of Formula (I):

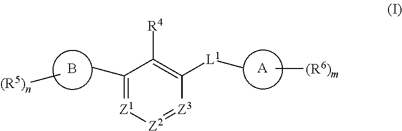

or a pharmaceutically acceptable salt or a stereoisomer thereof, wherein constituent variables are defined herein.

The present disclosure further provides a pharmaceutical composition comprising a compound disclosed herein, or a pharmaceutically acceptable salt or a stereoisomer thereof, and one or more pharmaceutically acceptable excipient or carrier.

The present disclosure further provides methods of inhibiting PD-1/PD-L1 interaction, said method comprising administering to a patient a compound disclosed herein, or a pharmaceutically acceptable salt or a stereoisomer thereof.

The present disclosure further provides methods of treating a disease or disorder associated with inhibition of PD-1/PD-L1 interaction, said method comprising administering to a patient in need thereof a therapeutically effective amount of a compound of disclosed herein, or a pharmaceutically acceptable salt or a stereoisomer thereof.

The present disclosure further provides methods of enhancing, stimulating and/or increasing the immune response in a patient, said method comprising administering to the patient in need thereof a therapeutically effective amount of a compound disclosed herein, or a pharmaceutically acceptable salt or a stereoisomer thereof.

### DETAILED DESCRIPTION

### I. Compounds

The present disclosure provides, inter alia, a compound of Formula (I′):

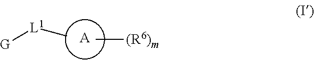

or a pharmaceutically acceptable salt or a stereoisomer thereof, wherein:

G has Formula (I′e) or (I′b)

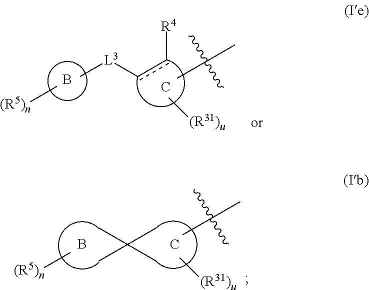

when G is of Formula (I′a), the atoms on ring C, to which the substituent R⁴ and ring B are attached can be either carbon or nitrogen; and [symbol] is a single bond or a double bond;

when G is of Formula (I′b), ring B and ring C are joined together through a quaternary ring carbon atom to form a spiro structure and ring B and ring C are each independently 4- to 14-membered heterocycloalkyl or C₃₋₁₄ cycloalkyl;

L¹ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, —(CR¹⁴R¹⁵)ₜC(═S)NR¹³(CR¹⁴R¹⁵)ₜ—, —(CR¹⁴R¹⁵)ₜNR¹³C(═S)(CR¹⁴R¹⁵)ₜ—, —(CR¹⁴R¹⁵)ₜC(═NR¹³)NR¹³(CR¹⁴R¹⁵)ₜ—, —(CR¹⁴R¹⁵)ₜNR¹³C(═NR¹³)(CR¹⁴R¹⁵)ₜ—, —(CR¹⁴R¹⁵)ₜC(═NOR¹³)NR¹³(CR¹⁴R¹⁵)ₜ—, —(CR¹⁴R¹⁵)ₜNR¹³C(═NOR¹³)(CR¹⁴R¹⁵)ₜ—, —(CR¹⁴R¹⁵)ₜC(═NCN)NR¹³(CR¹⁴R¹⁵)ₜ—, —(CR¹⁴R¹⁵)ₜNR¹³C(═NCN)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, S, —(CR¹⁴R¹⁵)ₚ—S—, —S(CR¹⁴R¹⁵)ₚ—, —(CR¹⁴R¹⁵)ₚ—S—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³SO₂NR¹³(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;

L³ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, —(CR¹⁴R¹⁵)ₜC(═S)NR¹³(CR¹⁴R¹⁵)ₜ—, —(CR¹⁴R¹⁵)ₜNR¹³C(═S)(CR¹⁴R¹⁵)ₜ—, —(CR¹⁴R¹⁵)ₜC(═NR³)NR¹³(CR¹⁴R¹⁵)ₜ—, —(CR¹⁴R¹⁵)ₜNR¹³C(═NR¹³)(CR¹⁴R¹⁵)ₜ—, —(CR¹⁴R¹⁵)ₜC(═NOR¹³)NR¹³(CR¹⁴R¹⁵)ₜ—, —(CR¹⁴R¹⁵)ₜNR¹³C(═NOR¹³)(CR¹⁴R¹⁵)ₜ—, —(CR¹⁴R¹⁵)ₜC(═NCN)NR¹³(CR¹⁴R¹⁵)ₜ—, —(CR¹⁴R¹⁵)ₜNR¹³C(═NCN)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, S, —(CR¹⁴R¹⁵)ₚ—S—, —S(CR¹⁴R¹⁵)ₚ—, —(CR¹⁴R¹⁵)ₚ—S—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³SO₂NR¹³(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;

ring A is C₆₋₁₀ aryl, 5- to 14-membered heteroaryl, 4- to 14-membered heterocycloalkyl or C₃₋₁₄ cycloalkyl;

ring B is C₆₋₁₀ aryl, 5- to 14-membered heteroaryl, 4- to 14-membered heterocycloalkyl or C₃₋₁₄ cycloalkyl;

ring C is C₆₋₁₀ aryl, 5- to 14-membered heteroaryl, 4- to 14-membered heterocycloalkyl or C₃₋₁₄ cycloalkyl;

each R¹³ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;

R¹⁴ and R¹⁵ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R¹⁴ or R¹⁵ are each optionally substituted with 1, 2, or 3 independently selected R^{q} substituents;

or R¹⁴ and R¹⁵ taken together with the carbon atom to which they are attached form C₃₋₆ cycloalkyl or 4- to 7-membered heterocycloalkyl, each of which is optionally substituted with 1, 2, or 3 independently selected R^{q} substituents;

R⁴ is H, halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl;

R⁵, R⁶ and R³¹ are each independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-(5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, C(O)NR^{a}S(O)₂R^{a}, NR^{a}C(═NR^{a})R^{a}, S(O)₂NR^{a}C(O)R^{a}, —P(O)R^{a}R^{a}, —P(O)(OR^{a})(OR^{a}), —B(OH)₂, —B(OR^{a})₂, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-(5-14 membered heteroaryl)-C₁₋₄ alkyl-, and (4-10 membered heterocycloalkyl)-C₁₋₄ alkyl- of R⁵, R⁶ and R³¹ are each optionally substituted with 1, 2, 3, 4 or 5 independently selected R^{b} substituents;

or two adjacent R⁵ substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, B, P, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R⁵ substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, B, P, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

or two adjacent R⁶ substituents on ring A, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, B, P, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R⁶ substituents on the same ring carbon atom of the ring A, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, B, P, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

or two adjacent R³¹ substituents on ring C, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, B, P, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R³¹ substituents on the same ring carbon atom of ring C, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, B, P, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2 or 3 independently selected R^{d} substituents;

each R^{b} substituent is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c}, C(O)NR^{c}S(O)₂R^{c}, NR^{c}C(═NR^{c})R^{c}, S(O)₂NR^{c}C(O)R^{c}, —P(O)R^{c}R^{c}, —P(O)(OR^{c})(OR^{c}), —B(OH)₂, —B(OR^{c})₂, and S(O)₂NR^{c}R^{c}; wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1, 2, or 3 independently selected R^{d} substituents;

or two R^{b} substituents attached to the same ring carbon atom taken together with the ring carbon atom to which they are attached form spiro C₃₋₆ cycloalkyl or spiro 4- to 7-membered heterocycloalkyl, each of which is optionally substituted with 1, 2, or 3 independently selected R^{f} substituents;

each R^{c} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2 or 3 independently selected R^{f} substituents;

each R^{f} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, C(O)NR^{g}S(O)₂R^{g}, NR^{g}C(═NR^{g})R^{g}, S(O)₂NR^{g}C(O)R^{g}, —P(O)R^{g}R^{g}, —P(O)(OR^{g})(OR^{g}), —B(OH)₂, —B(OR^{g})₂, and S(O)₂NR^{g}R^{g}; wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2 or 3 independently selected Rⁿ substituents;

each Rⁿ is independently selected from C₁₋₆ alkyl, C₁₋₆ haloalkyl, halo, CN, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, C(O)NR^{o}S(O)₂R^{o}, NR^{o}C(═NR^{o})R^{o}, S(O)₂NR^{o}C(O)R^{o}, —P(O)R^{o}R^{o}, —P(O)(OR^{o})(OR^{o}), —B(OH)₂, —B(OR^{o})₂, and S(O)₂NR^{o}R^{o}, wherein the C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of Rⁿ is optionally substituted with 1, 2 or 3 independently selected R^{q} substituents;

each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, C(O)NR^{e}S(O)₂R^{e}, NR^{e}C(═NR^{e})R^{e}, S(O)₂NR^{e}C(O)R^{e}, —P(O)R^{e}R^{e}, —P(O)(OR^{e})(OR^{e}), —B(OH)₂, —B(OR^{e})₂, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1, 2, or 3 independently selected R^{f} substituents;

each R^{e} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{e} are each optionally substituted with 1, 2 or 3 independently selected R^{f} substituents; each R^{g} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1, 2, or 3 independently selected R^{p} substituents;

each R^{p} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r}, C(O)NR^{r}S(O)₂R^{r}, NR^{r}C(═NR^{r})R^{r}, S(O)₂NR^{r}C(O)R^{r}, —P(O)R^{r}R^{r}, —P(O)(OR^{r})(OR^{r}), —B(OH)₂, —B(OR^{r})₂, and S(O)₂NR^{r}R^{r}, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} is optionally substituted with 1, 2 or 3 independently selected R^{q} substituents;

or any two R^{a} substituents together with the nitrogen atom to which they are attached form a 4-, 5-, 6-, 7-, 8-, 9- or 10-membered heterocycloalkyl group optionally substituted with 1, 2 or 3 independently selected R^{h} substituents;

each R^{h} is independently selected from C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, C₆₋₁₀ aryl-C₁₋₄ alkyl-, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, NRⁱC(═NOH)NRⁱRⁱ, NRⁱC(═NCN)NRⁱRⁱ, S(O)Rⁱ, S(O)NRⁱRⁱ, S(O)₂Rⁱ, NRⁱS(O)₂Rⁱ, NRⁱS(O)₂NRⁱRⁱ, C(O)NRⁱS(O)₂Rⁱ, NRⁱC(═NRⁱ)Rⁱ, S(O)₂NRⁱC(O)Rⁱ, P(O)RⁱRⁱ, —P(O)(ORⁱ)(ORⁱ), —B(OH)₂, —B(ORⁱ)₂, and S(O)₂NRⁱRⁱ, wherein the C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{h} are each optionally substituted by 1, 2, or 3 independently selected R^{j} substituents;

each R^{j} is independently selected from C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₂₋₄ alkynyl, halo, C₁₋₄ haloalkyl, C₁₋₄ haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR^{k})NR^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, C(O)NR^{k}S(O)₂R^{k}, NR^{k}C(═NR^{k})R^{k}, S(O)₂NR^{k}C(O)R^{k}, P(O)R^{k}R^{k}, —P(O)(OR^{k})(OR^{k}), —B(OH)₂, —B(OR^{k})₂, and S(O)₂NR^{k}R^{k}, wherein the C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₁₋₄ haloalkyl, and C₁₋₄ haloalkoxy of R^{j} are each optionally substituted with 1, 2 or 3 independently selected R^{q} substituents;

or two R^{h} groups attached to the same carbon atom of the 4- to 10-membered heterocycloalkyl, taken together with the carbon atom to which they are attached, form a C₃₋₆ cycloalkyl or 4- to 6-membered heterocycloalkyl having 1-2 heteroatoms as ring members selected from O, N or S;

or any two R^{c} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two R^{e} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two R^{g} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two Rⁱ substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents, or 1, 2, or 3 independently selected R^{q} substituents;

or any two R^{k} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents, or 1, 2, or 3 independently selected R^{q} substituents;

or any two R^{o} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents; and

or any two R^{r} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

each Rⁱ, R^{k}, R^{o} or R^{r} is independently selected from H, C₁₋₆ alkyl, C₁₋₆ haloalkyl, C₃₋₆ cycloalkyl, C₆₋₁₀ aryl, 4-6 membered heterocycloalkyl, 5 or 6-membered heteroaryl, C₁₋₄ haloalkyl, C₂₋₄ alkenyl, and C₂₋₄ alkynyl, wherein the C₁₋₄ alkyl, C₁₋₆ haloalkyl, C₃₋₆ cycloalkyl, C₆₋₁₀ aryl, 4-6 membered heterocycloalkyl, 5 or 6-membered heteroaryl, C₂₋₄ alkenyl, and C₂₋₄ alkynyl of Rⁱ, R^{k}, R^{o} or R^{r} are each optionally substituted with 1, 2 or 3 R^{q} substituents;

each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NHR¹² and NR¹²R¹², wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl and 4-6 membered heterocycloalkyl and each R¹² is independently C₁₋₆ alkyl;

the subscript n is an integer of 0, 1, 2, 3, 4, 5, 6, 7 or 8;

the subscript m is an integer of 0, 1, 2, 3, 4, 5, 6, 7 or 8;

each subscript p is independently an integer of 1, 2, 3 or 4;

each subscript t is independently an integer of 0, 1, 2, 3 or 4;

the subscript u is an integer of 0, 1, 2, 3, 4, 5, 6, 7 or 8;

with the provisos:

(i) when L¹ is a bond and

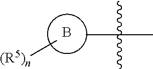

is phenyl or 2,3-dihydro-1,4-benzodioxin-6-yl, then

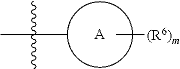

is not

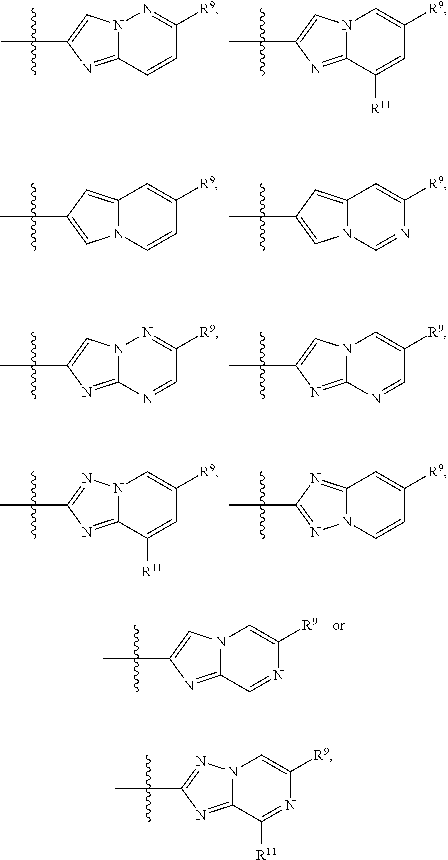

wherein each R⁹ is independently (2-hydroxyethylamino)methyl or (2-carboxy-1-piperidinyl)methyl and each R¹¹ is independently H, halo, CN, C₁₋₆ alkyl, C₁₋₆ alkoxy, —NHC₁₋₆ alkyl or benzyloxy, wherein the C₁₋₆ alkyl, C₁₋₆ alkoxy, —NHC₁₋₆ alkyl and benzyloxy of R¹¹ are each optionally substituted with halo, CN, C₁₋₆ alkyl or C₁₋₆ alkoxy;

(ii) when L¹ is a bond and

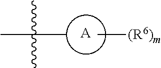

is phenyl or 2,3-dihydro-1,4-benzodioxin-6-yl, then

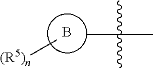

is not any of the moieties set forth in proviso (i) above for

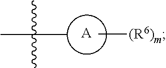

(iii) when L¹ is a bond, then

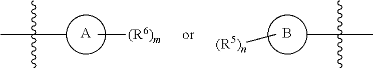

is not

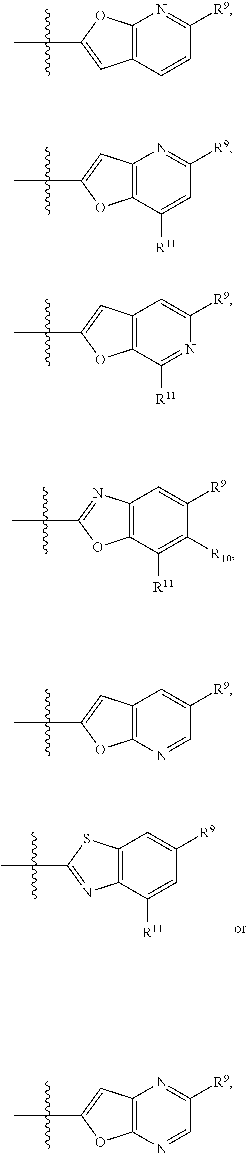

wherein each R⁹ is independently (2-hydroxyethylamino)methyl or (2-carboxy-1-piperidinyl)methyl; each R¹¹ is independently H or C₁₋₆ alkyl and R¹⁰ is H, C₁₋₆ alkoxy, benzyloxy, morpholinoethoxy or 2-pyridylmethyloxy, wherein the C₁₋₆ alkoxy, benzyloxy and 2-pyridylmethyloxy of R¹⁰ are each optionally substituted with CN;

(iv) when L¹ is a bond, then

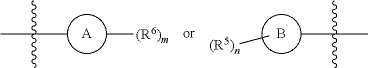

is not

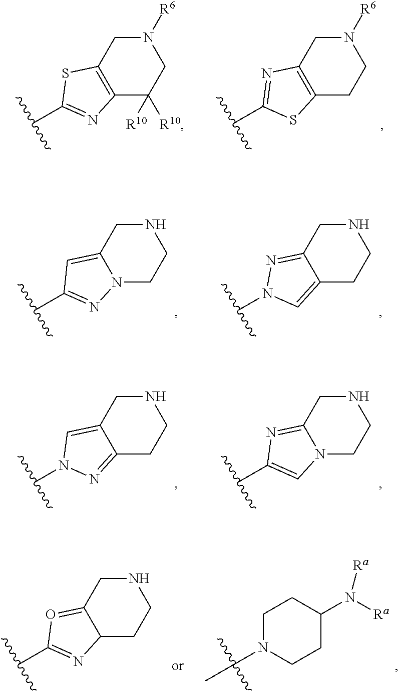

wherein R¹⁰ is H or C₁₋₆ alkyl;

(v) when L¹ is —NHC(O)—, then

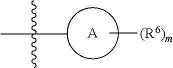

is not

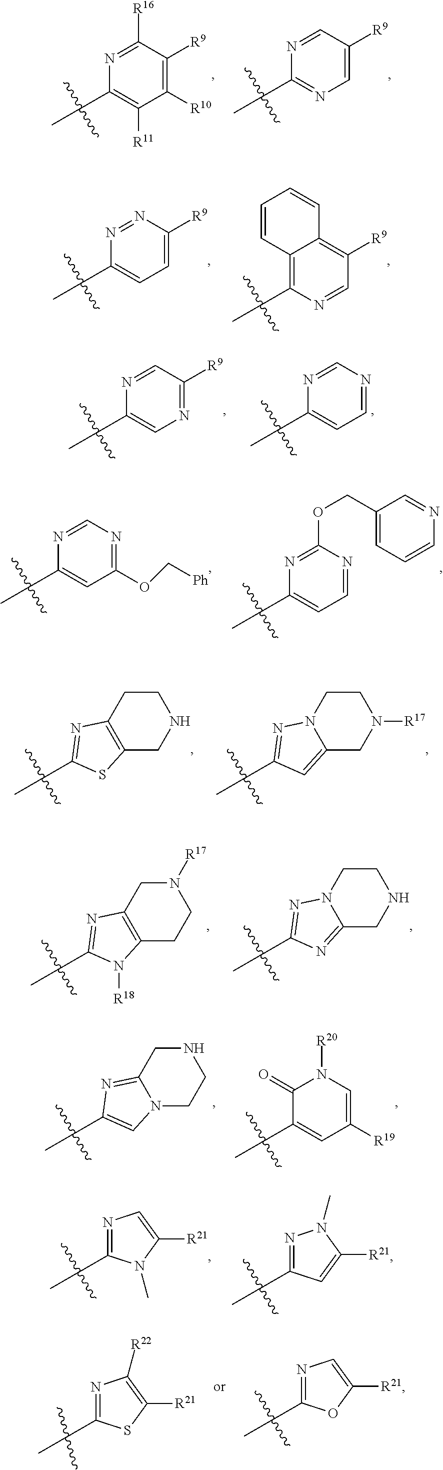

wherein each R⁹ is independently H, methyl, (2-hydroxyethylamino)methyl or (2-carboxy-1-piperidinyl)methyl; R¹⁰ is H, methyl, CN, methoxy, cyclopropylmethoxy, benzyloxy, (2-cyanophenyl)methoxy, 2-pyridylmethoxy, 3-pyridylmethoxy, (2-hydroxyethylamino)methyl or (2-carboxy-1-piperidinyl)methyl; R¹¹ is H, halo, methyl or dimethylamino; R¹⁶ is H or methyl; each R¹⁷ is independently H, 2-hydroxyethyl or carboxymethyl; R¹⁸ is H or methyl; R¹⁹ is (2-hydroxyethylamino)methyl or (2-carboxy-1-piperidinyl)methyl; R²⁰ is C₁₋₆ alkyl; each R²¹ is independently 2-hydroxyethylamino)methyl or (2-carboxy-1-piperidinyl)methyl; and R²² is H or Cl;

(vi) when L¹ is —NH— and

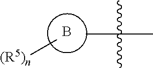

is phenyl, 2,3-dihydro-1,4-benzodioxin-6-yl, cyclohexyl or 1-cyclohexenyl, then

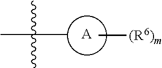

is not

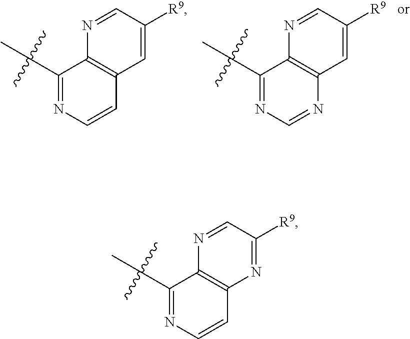

wherein each R⁹ is independently (2-hydroxyethylamino)methyl or (2-carboxy-1-piperidinyl)methyl;

(vii) when L¹ is —CH₂O—, ring B is phenyl or thienyl, and the subscript n is 1 or 2, then R⁵ is not a substituent independently selected from H, —OCH₃, —OH, —OCH₂CH₃, —O(CH₂)OCH₃, —OCH₂CH═CH₂, —O(CH₂)₂CH₃, —O(CH₂)₂morpholinyl or F; and

(viii) when L¹ is —CH₂O—, ring B is phenyl or thienyl, and the subscript n is 2, then two R⁵ substituents attached to adjacent ring carbon atoms of ring B do not form —OCH₂O— or —OCH₂CH₂O—; and

wherein the compound, or a pharmaceutically acceptable salt or a stereoisomer thereof inhibits PD-1/PD-L1 interaction.

In some embodiments, G has Formula (I′a) or (I′b):

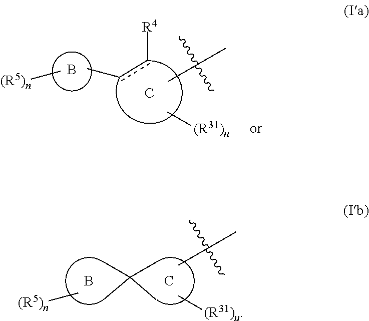

In some embodiments,

when ring C is 4- to 14-membered heterocycloalkyl or C₃₋₁₄ cycloalkyl,

R⁴ is H, halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl.

In some embodiments, R⁴ is halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸;

wherein each R⁸ is independently H or C₁₋₆ alkyl.

In some embodiments of the compound of Formula (I′):

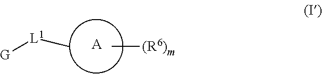

or a pharmaceutically acceptable salt or a stereoisomer thereof,

G has Formula (I′a) or (I′b)

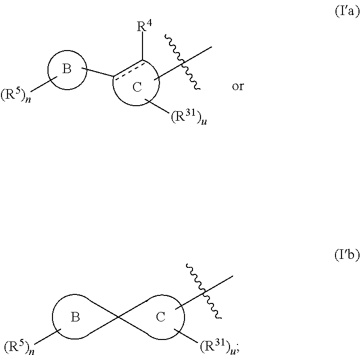

when G is of Formula (I′a), the atoms on ring C, to which the substituent R⁴ and ring B are attached can be either carbon or nitrogen; and [symbol] is a single bond or a double bond;

when G is of Formula (I′b), ring B and ring C are joined together through a quaternary ring carbon atom to form a spiro structure and ring B and ring C are each independently 4- to 14-membered heterocycloalkyl or C₃₋₁₄ cycloalkyl;

L¹ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³SO₂NR¹³(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;

ring A is C₆₋₁₀ aryl, 5- to 14-membered heteroaryl, 4- to 14-membered heterocycloalkyl or C₃₋₁₄ cycloalkyl;

ring B is C₆₋₁₀ aryl, 5- to 14-membered heteroaryl, 4- to 14-membered heterocycloalkyl or C₃₋₁₄ cycloalkyl;

ring C is C₆₋₁₀ aryl, 5- to 14-membered heteroaryl, 4- to 14-membered heterocycloalkyl or C₃₋₁₄ cycloalkyl;

each R¹³ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;

R¹⁴ and R¹⁵ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R¹⁴ or R¹⁵ are each optionally substituted with 1, 2, or 3 independently selected R^{q} substituents;

or R¹⁴ and R¹⁵ taken together with the carbon atom to which they are attached form C₃₋₆ cycloalkyl or 4- to 7-membered heterocycloalkyl, each of which is optionally substituted with 1, 2, or 3 independently selected R^{q} substituents;

R⁴ is halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl;

R⁵, R⁶ and R³¹ are each independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-(5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R⁵, R⁶ and R³¹ are each optionally substituted with 1, 2, 3, 4 or 5 independently selected R^{b} substituents;

or two adjacent R⁵ substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R⁵ substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

or two adjacent R⁶ substituents on ring A, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R⁶ substituents on the same ring carbon atom of the ring A, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

or two adjacent R³¹ substituents on ring C, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R³¹ substituents on the same ring carbon atom of ring C, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2 or 3 independently selected R^{d} substituents;

each R^{b} substituent is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1, 2, or 3 independently selected R^{d} substituents;

or two R^{b} substituents attached to the same ring carbon atom taken together with the ring carbon atom to which they are attached form spiro C₃₋₆ cycloalkyl or spiro 4- to 7-membered heterocycloalkyl, each of which is optionally substituted with 1, 2, or 3 independently selected R^{f} substituents;

each R^{c} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2 or 3 independently selected R^{f} substituents;

each R^{f} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2 or 3 independently selected Rⁿ substituents;

each R¹¹ is independently selected from C₁₋₆ alkyl, C₁₋₆ haloalkyl, halo, CN, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o}, wherein the C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of Rⁿ is optionally substituted with 1, 2 or 3 independently selected R^{q} substituents;

each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1, 2, or 3 independently selected R^{f} substituents;

each R^{e} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{e} are each optionally substituted with 1, 2 or 3 independently selected R^{f} substituents;

each R^{g} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1, 2, or 3 independently selected R^{p} substituents;

each R^{p} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r} and S(O)₂NR^{r}R^{r}, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} is optionally substituted with 1, 2 or 3 independently selected R^{q} substituents;

or any two R^{a} substituents together with the nitrogen atom to which they are attached form a 4-, 5-, 6-, 7-, 8-, 9- or 10-membered heterocycloalkyl group optionally substituted with 1, 2 or 3 independently selected R^{h} substituents;

each R^{h} is independently selected from C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, C₆₋₁₀ aryl-C₁₋₄ alkyl-, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, NRⁱC(═NOH)NRⁱRⁱ, NRⁱC(═NCN)NRⁱRⁱ, S(O)Rⁱ, S(O)NRⁱRⁱ, S(O)₂Rⁱ, NRⁱS(O)₂Rⁱ, NRⁱS(O)₂NRⁱ, and S(O)₂NRⁱRⁱ, wherein the C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{h} are each optionally substituted by 1, 2, or 3 independently selected R^{j} substituents;

each R^{j} is independently selected from C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₂₋₄ alkynyl, halo, C₁₋₄ haloalkyl, C₁₋₄ haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR^{k})NR^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}, wherein the C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₁₋₄ haloalkyl, and C₁₋₄ haloalkoxy of R^{j} are each optionally substituted with 1, 2 or 3 independently selected R^{q} substituents;

or two R^{h} groups attached to the same carbon atom of the 4- to 10-membered heterocycloalkyl, taken together with the carbon atom to which they are attached, form a C₃₋₆ cycloalkyl or 4- to 6-membered heterocycloalkyl having 1-2 heteroatoms as ring members selected from O, N or S;

or any two R^{c} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two R^{e} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two R^{g} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two Rⁱ substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two R^{k} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

or any two R^{o} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents; and

or any two R^{r} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;

each Rⁱ, R^{k}, R^{o} or R^{r} is independently selected from H, C₁₋₆ alkyl, C₁₋₆ haloalkyl, C₃₋₆ cycloalkyl, C₆₋₁₀ aryl, 4-6 membered heterocycloalkyl, 5 or 6-membered heteroaryl, C₁₋₄ haloalkyl, C₂₋₄ alkenyl, and C₂₋₄ alkynyl, wherein the C₁₋₄ alkyl, C₁₋₆ haloalkyl, C₃₋₆ cycloalkyl, C₆₋₁₀ aryl, 4-6 membered heterocycloalkyl, 5 or 6-membered heteroaryl, C₂₋₄ alkenyl, and C₂₋₄ alkynyl of Rⁱ, R^{k}, R^{o} or R^{r} are each optionally substituted with 1, 2 or 3 R^{q} substituents;

each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NHR¹² and NR¹²R¹², wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl and 4-6 membered heterocycloalkyl and each R¹² is independently C₁₋₆ alkyl;

the subscript n is an integer of 0, 1, 2, 3, 4, 5, 6, 7 or 8;

the subscript m is an integer of 0, 1, 2, 3, 4, 5, 6, 7 or 8;

each subscript p is independently an integer of 1, 2, 3 or 4;

each subscript t is independently an integer of 0, 1, 2, 3 or 4;

the subscript u is an integer of 0, 1, 2, 3, 4, 5, 6, 7 or 8;

with the provisos:

(i) when L¹ is a bond and

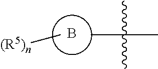

is phenyl or 2,3-dihydro-1,4-benzodioxin-6-yl, then

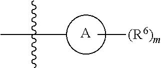

is not

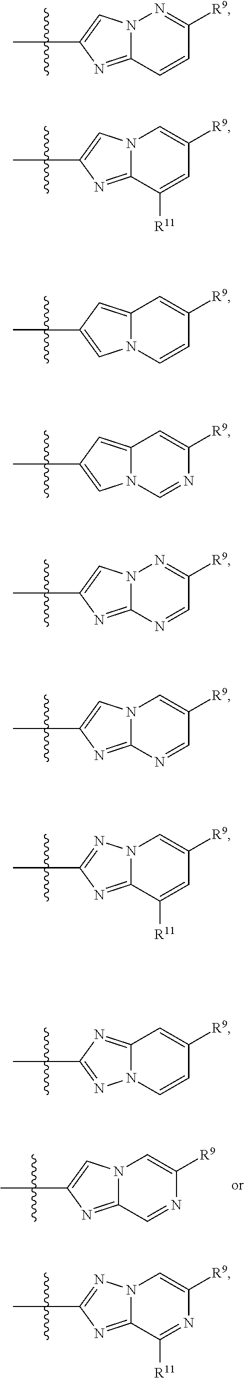

wherein each R⁹ is independently (2-hydroxyethylamino)methyl or (2-carboxy-1-piperidinyl)methyl and each R¹¹ is independently H, halo, CN, C₁₋₆ alkyl, C₁₋₆ alkoxy, —NHC₁₋₆ alkyl or benzyloxy, wherein the C₁₋₆ alkyl, C₁₋₆ alkoxy, —NHC₁₋₆ alkyl and benzyloxy of R¹¹ are each optionally substituted with halo, CN, C₁₋₆ alkyl or C₁₋₆ alkoxy;

(ii) when L¹ is a bond and

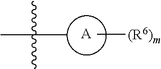

is phenyl or 2,3-dihydro-1,4-benzodioxin-6-yl, then

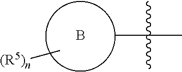

is not any of the moieties set forth in proviso (i) above for

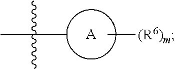

(iii) when L¹ is a bond, then

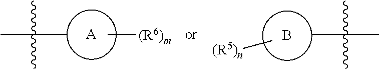

is not

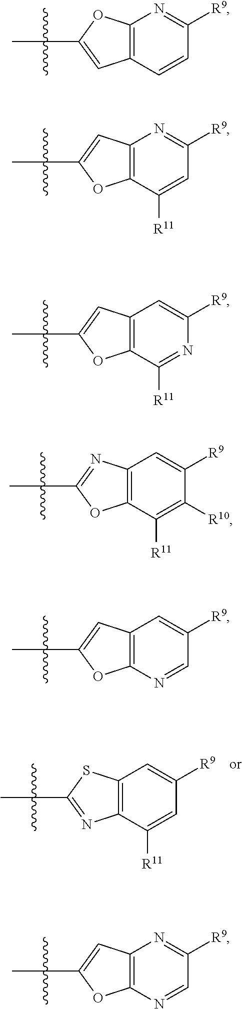

wherein each R⁹ is independently (2-hydroxyethylamino)methyl or (2-carboxy-1-piperidinyl)methyl; each R¹¹ is independently H or C₁₋₆ alkyl and R¹⁰ is H, C₁₋₆ alkoxy, benzyloxy, morpholinoethoxy or 2-pyridylmethyloxy, wherein the C₁₋₆ alkoxy, benzyloxy and 2-pyridylmethyloxy of R¹⁰ are each optionally substituted with CN;

(iv) when L¹ is a bond, then

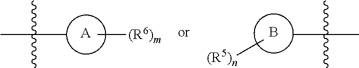

is not

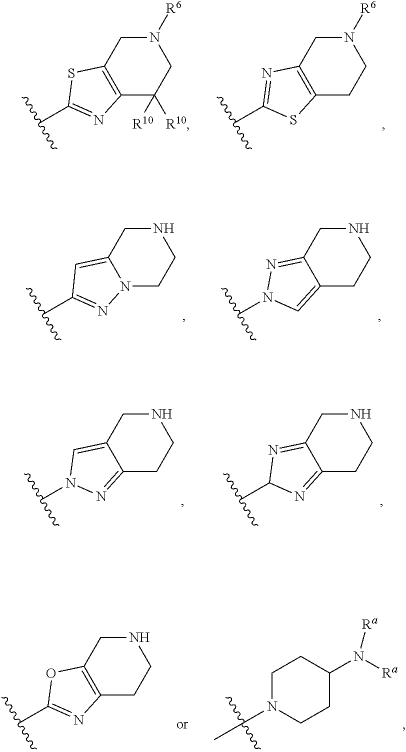

wherein R¹⁰ is H or C₁₋₆ alkyl;

(v) when L¹ is —NHC(O)—, then

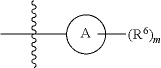

is not

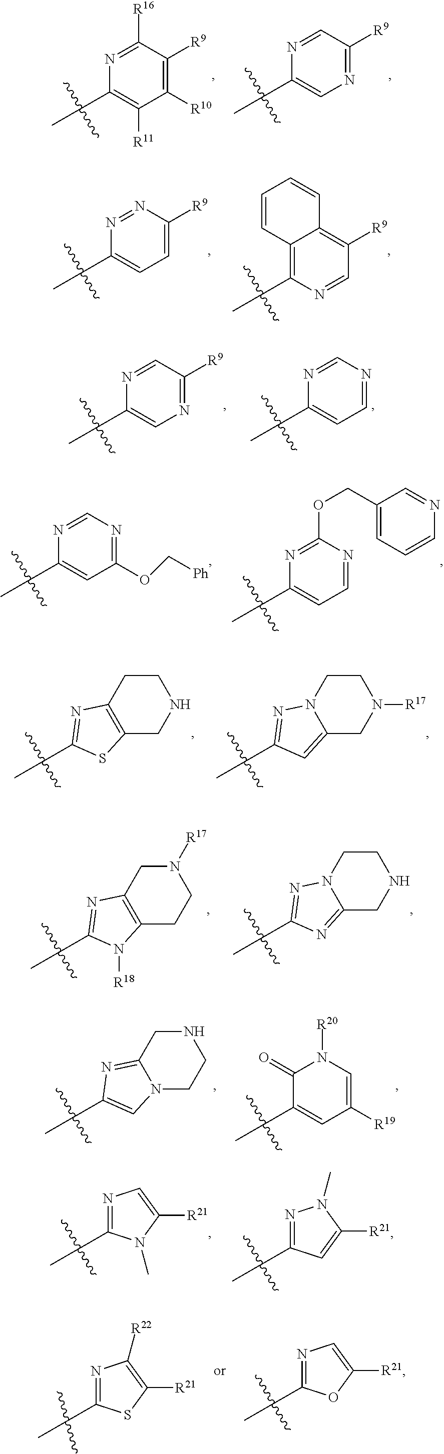

wherein each R⁹ is independently H, methyl, (2-hydroxyethylamino)methyl or (2-carboxy-1-piperidinyl)methyl; R¹⁰ is H, methyl, CN, methoxy, cyclopropylmethoxy, benzyloxy, (2-cyanophenyl)methoxy, 2-pyridylmethoxy, 3-pyridylmethoxy, (2-hydroxyethylamino)methyl or (2-carboxy-1-piperidinyl)methyl; R¹¹ is H, halo, methyl or dimethylamino; R¹⁶ is H or methyl; each R¹⁷ is independently H, 2-hydroxyethyl or carboxymethyl; R¹⁸ is H or methyl; R¹⁹ is (2-hydroxyethylamino)methyl or (2-carboxy-1-piperidinyl)methyl; R²⁰ is C₁₋₆ alkyl; each R²¹ is independently 2-hydroxyethylamino)methyl or (2-carboxy-1-piperidinyl)methyl; and R²² is H or Cl;

(vi) when L¹ is —NH— and

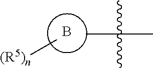

is phenyl, 2,3-dihydro-1,4-benzodioxin-6-yl, cyclohexyl or 1-cyclohexenyl, then

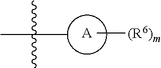

is not

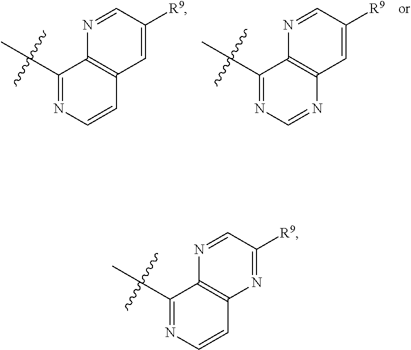

wherein each R⁹ is independently (2-hydroxyethylamino)methyl or (2-carboxy-1-piperidinyl)methyl;

(vii) when L¹ is —CH₂O—, ring B is phenyl or thienyl, and the subscript n is 1 or 2, then R⁵ is not a substituent independently selected from H, —OCH₃, —OH, —OCH₂CH₃, —O(CH₂)OCH₃, —OCH₂CH═CH₂, —O(CH₂)₂CH₃, —O(CH₂)₂morpholinyl or F; and

(viii) when L¹ is —CH₂O—, ring B is phenyl or thienyl, and the subscript n is 2, then two R⁵ substituents attached to adjacent ring carbon atoms of ring B do not form —OCH₂O— or —OCH₂CH₂O—; and

wherein the compound, or a pharmaceutically acceptable salt or a stereoisomer thereof inhibits PD-1/PD-L1 interaction.

In some embodiments, the present disclosure provides a compound of Formula (I′c):

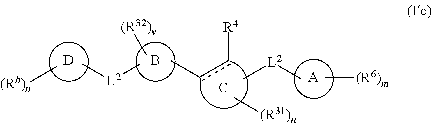

or a pharmaceutically acceptable salt or a stereoisomer thereof, wherein:

ring D is C₆₋₁₀ aryl, 5- to 14-membered heteroaryl, 4- to 14-membered heterocycloalkyl or C₃₋₁₄ cycloalkyl;

L² is a bond, —(CR²⁹R³⁰)ₜC(O)NR²⁸(CR²⁹R³⁰)ₜ—, —(CR²⁹R³⁰)ₜNR²⁸C(O)(CR²⁹R³⁰)ₜ—, O, —(CR²⁹R³⁰)_{q}—, —(CR²⁹R³⁰)_{q}—O—, —O(CR²⁹R³⁰)_{q}—, —(CR²⁹R³⁰)_{q}—O—(CR²⁹R³⁰)_{q}—, —NR²⁸—, —(CR²⁹R³⁰)_{w}NR²⁸(CR²⁹R³⁰)_{w}—, —NH—, —(CR²⁹R³⁰)_{w}NH(CR²⁹R³⁰)_{w}—, —CR²⁸═CR²⁸—, —C≡C—, —SO₂—, —(CR²⁹R³⁰)_{w}SO₂(CR²⁹R³⁰))_{w}—, —(CR²⁹R³⁰)_{w}SO₂NR²⁸(CR²⁹R³⁰)_{w}—, —(CR²⁹R³⁰)_{w}NR²⁸SO₂(CR²⁹R³⁰)_{w}—, —(CR²⁹R³⁰)_{w}NR²⁸SO₂NR²⁸(CR²⁹R³⁰)_{w}—, —(CR²⁹R³⁰)_{w}NR²⁸C(O)O(CR²⁹R³⁰)_{w}—, —NR²⁸C(O)O—, —(CR²⁹R³⁰)_{w}O(CO)NR²⁸(CR²⁹R³⁰)_{w}—, —O(CO)NR²⁸—, —NR²⁸C(O)NR²⁸— or —(CR²⁹R³⁰)_{w}NR²⁸C(O)NR²⁸(CR²⁹R³⁰)_{w};

each R²⁸ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;

R²⁹ and R³⁰ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R²⁹ or R³⁰ are each optionally substituted with 1, 2 or 3 independently selected R^{q} substituents;

or R²⁹ and R³⁰ taken together with the carbon atom to which they are attached form C₃₋₆ cycloalkyl or 4- to 7-membered heterocycloalkyl, each of which is optionally substituted with 1, 2, or 3 independently selected R^{q} substituents;

each R³² is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R³² are each optionally substituted with 1, 2, 3, 4 or 5 independently selected R^{b} substituents;

or two adjacent R³² substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents; the subscript n is an integer of 0, 1, 2, 3, 4, 5;

the subscript v is an integer of 0, 1, 2, 3, 4, 5, 6 or 7

each subscript q is independently an integer of 1, 2, 3 or 4;

each subscript t is independently an integer of 0, 1, 2, 3 or 4; and

each subscript w is independently an integer of 0, 1, 2, 3 or 4.

In some embodiments, the present disclosure provides a compound of Formula (I′d):

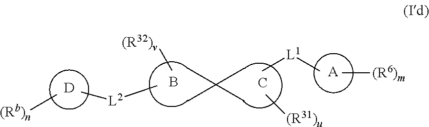

or a pharmaceutically acceptable salt or a stereoisomer thereof, wherein:

ring B and ring C are each independently 4- to 14-membered heterocycloalkyl or C₃₋₁₄ cycloalkyl;

ring D is C₆₋₁₀ aryl, 5- to 14-membered heteroaryl, 4- to 14-membered heterocycloalkyl or C₃₋₁₄ cycloalkyl;

L² is a bond, —(CR²⁹R³⁰)ₜC(O)NR²⁸(CR²⁹R³⁰)ₜ—, —(CR²⁹R³⁰)ₜNR²⁸C(O)(CR²⁹R³⁰)ₜ—, O, —(CR²⁹R³⁰)_{q}—, —(CR²⁹R³⁰)_{q}—O—, —O(CR²⁹R³⁰)_{q}—, —(CR²⁹R³⁰)_{q}—O—(CR²⁹R³⁰)_{q}—, —NR²⁸—, —(CR²⁹R³⁰)_{w}NR²⁸(CR²⁹R³⁰)_{w}—, —NH—, —(CR²⁹R³⁰)_{w}NH(CR²⁹R³⁰)_{w}—, —CR²⁸═CR²⁸—, —C≡C—, —SO₂—, —(CR²⁹R³⁰)_{w}SO₂(CR²⁹R³⁰)_{w}—, —(CR²⁹R³⁰)_{w}SO₂NR²⁸(CR²⁹R³⁰)_{w}—, —(CR²⁹R³⁰)_{w}NR²⁸SO₂(CR²⁹R³⁰)_{w}—, —(CR²⁹R³⁰)_{w}NR²⁸SO₂NR²⁸(CR²⁹R³⁰)_{w}—, —(CR²⁹R³⁰)_{w}NR²⁸C(O)O(CR²⁹R³⁰)_{w}—, —NR²⁸C(O)O—, —(CR²⁹R³⁰)_{w}O(CO)NR²⁸(CR²⁹R³⁰)_{w}—, —O(CO)NR²⁸—, —NR²⁸C(O)NR²⁸— or —(CR²⁹R³⁰)_{w}NR²⁸C(O)NR²⁸(CR²⁹R³⁰)_{w};

each R²⁸ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;

R²⁹ and R³⁰ are each independently selected from H, halo, CN, OH, NH₂, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R²⁹ or R³⁰ are each optionally substituted with 1, 2 or 3 independently selected R^{q} substituents;

or R²⁹ and R³⁰ taken together with the carbon atom to which they are attached form spiro C₃₋₆ cycloalkyl or spiro 4- to 7-membered heterocycloalkyl, each of which is optionally substituted with 1, 2, or 3 independently selected R^{q} substituents;

each R³² is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R³² are each optionally substituted with 1, 2, 3, 4 or 5 independently selected R^{b} substituents;

or two adjacent R³² substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R³² substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

the subscript n is an integer of 0, 1, 2, 3, 4, 5;

the subscript v is an integer of 0, 1, 2, 3, 4, 5, 6 or 7

each subscript q is independently an integer of 1, 2, 3 or 4;

each subscript t is independently an integer of 0, 1, 2, 3 or 4; and

each subscript w is independently an integer of 0, 1, 2, 3 or 4.

In some embodiments, the present disclosure provides a compound of Formula (I):

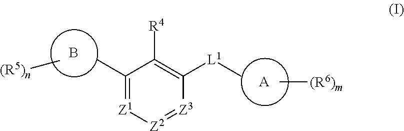

or a pharmaceutically acceptable salt or a stereoisomer thereof, wherein:

Z¹ is N or CR¹;

Z² is N or CR²;

Z³ is N or CR³;

L¹ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;

ring A is C₆₋₁₀ aryl, 5- to 14-membered heteroaryl, 4- to 11-membered heterocycloalkyl or C₃₋₁₀ cycloalkyl;

ring B is C₆₋₁₀ aryl, 5- to 14-membered heteroaryl, 4- to 11-membered heterocycloalkyl or C₃₋₁₀ cycloalkyl;

each R¹³ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;

R¹⁴ and R¹⁵ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R¹⁴ or R¹⁵ are each optionally substituted with 1, 2, or 3 independently selected R^{q} substituents;

or R¹⁴ and R¹⁵ taken together with the carbon atom to which they are attached form spiro C₃₋₆ cycloalkyl or spiro 4- to 7-membered heterocycloalkyl, each of which is optionally substituted with 1, 2, or 3 independently selected R^{q} substituents;

R¹, R² and R³ are each independently selected from H, C₁₋₄ alkyl, C₃₋₁₀ cycloalkyl, C₃₋₁₀ cycloalkyl-C₁₋₄ alkyl-, C₆₋₁₀ aryl, C₆₋₁₀ aryl-C₁₋₄ alkyl-, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, C₂₋₄ alkenyl, C₂₋₄ alkynyl, halo, CN, OR⁷, C₁₋₄ haloalkyl, C₁₋₄ haloalkoxy, NH₂, —NHR⁷, —NR⁷R⁷, NHOR⁷, C(O)R⁷, C(O)NR⁷R⁷, C(O)OR⁷, OC(O)R⁷, OC(O)NR⁷R⁷, NR⁷C(O)R⁷, NR⁷C(O)OR⁷, NR⁷C(O)NR⁷R⁷, C(═NR⁷)R⁷, C(═NR⁷)NR⁷R⁷, NR⁷C(═NR⁷)NR⁷R⁷, NR⁷S(O)R⁷, NR⁷S(O)₂R⁷, NR⁷S(O)₂NR⁷R⁷, S(O)R⁷, S(O)NR⁷R⁷, S(O)₂R⁷, and S(O)₂NR⁷R⁷, wherein each R⁷ is independently selected from H, C₁₋₄ alkyl, C₂₋₄ alkenyl, C₂₋₄ alkynyl, C₁₋₄ alkoxy, C₃₋₆ cycloalkyl, C₃₋₁₀ cycloalkyl-C₁₋₄ alkyl-, C₆₋₁₀ aryl, C₆₋₁₀ aryl-C₁₋₄ alkyl, 5-10 membered heteroaryl, 4-10 membered heterocycloalkyl, (5-10 membered heteroaryl)-C₁₋₄ alkyl-, and (4-10 membered heterocycloalkyl)-C₁₋₄ alkyl-, wherein the C₁₋₄ alkyl, C₁₋₄ alkoxy, C₃₋₁₀ cycloalkyl, C₃₋₆ cycloalkyl-C₁₋₄ alkyl-, C₆₋₁₀ aryl, C₆₋₁₀ aryl-C₁₋₄ alkyl-, 5-10 membered heteroaryl, 4-10 membered heterocycloalkyl, (5-10 membered heteroaryl)-C₁₋₄ alkyl-, and (4-10 membered heterocycloalkyl)-C₁₋₄ alkyl- of R¹, R², R³ and R⁷ are each optionally substituted with 1 or 2 independently selected R^{d} substituents;

R⁴ is halo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl or C₃₋₆ cycloalkyl, wherein the C₁₋₆ alkyl, C₁₋₆ alkoxy, C₂₋₆ alkenyl, C₂₋₆ alkynyl, 4 to 6-membered heterocycloalkyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl;

R⁵ and R⁶ are each independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R⁵ and R⁶ are each optionally substituted with 1, 2, 3, 4 or 5 independently selected R^{b} substituents;

or two adjacent R⁵ substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R⁵ substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

or two adjacent R⁶ substituents on ring A, taken together with the atoms to which they are attached, form a fused phenyl ring, a fused 5-, 6- or 7-membered heterocycloalkyl ring, a fused 5- or 6-membered heteroaryl ring or a fused C₃₋₆ cycloalkyl ring, wherein the fused 5-, 6- or 7-membered heterocycloalkyl ring and fused 5- or 6-membered heteroaryl ring each have 1-4 heteroatoms as ring members selected from N, O and S and wherein the fused phenyl ring, fused 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;

or two R⁶ substituents on the same ring carbon atom of the ring A, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;

each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2 or 3 independently selected R^{d} substituents;

each R^{b} substituent is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1, 2, or 3 independently selected R^{d} substituents;

or two R^{b} substituents attached to the same ring carbon atom taken together with the ring carbon atom to which they are attached form spiro C₃₋₆ cycloalkyl or spiro 4- to 7-membered heterocycloalkyl, each of which is optionally substituted with 1, 2, or 3 independently selected R^{f} substituents;

each R^{c} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2 or 3 R^{f} substituents;

each R^{f} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2 or 3 Rⁿ substituents;

each Rⁿ is independently selected from C₁₋₆ alkyl, C₁₋₆ haloalkyl, halo, CN, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o}, wherein the C₁₋₆ alkyl, C₁₋₆ haloalkyl, phenyl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of Rⁿ is optionally substituted with 1, 2 or 3 R^{q} substituents;

each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1, 2, or 3 independently selected R^{f} substituents;

each R^{e} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{e} are each optionally substituted with 1, 2 or 3 independently selected R^{f} substituents;

each R^{g} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1, 2, or 3 R^{p} substituents;

each R^{p} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r} and S(O)₂NR^{r}R^{r}, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} is optionally substituted with 1, 2 or 3 R^{q} substituents;

or any two R^{a} substituents together with the nitrogen atom to which they are attached form a 4-, 5-, 6-, 7-, 8-, 9- or 10-membered heterocycloalkyl group optionally substituted with 1, 2 or 3 R^{h} substituents;

each R^{h} is independently selected from C₁₋₆ alkyl, C₁₋₆ haloalkyl, C₁₋₆ haloalkoxy, C₃₋₁₀ cycloalkyl, 4-7 membered heterocycloalkyl, C₆₋₁₀ aryl, 5-6 membered heteroaryl, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₆₋₁₀ aryl-C₁₋₄ alkyl-, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, NRⁱC(═NOH)NRⁱRⁱ, NRⁱC(═NCN)NRⁱRⁱ, S(O)Rⁱ, S(O)NRⁱRⁱ, S(O)₂Rⁱ, NRⁱS(O)₂Rⁱ, NRⁱS(O)₂NRⁱRⁱ, and S(O)₂NRⁱRⁱ, wherein the C₁₋₆ alkyl, C₁₋₆ haloalkyl, C₁₋₆ haloalkoxy, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, and (4-7 membered heterocycloalkyl)-C₁₋₄ alkyl- of R^{h} are each optionally substituted by 1, 2, or 3 R^{j} substituents;

each R^{j} is independently selected from C₁₋₄ alkyl, C₃₋₆ cycloalkyl, C₆₋₁₀ aryl, 5- or 6-membered heteroaryl, 4-6 membered heterocycloalkyl, C₂₋₄ alkenyl, C₂₋₄ alkynyl, halo, C₁₋₄ haloalkyl, C₁₋₄ haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR^{k})NR^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}, wherein the C₁₋₄ alkyl, C₃₋₆ cycloalkyl, C₆₋₁₀ aryl, 5- or 6-membered heteroaryl, 4-6 membered heterocycloalkyl, C₂₋₄ alkenyl, C₁₋₄ haloalkyl, and C₁₋₄ haloalkoxy of R^{j} are each optionally substituted with 1, 2 or 3 independently selected R^{q} substituents;

or two R^{h} groups attached to the same carbon atom of the 4- to 10-membered heterocycloalkyl, taken together with the carbon atom to which they are attached, form a C₃₋₆ cycloalkyl or 4- to 6-membered heterocycloalkyl having 1-2 heteroatoms as ring members selected from O, N or S;

or any two R^{c} substituents together with the nitrogen atom to which they are attached form a 4-, 5-, 6-, or 7-membered heterocycloalkyl group optionally substituted with 1, 2, or 3 independently selected R^{h} substituents;

or any two R^{e} substituents together with the nitrogen atom to which they are attached form a 4-, 5-, 6-, or 7-membered heterocycloalkyl group optionally substituted with 1, 2, or 3 independently selected R^{h} substituents;

or any two R^{g} substituents together with the nitrogen atom to which they are attached form a 4-, 5-, 6-, or 7-membered heterocycloalkyl group optionally substituted with 1, 2, or 3 independently selected R^{h} substituents;

or any two Rⁱ substituents together with the nitrogen atom to which they are attached form a 4-, 5-, 6-, or 7-membered heterocycloalkyl group optionally substituted with 1, 2, or 3 independently selected R^{h} substituents;

or any two R^{k} substituents together with the nitrogen atom to which they are attached form a 4-, 5-, 6-, or 7-membered heterocycloalkyl group optionally substituted with 1, 2, or 3 independently selected R^{h} substituents;

or any two R^{o} substituents together with the nitrogen atom to which they are attached form a 4-, 5-, 6-, or 7-membered heterocycloalkyl group optionally substituted with 1, 2, or 3 independently selected R^{h} substituents; and

or any two R^{r} substituents together with the nitrogen atom to which they are attached form a 4-, 5-, 6-, or 7-membered heterocycloalkyl group optionally substituted with 1, 2, or 3 independently selected R^{h} substituents;

each Rⁱ, R^{k}, R^{o} or R^{r} is independently selected from H, C₁₋₆ alkyl, C₁₋₆ haloalkyl, C₃₋₆ cycloalkyl, C₆₋₁₀ aryl, 4-6 membered heterocycloalkyl, 5 or 6-membered heteroaryl, C₁₋₄ haloalkyl, C₂₋₄ alkenyl, and C₂₋₄ alkynyl, wherein the C₁₋₄ alkyl, C₁₋₆ haloalkyl, C₃₋₆ cycloalkyl, C₆₋₁₀ aryl, 4-6 membered heterocycloalkyl, 5 or 6-membered heteroaryl, C₂₋₄ alkenyl, and C₂₋₄ alkynyl of Rⁱ, R^{k}, R^{o} or R^{r} are each optionally substituted with 1, 2 or 3 R^{q} substituents;

each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NHR¹² and NR¹²R¹², wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl and 4-6 membered heterocycloalkyl and each R¹² is independently C₁₋₆ alkyl;

the subscript n is an integer of 0, 1, 2, 3, 4, 5, 6, 7 or 8;

the subscript m is an integer of 0, 1, 2, 3, 4, 5, 6, 7 or 8;

each subscript p is independently an integer of 1, 2, 3 or 4;

each subscript t is independently an integer of 0, 1, 2, 3 or 4;

with the provisos:

(i) when L¹ is a bond and

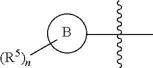

is phenyl or 2,3-dihydro-1,4-benzodioxin-6-yl, then

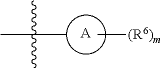

is not

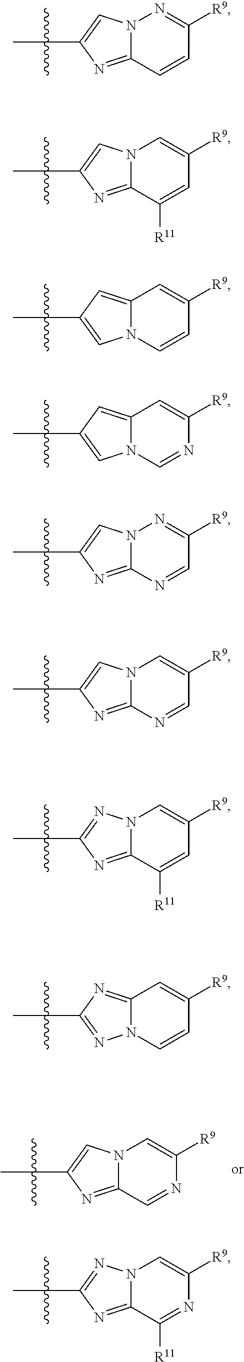

wherein each R⁹ is independently (2-hydroxyethylamino)methyl or (2-carboxy-1-piperidinyl)methyl and each R¹¹ is independently H, halo, CN, C₁₋₆ alkyl, C₁₋₆ alkoxy, —NHC₁₋₆ alkyl or benzyloxy, wherein the C₁₋₆ alkyl, C₁₋₆ alkoxy, —NHC₁₋₆ alkyl and benzyloxy of R¹¹ are each optionally substituted with halo, CN, C₁₋₆ alkyl or C₁₋₆ alkoxy;

(ii) when L¹ is a bond and

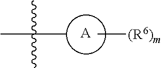

is phenyl or 2,3-dihydro-1,4-benzodioxin-6-yl,

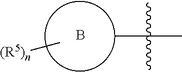

is not any of the moieties set forth in proviso (i) above for

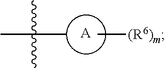

(iii) when L¹ is a bond, then

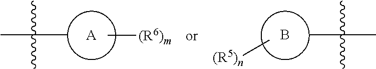

is not

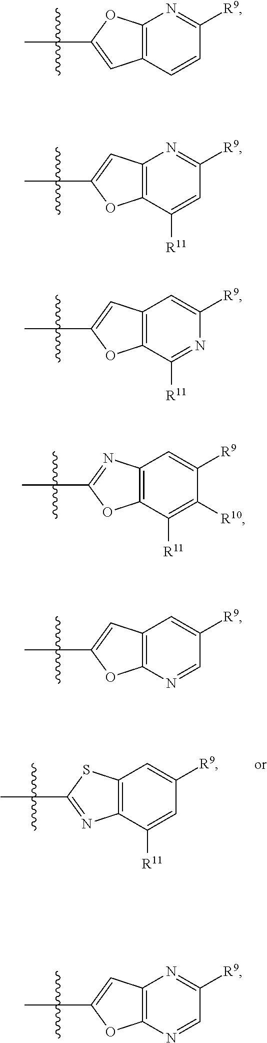

wherein each R⁹ is independently (2-hydroxyethylamino)methyl or (2-carboxy-1-piperidinyl)methyl; each R¹¹ is independently H or C₁₋₆ alkyl and R¹⁰ is H, C₁₋₆ alkoxy, benzyloxy, morpholinoethoxy or 2-pyridylmethyloxy, wherein the C₁₋₆ alkoxy, benzyloxy and 2-pyridylmethyloxy of R¹⁰ are each optionally substituted with CN;

(iv) when L¹ is a bond, then or

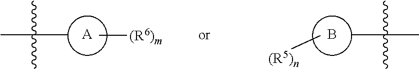

is not

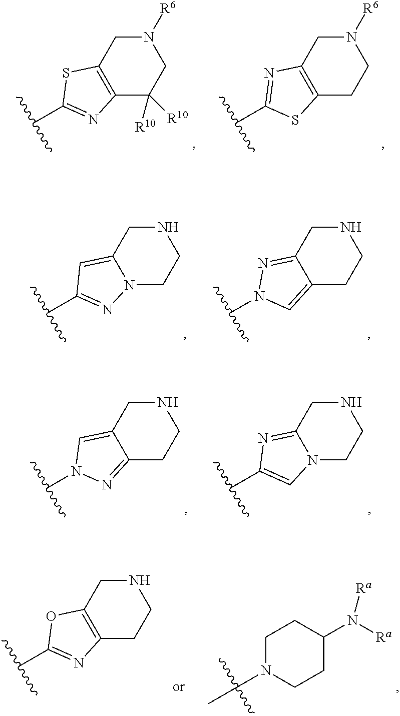

wherein R¹⁰ is H or C₁₋₆ alkyl;

(v) when L¹ is —NHC(O)—, then

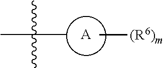

is not

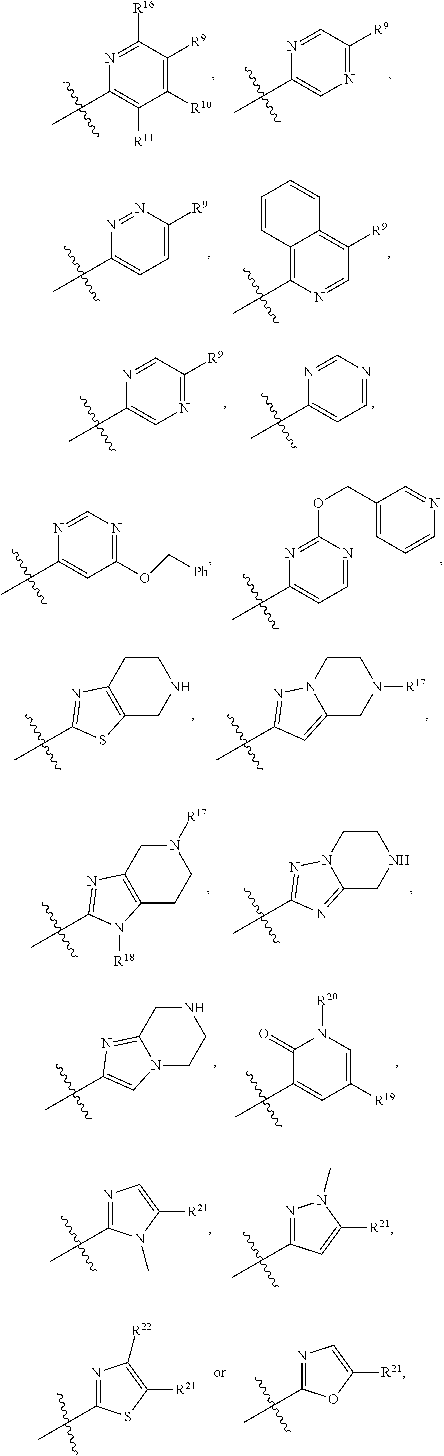

wherein each R⁹ is independently H, methyl, (2-hydroxyethylamino)methyl or (2-carboxy-1-piperidinyl)methyl; R¹⁰ is H, methyl, CN, methoxy, cyclopropylmethoxy, benzyloxy, (2-cyanophenyl)methoxy, 2-pyridylmethoxy, 3-pyridylmethoxy, (2-hydroxyethylamino)methyl or (2-carboxy-1-piperidinyl)methyl; R¹¹ is H, halo, methyl or dimethylamino; R¹⁶ is H or methyl; each R¹⁷ is independently H, 2-hydroxyethyl or carboxymethyl; R¹⁸ is H or methyl; R¹⁹ is (2-hydroxyethylamino)methyl or (2-carboxy-1-piperidinyl)methyl; R²⁰ is C₁₋₆ alkyl; each R²¹ is independently 2-hydroxyethylamino)methyl or (2-carboxy-1-piperidinyl)methyl; and R²² is H or Cl;

(vi) when L¹ is —NH— and

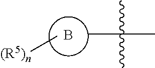

is phenyl, 2,3-dihydro-1,4-benzodioxin-6-yl, cyclohexyl or 1-cyclohexenyl, then

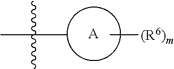

is not

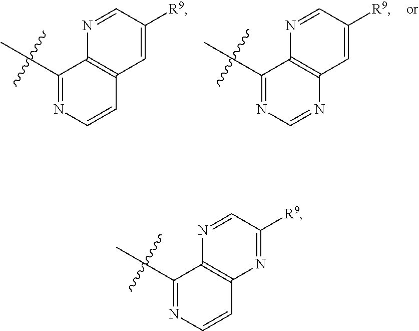

wherein each R⁹ is independently (2-hydroxyethylamino)methyl or (2-carboxy-1-piperidinyl)methyl;

(vii) when L¹ is —CH₂O—, ring B is phenyl or thienyl, and the subscript n is 1 or 2, then R⁵ is not a substituent independently selected from H, —OCH₃, —OH, —OCH₂CH₃, —O(CH₂)OCH₃, —OCH₂CH═CH₂, —O(CH₂)₂CH₃, —O(CH₂)₂morpholinyl or F; and

(viii) when L¹ is —CH₂O—, ring B is phenyl or thienyl, and the subscript n is 2, then two R⁵ substituents attached to adjacent ring carbon atoms of ring B do not form —OCH₂O— or —OCH₂CH₂O—; and

wherein the compound, or a pharmaceutically acceptable salt or a stereoisomer thereof inhibits PD-1/PD-L1 interaction.

In some embodiments, any two Rⁱ substituents together with the nitrogen atom to which they are attached form a 4-, 5-, 6-, 7-, 8-, 9- or 10-membered heterocycloalkyl group optionally substituted with 1, 2, or 3 independently selected R^{q} substituents;

or any two R^{k} substituents together with the nitrogen atom to which they are attached form a 4-, 5-, 6-, 7-, 8-, 9- or 10-membered heterocycloalkyl group optionally substituted with 1, 2, or 3 independently selected R^{q} substituents.

In some embodiments, provided herein is a compound or a pharmaceutically acceptable salt or a stereoisomer thereof, having an IC₅₀ of less than 1 μM in a PD-L1 binding assay. In some embodiments, the compounds as disclosed herein have an IC₅₀ of less than 0.9, 0.8, 0.7, 0.6, 0.5, 0.4, 0.3, 0.2, 0.1, 0.01, 0.005, 0.004, 0.003, 0.002, or 0.001 μM. For example, the PD-L1 binding assay can be a PD-1/PD-L1 Homogeneous Time-Resolved Fluorescence (HTRF) binding assay as described in Example A. In some embodiments, the subscript m is an integer of 0, 1, 2, 3, 4, 5, 6, 7 or 8 and the subscript n is an integer of 1, 2, 3, 4, 5, 6, 7 or 8; or the subscript m is an integer of 1, 2, 3, 4, 5, 6, 7 or 8 and the subscript n is an integer of 0, 1, 2, 3, 4, 5, 6, 7 or 8; or the subscripts m and n are each independently an integer of 1, 2, 3, 4, 5, 6, 7 or 8.

In some embodiments,

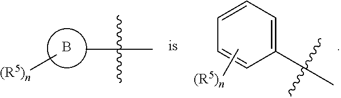

In some embodiments, provided herein is a compound having Formula (II):

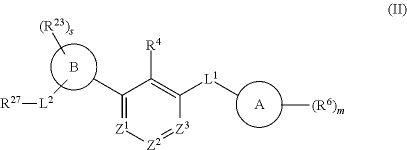

or a pharmaceutically acceptable salt or a stereoisomer thereof, wherein:

L² is a bond, —(CR²⁹R³⁰)C(O)NR²⁸(CR²⁹R³⁰)ₜ—, —(CR²⁹R³⁰)ₜNR²⁸C(O)(CR²⁹R³⁰)ₜ—, O, —(CR²⁹R³⁰)_{q}—, —(CR²⁹R³⁰)_{q}—O—, —O(CR²⁹R³⁰)_{q}—, —(CR²⁹R³⁰)_{q}—O—(CR²⁹R³⁰)_{q}—, —NR²⁸—, —(CR²⁹R³⁰)ₜNR²⁸(CR²⁹R³⁰)ₜ—, —NH—, —(CR²⁹R³⁰)ₜNH(CR²⁹R³⁰)ₜ—, —CH═CH—, —C≡C—, —SO₂—, —(CR²⁹R³⁰)ₜSO₂(CR²⁹R³⁰)ₜ—, —(CR²⁹R³⁰)ₜSO₂NR²⁸(CR²⁹R³⁰)ₜ—, —(CR²⁹R³⁰)ₜNR²⁸SO₂(CR²⁹R³⁰)ₜ—, —(CR²⁹R³⁰)ₜNR²⁸C(O)O(CR²⁹R³⁰)ₜ—, —NR²⁸C(O)O—, —(CR²⁹R³⁰)ₜO(CO)NR²⁸(CR²⁹R³⁰)ₜ—, —O(CO)NR²⁸—, —NR²⁸C(O)NR²⁸— or —(CR²⁹R³⁰)ₜNR²⁸C(O)NR²⁸(CR²⁹R³⁰)ₜ;

each R²³ is independently C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl or —N(C₁₋₄ alkyl)₂;

R²⁷ is C₆₋₁₀ aryl, C₃₋₁₀ cycloalkyl, 5-14 membered heteroaryl, 4-11 membered heterocycloalkyl, each of which is optionally substituted with 1, 2, 3, 4 or 5 independently selected R^{b} substituents;

each R²⁸ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;

R²⁹ and R³⁰ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R²⁹ or R³⁰ are each optionally substituted with 1, 2 or 3 independently selected R^{q} substituents;

or R²⁹ and R³⁰ taken together with the carbon atom to which they are attached form spiro C₃₋₆ cycloalkyl or spiro 4- to 7-membered heterocycloalkyl, each of which is optionally substituted with 1, 2, or 3 independently selected R^{q} substituents;

the subscript s is an integer of 0, 1, 2, 3 or 4; and

each subscript q is independently an integer of 1, 2, 3 or 4.

In some embodiments, provided herein is a compound having Formula (IIa):

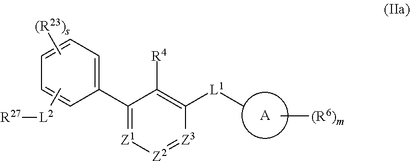

or a pharmaceutically acceptable salt or a stereoisomer thereof. In some embodiments, Z¹ is CR¹, Z² is CR² and Z³ is CR³.

In some embodiments, provided herein is a compound having Formula (III):

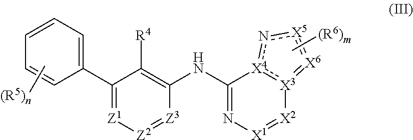

or a pharmaceutically acceptable salt or a stereoisomer thereof, wherein:

X¹, X², X³, X⁴ and X⁶ are each independently C or N, with the proviso that no more than two of X¹, X², X³ and X⁴ are simultaneously N;

X⁵ is C, N, O or S;

[symbol] is a single or a double bond to maintain the fused 5- and 6-membered rings being aromatic.

In some embodiments, provided herein is a compound having Formula (IIIa):

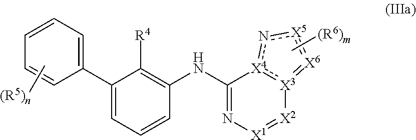

or a pharmaceutically acceptable salt or a stereoisomer thereof.

In some embodiments, provided herein is a compound having Formula (IV):

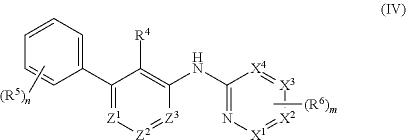

or a pharmaceutically acceptable salt or a stereoisomer thereof, wherein:

X¹, X², X³ and X⁴ are each independently C or N, with the proviso that no more than two of X¹, X², X³ and X⁴ are simultaneously N.

In some embodiments, provided herein is a compound having Formula (IVa):

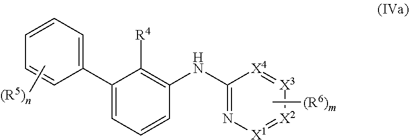

or a pharmaceutically acceptable salt or a stereoisomer thereof.

In some embodiments, provided herein is a compound having Formula (V):

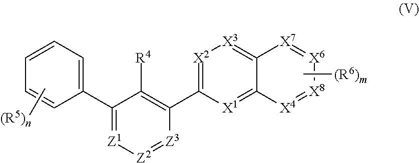

or a pharmaceutically acceptable salt or a stereoisomer thereof, wherein:

X¹, X², X³, X⁴, X⁶, X⁷ and X⁸ are each independently C or N, with the proviso that no more than three of X⁴, X⁶, X⁷ and X⁸ are simultaneously N.

In some embodiments, provided herein is a compound having Formula (Va):

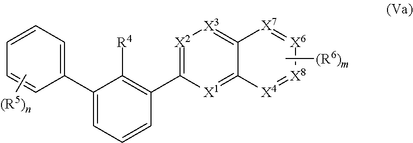

or a pharmaceutically acceptable salt or a stereoisomer thereof.

In some embodiments, provided herein is a compound having Formula (VI):

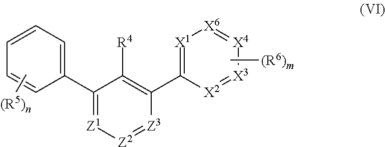

or a pharmaceutically acceptable salt or a stereoisomer thereof, wherein:

X¹, X², X³, X⁴ and X⁶ are each independently C or N, with the proviso that no more than three of X¹, X², X³, X⁴ and X⁶ are simultaneously N.

In some embodiments, provided herein is a compound having Formula (VIa):

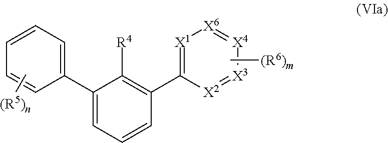

or a pharmaceutically acceptable salt or a stereoisomer thereof.

In some embodiments, provided herein is a compound having Formula (VII):

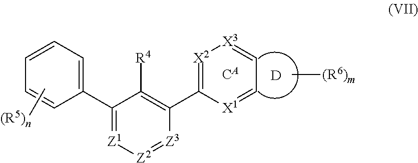

or a pharmaceutically acceptable salt or a stereoisomer thereof, wherein:

X¹, X² and X³ are each independently C or N; and

ring C^{A} is aromatic and ring D is fused 5- or 6-membered heterocycloalkyl.

In some embodiments, provided herein is a compound having Formula (VIIa):

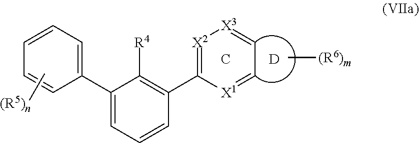

or a pharmaceutically acceptable salt or a stereoisomer thereof.

In some embodiments, provided herein is a compound having Formula (VIII):

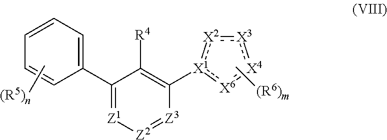

or a pharmaceutically acceptable salt or a stereoisomer thereof, wherein:

X¹ is N or C;

X², X³, X⁴ and X⁶ are each independently C, N, O or to maintain the 5-membered ring A being aromatic.

In some embodiments, provided herein is a compound having Formula (VIIIa):

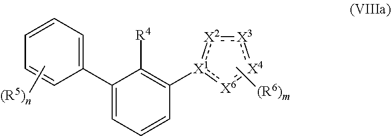

or a pharmaceutically acceptable salt or a stereoisomer thereof.

In some embodiments, provided herein is a compound having Formula (IX):

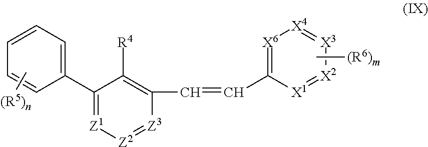

or a pharmaceutically acceptable salt or a stereoisomer thereof, wherein:

X¹, X² and X³ are each independently C or N;

X⁴ is CR²⁴ or N;

X⁶ is CR²⁵ or N;

or R²⁴ and R²⁵ together with the carbon atoms to which they are attached form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 R^{b} substituents.

In some embodiments, provided herein is a compound having Formula (IXa):

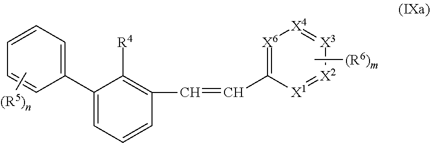

or a pharmaceutically acceptable salt or a stereoisomer thereof.

In some embodiments, provided herein is a compound having Formula (X):

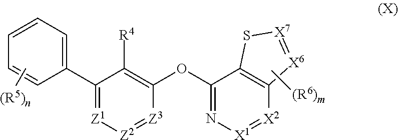

or a pharmaceutically acceptable salt or a stereoisomer thereof, wherein:

X¹, X², X⁶ and X⁷ are each independently C or N.

In some embodiments, Z¹ is CR¹, Z² is CR² and Z³ is CR³.

In some embodiments,

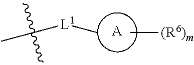

is selected from:

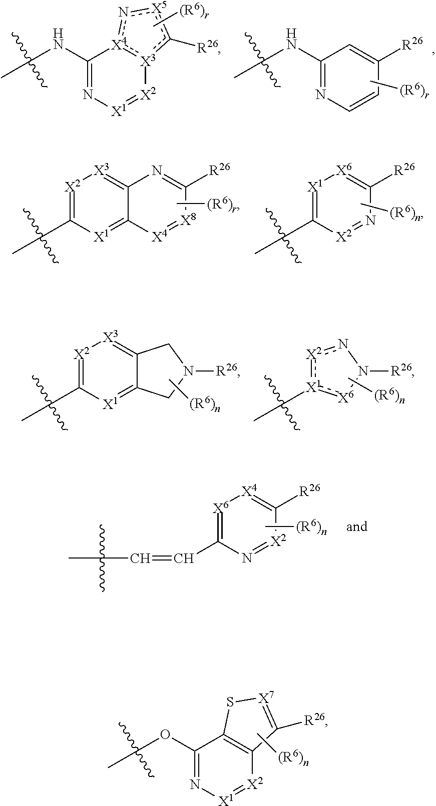

wherein

X¹, X², X³, X⁴, X⁶, X⁷ and X⁸ are each independently C or N;

X⁵ is C, N, O or S;

each R²⁶ is independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R²⁶ is optionally substituted with 1, 2, 3, 4 or 5 independently selected R^{b} substituents; and

each subscript r is independently an integer of 1, 2, 3, 4, 5, 6 or 7.

In some embodiments,

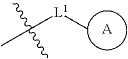

is selected from:

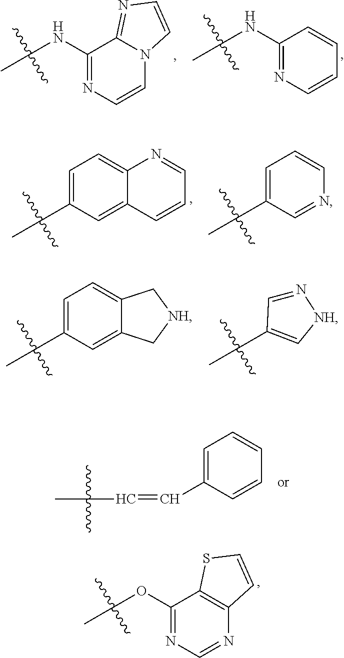

each of which is optionally substituted with 1 to 5 independently selected R⁶ substituents.

In some embodiments,

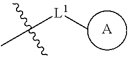

is selected from:

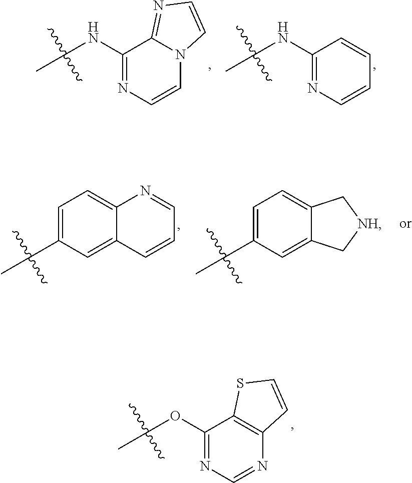 [SMILES: *Nc1nccn2ccnc12 ; *Nc1ccccn1 ; *c1ccc2ncccc2c1 ; *c1ccc2c(c1)CNC2 ; "or" ; *Oc1ncnc2ccsc12]

each of which is optionally substituted with 1 to 5 independently selected R⁶ substituents.

In some embodiments,

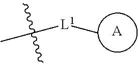

is selected from:

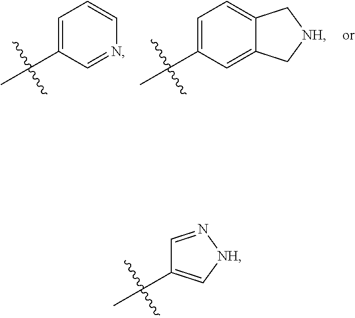 [SMILES: *c1cccnc1 ; *c1ccc2c(c1)CNC2 ; *c1cn[nH]c1]

each of which is optionally substituted with 1 to 5 independently selected R⁶ substituents.

In some embodiments,

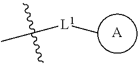

is selected from:

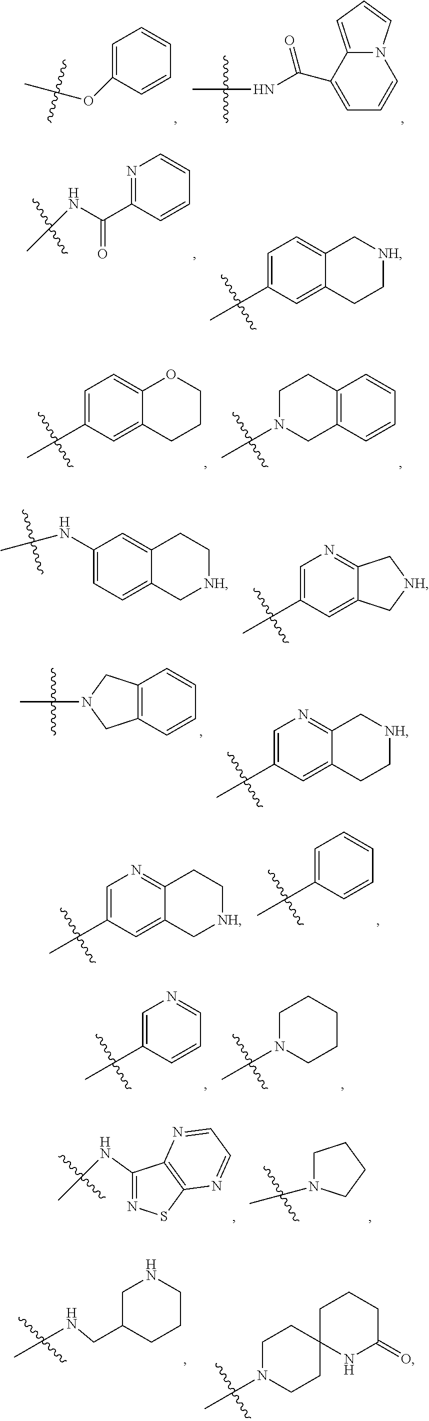

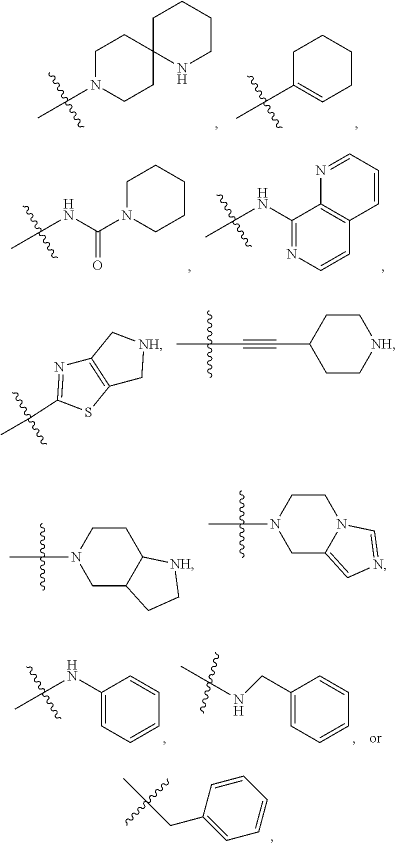

each of which is optionally substituted with 1, 2, 3, 4 or 5 independently selected R⁶ substituents.

In some embodiments, ring B:

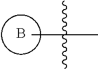

is selected from:

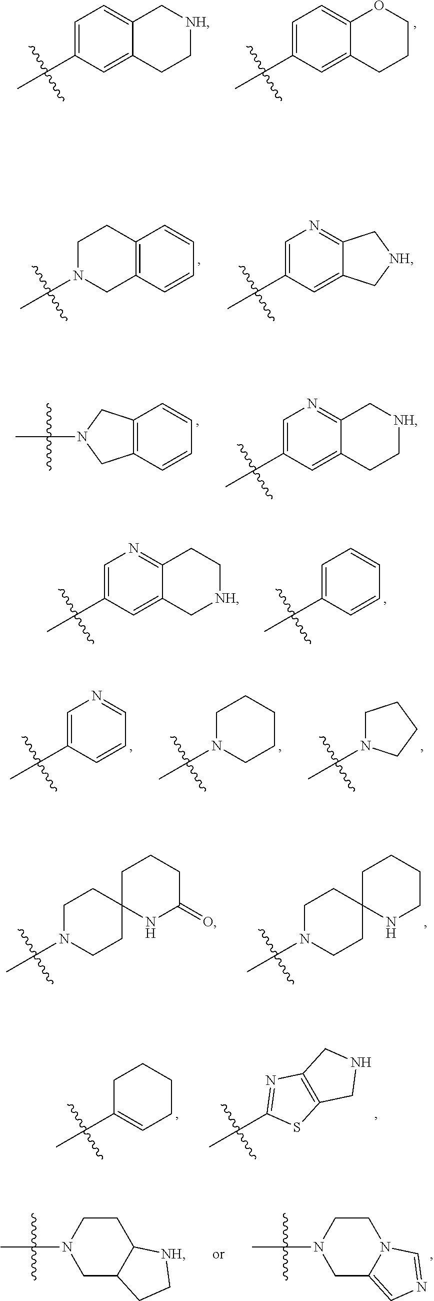

each of which is optionally substituted with 1 to 5 independently selected R⁵ substituents.

In some embodiments,

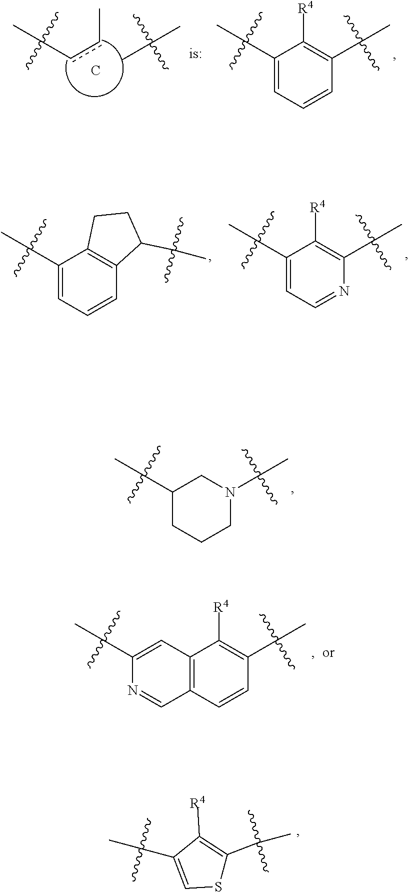

each of which is optionally substituted with 1, 2 or 3 independently selected R³¹ substituents.

In some embodiments, L¹ is a bond, —O—, —NHC(O)—, —NH—, —CH₂NH—, or —CH₂—.

In some embodiments, L² is a bond.

In some embodiments, L³ is a bond, —O—, —NHC(O)—, —NH—, —CH₂NH—, or —CH₂—.

In some embodiments, provided herein is a compound of Formula (XI):

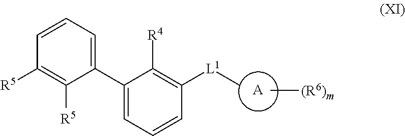

or a pharmaceutically acceptable salt or a stereoisomer thereof, wherein:

L¹ is a bond, O, —NR¹³—, or —CH═CH—;

ring A is

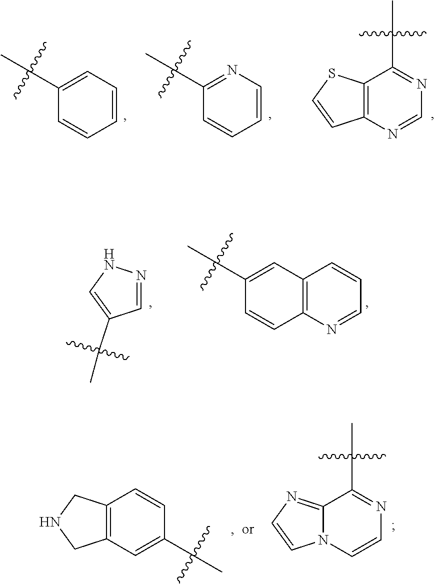

wherein [symbol] indicates the point of attachment of ring A to L¹;

each R¹³ is independently H, C₁₋₆ haloalkyl or C₁₋₆ alkyl;

R⁴ is halo or C₁₋₆ alkyl;

each R⁵ is independently selected from halo and OR^{a};

each R⁶ is independently selected from halo, C₁₋₆ alkyl, C₂₋₆ alkenyl, C₂₋₆ alkynyl, C₁₋₆ haloalkyl, C₁₋₆ haloalkoxy, 5-14 membered heteroaryl, 4-10 membered heterocycloalkyl, (5-14 membered heteroaryl)-C₁₋₄ alkyl-, (4-10 membered heterocycloalkyl)-C₁₋₄ alkyl-, CN, NO₂, OR^{a}, C(O)R^{a}, C(O)NR^{a}R^{a}, C(O)OR^{a}, NHR^{a}, NR^{a}R^{a}, NR^{a}C(O)R^{a}, and NR^{a}C(O)OR^{a}, wherein the C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, and (4-10 membered heterocycloalkyl)-C₁₋₄ alkyl- of R⁶ are each optionally substituted with 1, 2 or 3 independently selected R^{b} substituents;

each R^{a} is independently selected from H, C₁₋₆ alkyl, C₁₋₆ haloalkyl, C₂₋₆ alkenyl, and C₂₋₆ alkynyl;

each R^{b} substituent is independently selected from halo, C₁₋₆ alkyl, C₁₋₆ haloalkyl, C₁₋₆ haloalkoxy, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, CN, OH, NH₂, NO₂, OR^{c}, C(O)R^{c}, C(O)NR^{c}R^{c}, C(O)OR^{c}, NHR^{c}, NR^{c}R^{c}, NR^{c}C(O)R^{c}, and NR^{c}C(O)OR^{c};

each R^{c} is independently selected from H, C₁₋₆ alkyl, C₁₋₆ haloalkyl, C₂₋₆ alkenyl, and C₂₋₆ alkynyl, wherein the C₁₋₆ alkyl, C₁₋₆ haloalkyl, C₂₋₆ alkenyl, and C₂₋₆ alkynyl R^{c} are each optionally substituted with 1, 2 or 3 R^{f} substituents;

each R^{f} is independently selected from C₁₋₆ alkyl, C₁₋₆ haloalkyl, C₂₋₆ alkenyl, C₂₋₆ alkynyl, halo, CN, OR^{g}, C(O)R^{g}, C(O)NR^{g}R^{g}, C(O)OR^{g}, NHR^{g}, NR^{g}R^{g}, NR^{g}C(O)R^{g}, and NR^{g}C(O)OR^{g};

each R^{g} is independently selected from H, C₁₋₆ alkyl, C₁₋₆ haloalkyl, C₂₋₆ alkenyl, and C₂₋₆ alkynyl;

or any two R^{c} substituents together with the nitrogen atom to which they are attached form a 4-, 5-, 6-, or 7-membered heterocycloalkyl group optionally substituted with 1, 2, or 3 independently selected R^{h} substituents;

each R^{h} is independently selected from C₁₋₆ alkyl, C₁₋₆ haloalkyl, C₁₋₆ haloalkoxy, C₂₋₆ alkenyl, C₂₋₆ alkynyl, halo, CN, ORⁱ, C(O)Rⁱ, C(O)NRⁱRⁱ, C(O)ORⁱ, NHRⁱ, NRⁱRⁱ, NRⁱC(O)Rⁱ, and NRⁱC(O)ORⁱ;

each Rⁱ is independently selected from H, C₁₋₆ alkyl, and C₁₋₆ haloalkyl; and the subscript m is an integer of 0, 1, 2, or 3.

In some embodiments, provided herein is a compound of Formula (XI), or a pharmaceutically acceptable salt or a stereoisomer thereof, wherein:

L¹ is a bond, O, —NR¹³—, or —CH═CH—;

ring A is

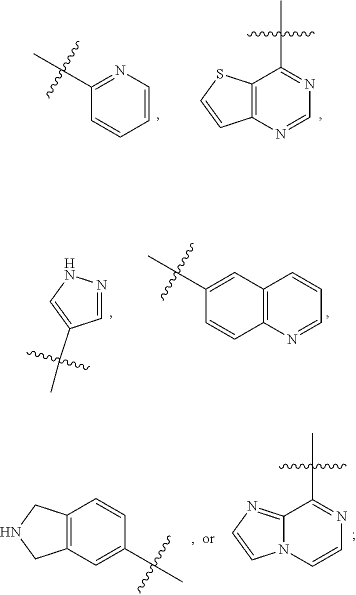 [SMILES: *c1ccccn1 ; *c1ncnc2ccsc12 ; *c1cn[nH]c1 ; *c1ccc2ncccc2c1 ; *c1ccc2c(c1)CNC2 ; *c1nccn2ccnc12]

wherein [symbol] indicates the point of attachment of ring A to L¹;

each R¹³ is independently H, C₁₋₆ haloalkyl or C₁₋₆ alkyl;

R⁴ is halo or C₁₋₆ alkyl;

each R⁵ is independently selected from halo and OR^{a};

each R⁶ is independently selected from halo, C₁₋₆ alkyl, C₂₋₆ alkenyl, C₂₋₆ alkynyl, C₁₋₆ haloalkyl, C₁₋₆ haloalkoxy, 5-14 membered heteroaryl, 4-10 membered heterocycloalkyl, (5-14 membered heteroaryl)-C₁₋₄ alkyl-, (4-10 membered heterocycloalkyl)-C₁₋₄ alkyl-, CN, NO₂, OR^{a}, C(O)R^{a}, C(O)NR^{a}R^{a}, C(O)OR^{a}, NHR^{a}, NR^{a}R^{a}, NR^{a}C(O)R^{a}, and NR^{a}C(O)OR^{a}, wherein the C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, and (4-10 membered heterocycloalkyl)-C₁₋₄ alkyl- of R⁶ are each optionally substituted with 1, 2 or 3 independently selected R^{b} substituents;

each R^{a} is independently selected from H, C₁₋₆ alkyl, C₁₋₆ haloalkyl, C₂₋₆ alkenyl, and C₂₋₆ alkynyl;

each R^{b} substituent is independently selected from halo, C₁₋₆ alkyl, C₁₋₆ haloalkyl, C₁₋₆ haloalkoxy, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, CN, OH, NH₂, NO₂, OR^{c}, C(O)R^{c}, C(O)NR^{c}R^{c}, C(O)OR^{c}, NHR^{c}, NR^{c}R^{c}, NR^{c}C(O)R^{c}, and NR^{c}C(O)OR^{c};

each R^{c} is independently selected from H, C₁₋₆ alkyl, C₁₋₆ haloalkyl, C₂₋₆ alkenyl, and C₂₋₆ alkynyl, wherein the C₁₋₆ alkyl, C₁₋₆ haloalkyl, C₂₋₆ alkenyl, and C₂₋₆ alkynyl R^{c} are each optionally substituted with 1, 2 or 3 R^{f} substituents;

each R^{f} is independently selected from C₁₋₆ alkyl, C₁₋₆ haloalkyl, C₂₋₆ alkenyl, C₂₋₆ alkynyl, halo, CN, OR^{g}, C(O)R^{g}, C(O)NR^{g}R^{g}, C(O)OR^{g}, NHR^{g}, NR^{g}R^{g}, NR^{g}C(O)R^{g}, and NR^{g}C(O)OR^{g};

each R^{g} is independently selected from H, C₁₋₆ alkyl, C₁₋₆ haloalkyl, C₂₋₆ alkenyl, and C₂₋₆ alkynyl;

or any two R^{c} substituents together with the nitrogen atom to which they are attached form a 4-, 5-, 6-, or 7-membered heterocycloalkyl group optionally substituted with 1, 2, or 3 independently selected R^{h} substituents;

each R^{h} is independently selected from C₁₋₆ alkyl, C₁₋₆ haloalkyl, C₁₋₆ haloalkoxy, C₂₋₆ alkenyl, C₂₋₆ alkynyl, halo, CN, ORⁱ, C(O)Rⁱ, C(O)NRⁱRⁱ, C(O)ORⁱ, NHRⁱ, NRⁱRⁱ, NRⁱC(O)Rⁱ, and NRⁱC(O)ORⁱ;

each Rⁱ is independently selected from H, C₁₋₆ alkyl, and C₁₋₆ haloalkyl; and the subscript m is an integer of 0, 1, 2, or 3.

In some embodiments, provided herein is a compound of Formula (XI), or a pharmaceutically acceptable salt or a stereoisomer thereof, wherein:

L¹ is a bond, O, —NH—, or —CH═CH—;

ring A is

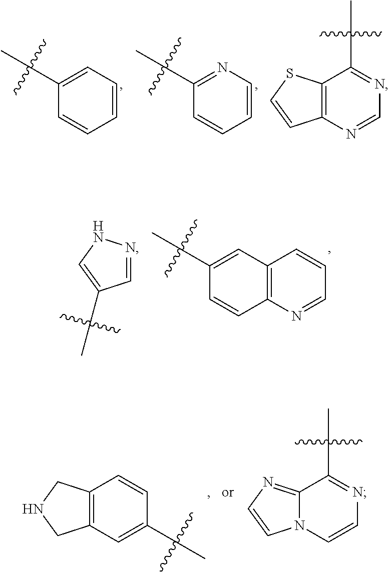

wherein [symbol] indicates the point of attachment of ring A to L¹;

R⁴ is halo or C₁₋₆ alkyl;

each R⁵ is independently selected from halo and OR^{a};

each R⁶ is independently selected from halo, C₁₋₆ alkyl, (4-10 membered heterocycloalkyl)-C₁₋₄ alkyl-, and OR^{a}, wherein the C₁₋₆ alkyl and (4-10 membered heterocycloalkyl)-C₁₋₄ alkyl- of R⁶ are each optionally substituted with 1, 2 or 3 independently selected R^{b} substituents;

each R^{a} is independently selected from H and C₁₋₆ alkyl;

each R^{b} substituent is independently selected from halo, C₁₋₆ alkyl, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, C(O)OR^{c}, NHR^{c}, and NR^{c}R^{c};

each R^{c} is independently selected from H and C₁₋₆ alkyl, wherein the C₁₋₆ alkyl is optionally substituted with 1 or 2 R^{f} substituents;

each R^{f} is independently selected from C₁₋₆ alkyl and OR^{g};

each R^{g} is independently selected from H and C₁₋₆ alkyl;

or any two R^{c} substituents together with the nitrogen atom to which they are attached form a 4-, 5-, 6-, or 7-membered heterocycloalkyl group optionally substituted with 1, 2, or 3 independently selected R^{h} substituents;

each R^{h} is C(O)ORⁱ;

each Rⁱ is independently selected from H and C₁₋₆ alkyl; and the subscript m is an integer of 0, 1, 2, or 3.

In some embodiments, provided herein is a compound of Formula (XI), or a pharmaceutically acceptable salt or a stereoisomer thereof, wherein:

L¹ is a bond, 0, —NH—, or —CH═CH—;

ring A is

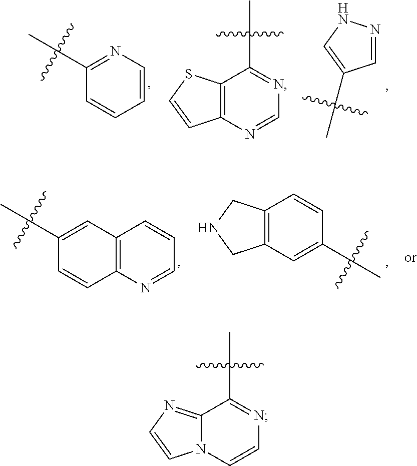

wherein [symbol] indicates the point of attachment of ring A to L¹;

R⁴ is halo or C₁₋₆ alkyl;

each R⁵ is independently selected from halo and OR^{a};

each R⁶ is independently selected from halo, C₁₋₆ alkyl, (4-10 membered heterocycloalkyl)-C₁₋₄ alkyl-, and OR^{a}, wherein the C₁₋₆ alkyl and (4-10 membered heterocycloalkyl)-C₁₋₄ alkyl- of R⁶ are each optionally substituted with 1, 2 or 3 independently selected R^{b} substituents;

each R^{a} is independently selected from H and C₁₋₆ alkyl;

each R^{b} substituent is independently selected from halo, C₁₋₆ alkyl, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, C(O)OR^{c}, NHR^{c}, and NR^{c}R^{c};

each R^{c} is independently selected from H and C₁₋₆ alkyl, wherein the C₁₋₆ alkyl is optionally substituted with 1 or 2 R^{f} substituents;

each R^{f} is independently selected from C₁₋₆ alkyl and OR^{g};

each R^{g} is independently selected from H and C₁₋₆ alkyl;

or any two R^{c} substituents together with the nitrogen atom to which they are attached form a 4-, 5-, 6-, or 7-membered heterocycloalkyl group optionally substituted with 1, 2, or 3 independently selected R^{h} substituents;

each R^{h} is C(O)ORⁱ;

each Rⁱ is independently selected from H and C₁₋₆ alkyl; and

the subscript m is an integer of 0, 1, 2, or 3.

In some embodiments, L is a bond. In some embodiments, L is O. In some embodiments, L is NH. In some embodiments, L is —CH═CH—.

In some embodiments, R⁴ is C₁₋₆ alkyl. In some embodiments, R⁴ is halo.

In some embodiments, R⁵ is OR^{a}. In some embodiments, R⁵ is halo.

In some embodiments, each R⁶ is independently selected from C₁₋₆ alkyl, and (4-10 membered heterocycloalkyl)-C₁₋₄ alkyl-, wherein the C₁₋₆ alkyl and (4-10 membered heterocycloalkyl)-C₁₋₄ alkyl- of R⁶ are each optionally substituted with 1 or 2 independently selected R^{b} substituents. In some embodiments, each R⁶ is 2-hydroxyethylaminomethyl, pyrrolidin-2-ylmethyl, methylpiperidine-2-carboxylic acid, or aminomethyl.

In some embodiments, ring A is

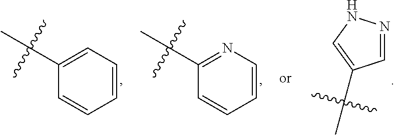 [SMILES: *c1ccccc1 ; *c1ccccn1 ; *c1cn[nH]c1]

In some embodiments, ring A is

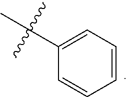 [SMILES: *c1ccccc1]

In some embodiments, ring A is

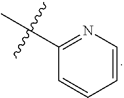 [SMILES: *c1ccccn1]

In some embodiments, ring A is

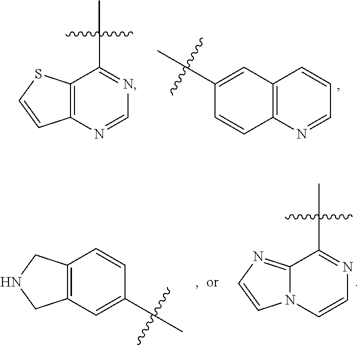 [SMILES: *c1ncnc2ccsc12 ; *c1ccc2ncccc2c1 ; *c1ccc2c(c1)CNC2 ; *c1nccn2ccnc12]

In some embodiments, L¹ is a bond, NH, —NR¹³—, —CH₂O—, —OCH₂—, —NR¹³CH₂—, —CH₂NR¹³—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, —(CR¹⁴R¹⁵)ₜNR¹³(CR¹⁴R¹⁵)ₜ—, —NR¹³(CR¹⁴R¹⁵)ₜ—, or —(CR¹⁴R¹⁵)ₜNR¹³—.

In some embodiments, L² is a bond, NH, —NR²⁸—, —CH₂O—, —OCH₂—, —NR²⁸CH₂—, —CH₂NR²⁸—, —(CR²⁹R³⁰)ₚ—O—, —O(CR²⁹R³⁰)ₚ—, —(CR²⁹R³⁰)ₚ—O—(CR²⁹R³⁰)ₚ—, —(CR²⁹R³⁰)ₜNR²⁸(CR²⁹R³⁰)ₜ—, —NR²⁸(CR²⁹R³⁰)ₜ—, or —(CR²⁸R³⁰)ₜNR²⁸

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ₋includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

The term “alkoxy”, employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “amino” refers to a group of formula —NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CCl₃, CHCl₂, C₂Cl₅ and the like. In some embodiments, the haloalkyl group is a fluoroalkyl group.

The term “haloalkoxy”, employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfido” refers to a sulfur atom as a divalent substituent, forming a thiocarbonyl group (C═S) when attached to carbon.

The term “aromatic” refers to a carbocycle or heterocycle having one or more polyunsaturated rings having aromatic character (i.e., having (4n+2) delocalized 71 (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, indanyl, indenyl and the like. Also included in the definition of aryl are moieties that have one or more cycloalkyl or heterocycloalkyl rings fused (i.e., having a bond in common with) to the aryl ring, for example, cyclopentyl, cyclohexyl, azetidinyl, pyrrolidinyl, piperazinyl, or oxazolidinyl fused with phenyl, naphthyl, and the like. An aryl group containing a fused cycloalkyl or heterocycloalkyl ring can be attached through any ring-forming atom, for example, a ring-forming atom of the fused aromatic ring In some embodiments, aryl groups have from 6 to about 10 ring carbon atoms. In some embodiments aryl groups have 6 carbon atoms. In some embodiments aryl groups have 10 ring carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. Also included in the definition of heteroaryl are moieties that have one or more cycloalkyl or heterocycloalkyl rings fused (i.e., having a bond in common with) to the heteroaryl ring, for example, cyclobutyl, cyclopentyl, cyclohexyl, azetidinyl, pyrrolidinyl, piperazinyl, or oxazolidinyl fused with pyridyl, thiophenyl, and the like. A heteroaryl group containing a fused cycloalkyl or heterocycloalkyl ring can be attached through any ring-forming atom, for example, a ring-forming atom of the fused heteroaromatic ring. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-14, or 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, benzothiophenyl, benzofuranyl, benzisoxazolyl, imidazo[1,2-b]thiazolyl, purinyl, thienopyrimidinyl (e.g., thieno[3,2-d]pyrimidin-7-yl), imidazopyrazinyl (e.g., imidazo[1,2-a]pyrazin-3-yl) and the like.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6, 7, 8, 9, 10, 11, 12, 13, or 14 ring-forming carbons (C₃₋₁₄). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom, for example, a ring-forming atom of the cycloalkyl ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur oxygen and phosphorus, and which has 4-14 ring members, 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic or polycyclic (e.g., having two or three fused or bridged rings) ring systems or spirorcycles. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide, etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, isoindolinyl, and thiomorpholino.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### II. Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds of Formula I can be synthesized using a process shown in Scheme 1. In Scheme 1, a suitable halo (Hal¹)-substituted arene 1-1 can react with a coupling reagent 1-2 (where M is, e.g., —B(OR)₂) to provide the product of formula I under standard metal catalyzed cross-coupling reaction conditions (such as Suzuki coupling reaction, e.g., in the presence of a palladium catalyst (e.g., [1,1′-bis(diphenylphosphino)ferrocene]dichloropalladium(II)) and a base (e.g., a bicarbonate or a carbonate base)).

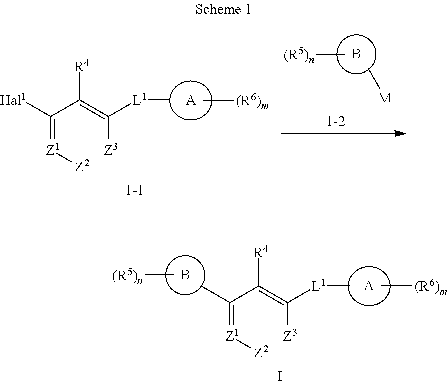

Compounds of formula II can be synthesized using a process shown in Scheme 2. In Scheme 2, a suitable halo (Hal¹)-substituted phenol 2-1 can react with a coupling reagent 2-2 (where M is, e.g., —B(OR)₂) to provide the product of formula 2-3 under standard metal catalyzed cross-coupling reaction conditions (such as Suzuki coupling reaction, e.g., in the presence of a palladium catalyst (e.g., [1,1′-bis(diphenylphosphino)ferrocene]dichloropalladium(II)) and a base (e.g., a bicarbonate or a carbonate base)). Then phenol 2-3 can react with a suitable halo (Hal²)-substituted heterocycle 2-4 under S_{N}Ar conditions using a base such as, but not limited to, potassium carbonate, to provide the compound of formula II. Compounds of formula II may also be obtained by cross-coupling conditions in the presence of a transition metal catalyst-ligand system ((e.g., copper iodide with 3,4,7,8-tetramethyl-1,10-phenanthroline,), and a base (e.g., potassium phosphate).

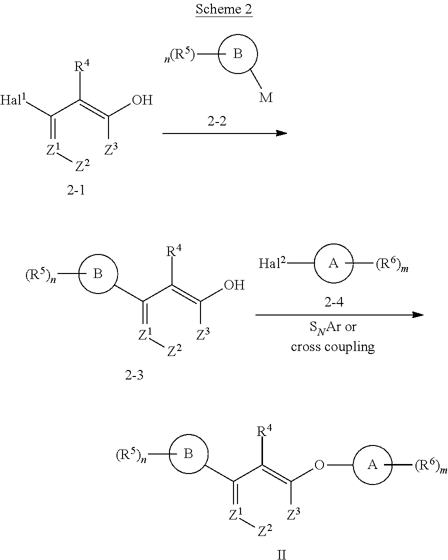

Compounds of formula III can be can be synthesized using a process shown in Scheme 3. In Scheme 3, a suitable di-halo (Hal¹, Hal²)-substituted arene 3-1 (where Hal^{I} is more reactive than Hal²) can react with a coupling reagent 3-2 (where M is, e.g., —B(OR)₂) to provide the product of formula 3-3 under standard metal catalyzed cross-coupling reaction conditions (such as Suzuki coupling reaction, e.g., in the presence of a palladium catalyst (e.g., [1,1′-bis(diphenylphosphino)ferrocene]dichloropalladium(II)) and a base (e.g., a bicarbonate or a carbonate base)). Then the aromatic halide 3-3 can react with a coupling reagent 3-4 (where M is, e.g., —B(OR)₂) to provide the product of formula III under standard metal catalyzed cross-coupling reaction conditions (such as Suzuki coupling reaction, e.g., in the presence of a palladium catalyst (e.g., [1,1′-bis(diphenylphosphino)ferrocene]dichloropalladium(II)) and a base (e.g., a bicarbonate or a carbonate base)).

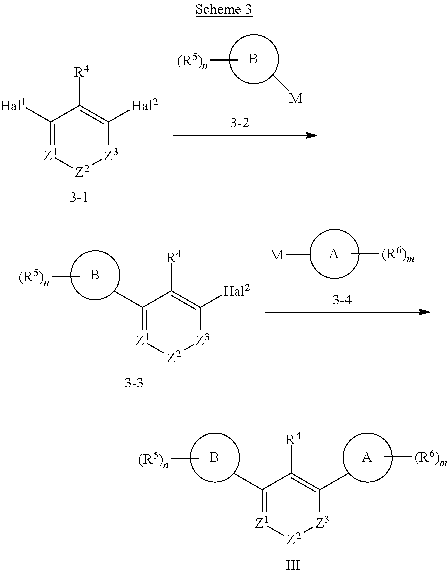

Compounds of formula III can alternatively be synthesized using a process shown in Scheme 4. A suitable halo (Hal²)-substituted arene 4-1 can be converted to a cross coupling reagent of formula 4-2 using cross-coupling reaction conditions (such as Suzuki coupling reaction, e.g., in the presence of a palladium catalyst (e.g., [1,1′-bis(diphenylphosphino)ferrocene]dichloropalladium(II)), bis(pinacolato)diboron, and a base (e.g., potassium acetate)). Alternatively, compounds of formula 4-2 may be prepared through lithium halogen exchange of halo (Hal²)-substituted arene 4-1, followed by transmetalation (e.g., reacting with trimethyl borate and quenching to provide M as —B(OH)₂). Cross coupling reagent 4-2 can react with a suitable halo (Hal³)-substituted heterocycle 4-3 to produce compounds of formula III.

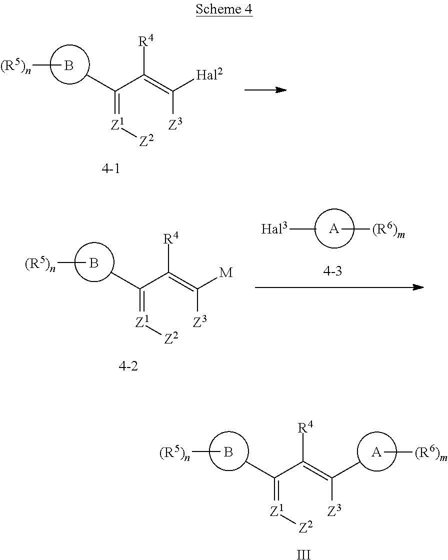

Compounds of formula IV can be synthesized using a process shown in Scheme 5. A suitable halo (Hal¹)-substituted aniline 5-1 can react with a coupling reagent 5-2 (where M is, e.g., —B(OR)₂) to provide the product of formula 5-3 under standard metal catalyzed cross-coupling reaction conditions (such as Suzuki coupling reaction, e.g., in the presence of a palladium catalyst (e.g., [1,1′-bis(diphenylphosphino)ferrocene]dichloropalladium(II)) and a base (e.g., a bicarbonate or a carbonate base)). A suitable halo (Hal²)-substituted heterocycle 5-4 can react with aniline 5-3 to produce compounds of formula IV under S_{N}Ar conditions using an acid such as, but not limited to, sulfuric acid, or base such as, but not limited to, potassium tert-butoxide. Compounds of formula IV may also be synthesized under standard metal catalyzed cross-coupling reaction conditions (such as Buchwald-Hartwig coupling reaction, e.g., in the presence of a palladium catalyst (e.g., [(4,5-bis(diphenylphosphino)-9,9-dimethylxanthene)-2-(2′-amino-1,1′-biphenyl)]palladium(II) methanesulfonate) and a base (e.g., cesium carbonate)).

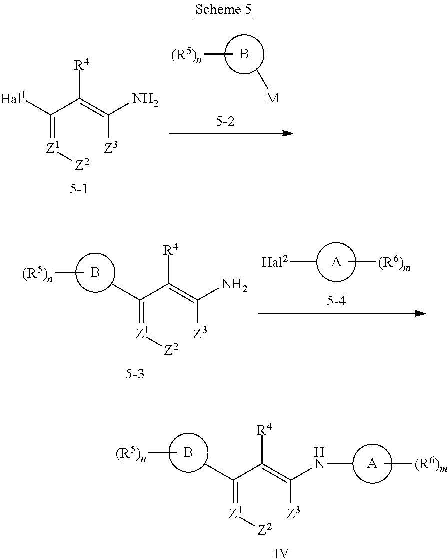

Compounds of formula V can be synthesized using a process shown in Scheme 6. A suitable benzylic alcohol 6-1 can be oxidized to an aldehyde of formula 6-2 using reagents such as, but not limited to, Dess-Martin periodinane. A compound of formula 6-2 may then be reacted with a suitable suitable halo (Hal¹)-substituted Wittig salt 6-3 (where X⁻ is, e.g., Br⁻) under standard Wittig conditions using a base such as, but not limited to, potassium tert-butoxide, to provide a compound of formula 6-4. Compounds of formula 6-4 can be converted to a cross coupling reagent of formula 6-5 using cross-coupling reaction conditions (such as Suzuki coupling reaction, e.g., in the presence of a palladium catalyst (e.g., [1,1′-bis(diphenylphosphino)ferrocene]dichloropalladium(II)), bis(pinacolato)diboron, and a base (e.g., potassium acetate)). Alternatively, compounds of formula 6-5 may be prepared through lithium halogen exchange of halo (Hal¹)-substituted arene 6-4, followed by transmetalation (e.g., reacting with trimethyl borate and quenching to provide M as —B(OH)₂). Cross coupling reagent 6-5 can react with a suitable halo (Hal²)-substituted heterocycle 6-6 to produce compounds of formula V under standard metal catalyzed cross-coupling reaction conditions (such as Suzuki coupling reaction, e.g., in the presence of a palladium catalyst (e.g., [1,1′-bis(diphenylphosphino)ferrocene]dichloropalladium(II)) and a base (e.g., a bicarbonate or a carbonate base)).

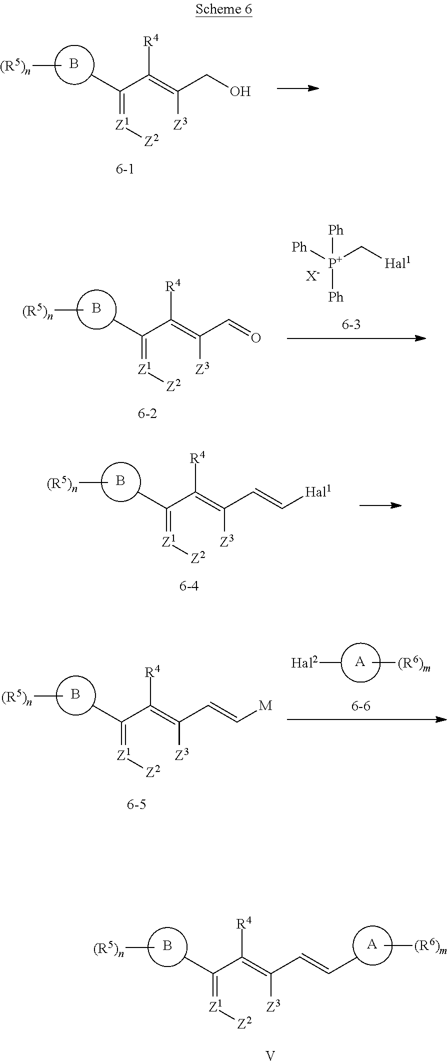

### III. Uses of the Compounds

Compounds of the present disclosure can inhibit the activity of PD-1/PD-L1 protein/protein interaction and, thus, are useful in treating diseases and disorders associated with activity of PD-1 and the diseases and disorders associated with PD-L1 including its interaction with other proteins such as PD-1 and B7-1 (CD80). Advantageously, the compounds of the present disclosure demonstrate better efficacy and favorable safety and toxicity profiles in animal studies. In certain embodiments, the compounds of the present disclosure, or pharmaceutically acceptable salts or stereoisomers thereof, are useful for therapeutic administration to enhance, stimulate and/or increase immunity in cancer, chronic infection or sepsis, including enhancement of response to vaccination. In some embodiments, the present disclosure provides a method for inhibiting or blocking the PD-1/PD-L1 protein/protein interaction. The method includes administering to an individual or a patient a compound of Formula (I) or any of the formulas as described herein or of a compound as recited in any of the claims and described herein, or a pharmaceutically acceptable salt or a stereoisomer thereof. The compounds of the present disclosure can be used alone, in combination with other agents or therapies or as an adjuvant or neoadjuvant for the treatment of diseases or disorders, including cancer or infection diseases. For the uses described herein, any of the compounds of the disclosure, including any of the embodiments thereof, may be used.

The compounds of the present disclosure inhibit the PD-1/PD-L1 protein/protein interaction, resulting in a PD-1 pathway blockade. The blockade of PD-1 can enhance the immune response to cancerous cells and infectious diseases in mammals, including humans. In some embodiments, the present disclosure provides treatment of an individual or a patient in vivo using a compound of Formula (I) or a salt or stereoisomer thereof such that growth of cancerous tumors is inhibited. A compound of Formula (I) or of any of the formulas as described herein, or a compound as recited in any of the claims and described herein, or a salt or stereoisomer thereof, can be used to inhibit the growth of cancerous tumors. Alternatively, a compound of Formula (I) or of any of the formulas as described herein, or a compound as recited in any of the claims and described herein, or a salt or stereoisomer thereof, can be used in conjunction with other agents or standard cancer treatments, as described below. In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a salt or stereoisomer thereof. In another embodiment, the present disclosure provides a method for inhibiting growth of tumor cells in an individual or a patient. The method includes administering to the individual or patient in need thereof a therapeutically effective amount of a compound of Formula (I) or of any of the formulas as described herein, or of a compound as recited in any of the claims and described herein, or a salt or a stereoisomer thereof.

In some embodiments, provided herein is a method for treating cancer. The method includes administering to a patient in need thereof, a therapeutically effective amount of a compound of Formula (I) or any of the formulas as described herein, a compound as recited in any of the claims and described herein, or a salt thereof. Examples of cancers include those whose growth may be inhibited using compounds of the disclosure and cancers typically responsive to immunotherapy.

In some embodiments, the present disclosure provides a method of enhancing, stimulating and/or increasing the immune response in a patient. The method includes administering to the patient in need thereof a therapeutically effective amount of a compound of Formula (I) or any of the formulas as described herein, a compound as recited in any of the claims and described herein, or a salt thereof.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express PD-L1.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer, lung cancer (e.g. non-small cell lung cancer and small cell lung cancer), squamous cell head and neck cancer, urothelial cancer (e.g. bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL) and multiple myeloma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

PD-1 pathway blockade with compounds of the present disclosure can also be used for treating infections such as viral, bacteria, fungus and parasite infections. The present disclosure provides a method for treating infections such as viral infections. The method includes administering to a patient in need thereof, a therapeutically effective amount of a compound of Formula (I) or any of the formulas as described herein, a compound as recited in any of the claims and described herein, a salt thereof. Examples of viruses causing infections treatable by methods of the present disclosure include, but are not limit to, human immunodeficiency virus, human papillomavirus, influenza, hepatitis A, B, C or D viruses, adenovirus, poxvirus, herpes simplex viruses, human cytomegalovirus, severe acute respiratory syndrome virus, ebola virus, and measles virus. In some embodiments, viruses causing infections treatable by methods of the present disclosure include, but are not limit to, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, comovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

The present disclosure provides a method for treating bacterial infections. The method includes administering to a patient in need thereof, a therapeutically effective amount of a compound of Formula (I) or any of the formulas as described herein, a compound as recited in any of the claims and described herein, or a salt thereof. Non-limiting examples of pathogenic bacteria causing infections treatable by methods of the disclosure include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

The present disclosure provides a method for treating fungus infections. The method includes administering to a patient in need thereof, a therapeutically effective amount of a compound of Formula (I) or any of the formulas as described herein, a compound as recited in any of the claims and described herein, or a salt thereof. Non-limiting examples of pathogenic fungi causing infections treatable by methods of the disclosure include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

The present disclosure provides a method for treating parasite infections. The method includes administering to a patient in need thereof, a therapeutically effective amount of a compound of Formula (I) or any of the formulas as described herein, a compound as recited in any of the claims and described herein, or a salt thereof. Non-limiting examples of pathogenic parasites causing infections treatable by methods of the disclosure include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the present disclosure can be used in combination with one or more other enzyme/protein/receptor inhibitors or one or more therapies for the treatment of diseases, such as cancer or infections. Examples of diseases and indications treatable with combination therapies include those as described herein. Examples of cancers include solid tumors and liquid tumors, such as blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, PI3K (alpha, beta, gamma, delta), CSFIR, KIT, FLK-II, KDR/FLK-1, FLK-4, fit-1, FGFR1, FGFR2, FGFR3, FGFR4, c-Met, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., INCB54828, INCB62079 and INCB63904), a JAK inhibitor (JAK1 and/or JAK2, e.g., ruxolitinib, baricitinib or INCB39110), an IDO inhibitor (e.g., epacadostat, NLG919, or BMS-986205), an LSD1 inhibitor (e.g., INCB59872 and INCB60003), a TDO inhibitor, a PI3K-delta inhibitor (e.g., INCB50797 and INCB50465), a PI3K-gamma inhibitor such as a PI3K-gamma selective inhibitor, a Pim inhibitor, a CSF1R inhibitor, a TAM receptor tyrosine kinases (Tyro-3, Axl, and Mer), a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), a poly ADP ribose polymerase (PARP) inhibitor such as rucaparib, olaparib, niraparib, veliparib, or talazoparib, and an adenosine receptor antagonist or combinations thereof.

Compounds of the present disclosure can be used in combination with one or more immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab. In some embodiments, the anti PD-1 antibody is SHR-1210.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab.

In some embodiments, the inhibitor of an immune checkpoint molecule is an inhibitor of LAG3, e.g., an anti-LAG3 antibody. In some embodiments, the anti-LAG3 antibody is BMS-986016, LAG525 or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an inhibitor of OX40, e.g., an anti-OX40 antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

Compounds of the present disclosure can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, Toll receptor agonists, STING agonists, oncolytic virotherapy and immunomodulating small molecules, including thalidomide or JAK1/2 inhibitor and the like. The compounds can be administered in combination with one or more anti-cancer drugs, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, olaparib, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, rucaparib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, niraparib, veliparib, talazoparib and zoledronate.

Other anti-cancer agent(s) include antibody therapeutics such as trastuzumab (Herceptin), antibodies to costimulatory molecules such as CTLA-4 (e.g., ipilimumab), 4-1BB (e.g. urelumab, utomilumab), antibodies to PD-1 and PD-L1, or antibodies to cytokines (IL-10, TGF-β, etc.). Examples of antibodies to PD-1 and/or PD-L1 that can be combined with compounds of the present disclosure for the treatment of cancer or infections such as viral, bacteria, fungus and parasite infections include, but are not limited to, nivolumab, pembrolizumab, MPDL3280A, MEDI-4736 and SHR-1210.

The compounds of the present disclosure can further be used in combination with one or more anti-inflammatory agents, steroids, immunosuppressants or therapeutic antibodies.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, ebola virus, measles virus, herpes virus (e.g., VZV, HSV-1, HAV-6, HSV-II, and CMV, Epstein Barr virus), flaviviruses, echovirus, rhinovirus, coxsackie virus, comovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

### IV. Formulation, Dosage Forms and Administration

When employed as pharmaceuticals, the compounds of the present disclosure can be administered in the form of pharmaceutical compositions. Thus the present disclosure provides a composition comprising a compound of Formula (I) or any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

The compounds of the present disclosure can further be useful in investigations of biological processes in normal and abnormal tissues. Thus, another aspect of the present invention relates to labeled compounds of the invention (radio-labeled, fluorescent-labeled, etc.) that would be useful not only in imaging techniques but also in assays, both in vitro and in vivo, for localizing and quantitating PD-1 or PD-L1 protein in tissue samples, including human, and for identifying PD-L1 ligands by inhibition binding of a labeled compound. Accordingly, the present invention includes PD-1/PD-L1 binding assays that contain such labeled compounds.

The present invention further includes isotopically-substituted compounds of the disclosure. An “isotopically-substituted” compound is a compound of the invention where one or more atoms are replaced or substituted by an atom having an atomic mass or mass number different from the atomic mass or mass number typically found in nature (i.e., naturally occurring). It is to be understood that a “radio-labeled” is a compound that has incorporated at least one one isotope that is radioactive (e.g., radionuclide). Suitable radionuclides that may be incorporated in compounds of the present invention include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro PD-L1 protein labeling and competition assays, compounds that incorporate ³H, ¹⁴C, ⁸²Br, ¹²⁵I, ¹³¹I, ³⁵S or will generally be most useful. For radio-imaging applications C, ¹⁸F, ¹²⁵I, ¹²³I, ¹²⁴I, ¹³¹I, ⁷⁵Br, ⁷⁶Br or ⁷⁷Br will generally be most useful.

In some embodiments the radionuclide is selected from the group consisting of ³H, ¹⁴C, ¹²⁵I, ³⁵S and ⁸²Br. Synthetic methods for incorporating radio-isotopes into organic compounds are known in the art.

Specifically, a labeled compound of the invention can be used in a screening assay to identify and/or evaluate compounds. For example, a newly synthesized or identified compound (i.e., test compound) which is labeled can be evaluated for its ability to bind a PD-L1 protein by monitoring its concentration variation when contacting with the PD-L1 protein, through tracking of the labeling. For example, a test compound (labeled) can be evaluated for its ability to reduce binding of another compound which is known to bind to a PD-L1 protein (i.e., standard compound). Accordingly, the ability of a test compound to compete with the standard compound for binding to the PD-L1 protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of PD-L1 including its interaction with other proteins such as PD-1 and B7-1 (CD80), such as cancer or infections, which include one or more containers containing a pharmaceutical composition comprising a therapeutically effective amount of a compound of Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of PD-1/PD-L1 protein/protein interaction according to at least one assay described herein.

### EXAMPLES

Experimental procedures for compounds of the invention are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al., “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

### Example 1

### 2-((4-(2-methylbiphenyl-3-yloxy)thieno[3,2-d]pyrimidin-7-yl)methylamino)ethanol

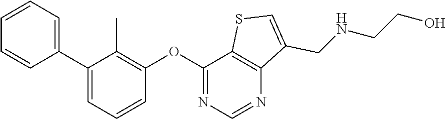 [SMILES: Cc1c(Oc2ncnc3c(CNCCO)csc23)cccc1-c1ccccc1]

### Step 1: 2-methylbiphenyl-3-ol

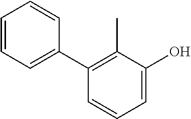

Tetrakis(triphenylphosphine)palladium(0) (300 mg, 0.3 mmol) was added to a mixture of 3-bromo-2-methylphenol (1.0 g, 5.3 mmol), phenylboronic acid (600 mg, 5 mmol), 1,4-dioxane (400 mmol) and water (200 mmol). The mixture was sparged with nitrogen for 1 min, then the mixture was sealed and stirred at 100° C. for 2 h. After cooling and concentrating the mixture in vacuo, the residue was dissolved in DCM and washed with brine. The organic layer was dried over MgSO₄, filtered, and concentrated to afford the desired product which was purified by column chromatography (0→30% EtOAc/hexanes). LCMS calculated for C₁₃H₁₃O (M+H)⁺: m/z=185.1; found 185.1.

### Step 2: 7-bromo-4-(2-methylbiphenyl-3-yloxy)thieno[3,2-d]pyrimidine

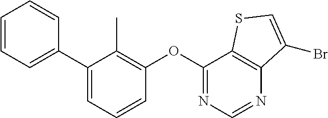 [SMILES: Cc1c(Oc2ncnc3c(Br)csc23)cccc1-c1ccccc1]

To a mixture of 7-bromo-4-chlorothieno[3,2-d]pyrimidine (Astatech, cat#SC2091: 253 mg, 1.02 mmol), potassium carbonate (280 mg, 2.03 mmol), and N,N-dimethylformamide (60 mmol) was added 2-methylbiphenyl-3-ol (224 mg, 1.22 mmol). The resulting mixture was heated to 100° C. for 1 h. After cooling, the mixture was diluted with water and ethyl acetate. The layers were separated and the organic layer was washed with water (2×5 mL) then brine. The organic layer was dried over sodium sulfate, filtered, and concentrated in vacuo. The crude product was used directly in the next step without further purification. LC-MS calculated for C₁₉H₁₄BrN₂OS (M+H)⁺: m/z=397.0; found 397.0.

### Step 3: 4-[(2-methylbiphenyl-3-yl)oxy]-7-vinylthieno[3,2-d]pyrimidine

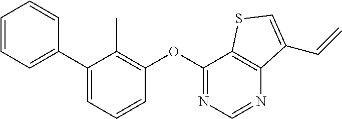 [SMILES: C=Cc1csc2c(Oc3cccc(-c4ccccc4)c3C)ncnc12]

A mixture of 7-bromo-N-(2-methylbiphenyl-3-yl)thieno[3,2-d]pyrimidin-4-amine (202 mg, 0.509 mmol), sodium carbonate (108 mg, 1.02 mmol), 4,4,5,5-tetramethyl-2-vinyl-1,3,2-dioxaborolane (0.173 mL, 1.02 mmol), and bis(di-cyclohexylphosphino)ferrocene]dichloropalladium(II) (3.8 mg, 0.0051 mmol) in tert-butyl alcohol (3.66 mL) and water (4 mL) was purged with nitrogen and sealed. The resulting mixture was stirred at 110° C. for 4 h. The reaction mixture was cooled then extracted with ethyl acetate (3×20 mL). The combined organic layers were concentrated in vacuo. The crude product was used directly in the next step without further purification. LC-MS calculated for C₂₁H₁₇N₂OS (M+1)⁺: m/z=345.1; found 345.1.

### Step 4: 4-(2-methylbiphenyl-3-yloxy)thieno[3,2-d]pyrimidine-7-carbaldehyde

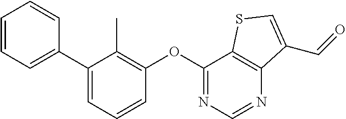 [SMILES: Cc1c(Oc2ncnc3c(C=O)csc23)cccc1-c1ccccc1]

4-[(2-methylbiphenyl-3-yl)oxy]-7-vinylthieno[3,2-d]pyrimidine (175 mg, 0.509 mmol) was dissolved in 1,4-dioxane (11 mL) and water (11 mL). To this mixture was added 4% osmium tetraoxide in water (0.48 mL, 0.076 mmol). After stirring for 5 min, sodium periodate (435 mg, 2.04 mmol) was added and the resulting mixture was stirred for 3 h. The mixture was diluted with ethyl acetate, and the layers were separated. The aqueous layer was further extracted with ethyl acetate. The combined organic layers were washed with brine, dried over MgSO₄, filtered, and concentrated in vacuo. The crude aldehyde was purified by silica gel chromatography (0→50% EtOAc/hexanes). LC-MS calculated for C₂₀H₁₅N₂O₂S (M+H)⁺: m/z=347.1; found 374.2.

### Step 5: 2-((4-(2-methylbiphenyl-3-yloxy)thieno[3,2-d]pyrimidin-7-yl)methylamino ethanol

A mixture of 4-[(2-methylbiphenyl-3-yl)oxy]thieno[3,2-d]pyrimidine-7-carbaldehyde (40 mg, 0.12 mmol) and ethanolamine (Aldrich, cat#398136: 0.021 mL, 0.35 mmol) in methylene chloride (1 mL) and N,N-diisopropylethylamine (0.120 mL, 0.69 mmol) was stirred at room temperature for 1 h. Sodium triacetoxyborohydride (0.073 g, 0.35 mmol) was carefully added in portions. The reaction was stirred at room temperature for 24 h. The mixture was diluted in methanol and purified by prep HPLC (pH=2, acetonitrile/water+TFA) to provide the desired compound as the TFA salt. LC-MS calculated for C₂₂H₂₂N₃O₂S (M+H)⁺: m/z=392.1; found 392.1.

### Example 2

### 4-(2-methylbiphenyl-3-yl)-1-[(2S)-pyrrolidin-2-ylmethyl]-1H-pyrazole

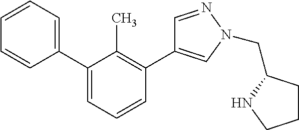 [SMILES: Cc1c(-c2ccccc2)cccc1-c1cnn(C[C@@H]2CCCN2)c1]

### Step 1: 3-bromo-2-methylbiphenyl

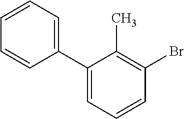

To a solution of 1-bromo-3-iodo-2-methylbenzene (800 mg, 2.70 mmol) (Oakwood, cat#037475), phenylboronic acid (344 mg, 2.83 mmol) (Aldrich, cat#78181) and sodium carbonate (712 mg, 6.72 mmol) in tert-butyl alcohol (12 mL) and water (4 mL) was added dichloro[1,1′-bis(dicyclohexylphosphino)ferrocene]palladium(II) (204 mg, 267 μmol). The reaction mixture was purged with N₂, and then heated at 90° C. for 2 h. The reaction mixture was diluted with methylene chloride, washed with saturated NaHCO₃, water and brine. The organic layer was dried over Na₂SO₄, filtered and concentrated. The residue was purified by flash chromatography on a silica gel column eluting with 10 to 20% ethyl acetate in hexanes to give the desired product (520 mg, 61%).

### Step 2: 4-(2-methylbiphenyl-3-yl)-1H-pyrazole

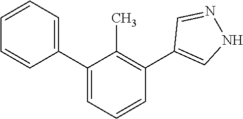 [SMILES: Cc1c(-c2ccccc2)cccc1-c1cn[nH]c1]

To a solution of 3-bromo-2-methylbiphenyl (500 mg, 2 mmol), 4-(4,4,5,5-tetramethyl-1,3,2-dioxaborolan-2-yl)-1H-pyrazole (Acros, cat#382940050: 471 mg, 2.43 mmol) and sodium carbonate (536 mg, 5.06 mmol) in tert-butyl alcohol (10 mL) and water (4 mL) was added dichloro[1,1′-bis(dicyclohexylphosphino)ferrocene]palladium(II) (153 mg, 0.202 mmol). The mixture was purged with N₂, and then heated at 110° C. for 2 h. The reaction mixture was diluted with methylene chloride, washed with saturated NaHCO₃, water and brine. The organic layer was dried over Na₂SO₄, filtered and concentrated. The residue was purified by flash chromatography on a silica gel column eluting with 30 to 60% ethyl acetate in hexanes to give the desired product (308 mg, 60%). LC-MS calculated for C₁₆H₁₅N₂ (M+H)⁺: m/z=235.2; found 235.2.

### Step 3: tert-butyl (2S)-2-{[(methylsulfonyl)oxy]methyl}pyrrolidine-1-carboxylate

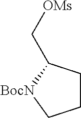 [SMILES: CC(C)(C)OC(=O)N1CCC[C@H]1COS(C)(=O)=O]

To a solution of tert-butyl (2S)-2-(hydroxymethyl)pyrrolidine-1-carboxylate (Aldrich, cat#446327: 34.4 mg, 0.171 mmol) and triethylamine (0.512 mmol) in methylene chloride (1 mL) was added methanesulfonyl chloride (19.8 μL, 0.256 mmol) at 0° C. The resulting mixture was stirred at room temperature for 1 h then diluted with EtOAc and washed with sat'd NaHCO₃. The organic layer was dried over Na₂SO₄, filtered and concentrated. The residue was used in the next step without further purification.

### Step 4: tert-butyl (2S)-2-{[4-(2-methylbiphenyl-3-yl)-1H-pyrazol-1-yl]methyl}pyrrolidine-1-carboxylate

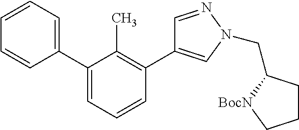 [SMILES: Cc1c(-c2ccccc2)cccc1-c1cnn(C[C@@H]2CCCN2C(=O)OC(C)(C)C)c1]

To a solution of 4-(2-methylbiphenyl-3-yl)-1H-pyrazole (20.0 mg, 0.0854 mmol) and the crude product from Step 3 in acetonitrile (0.5 mL) was added cesium carbonate (139 mg, 0.427 mmol). The reaction mixture was heated at 100° C. for 16 h, then diluted with water and extracted with methylene chloride. The combined extracts were washed with water and brine then dried over Na₂SO₄, filtered and concentrated under reduced pressure. The residue was used in the next step without further purification. LC-MS calculated for C₂₆H₃₂N₃O₂ (M+H)⁺: m/z=418.2; found 418.1.

### Step 5: 4-(2-methylbiphenyl-3-yl)-1-[(2S)-pyrrolidin-2-ylmethyl]-1H-pyrazole

The crude product from Step 4 was dissolved in methylene chloride (0.6 mL) then treated with TFA (0.3 mL). The resulting mixture was stirred at room temperature for 30 min before concentrated and purified by prep-HPLC (pH=2, acetonitrile/water+TFA) to give the desired product as the TFA salt. LC-MS calculated for C₂₁H₂₄N₃(M+H)⁺: m/z=318.2; found 318.2.

### Example 3

### 2-((2-(2-chloro-2′-fluoro-3′-methoxybiphenyl-3-ylamino)-3-fluoropyridin-4-yl)methylamino)ethanol

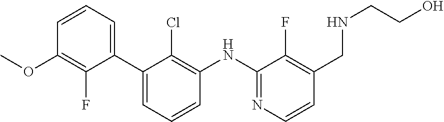 [SMILES: COc1cccc(-c2cccc(Nc3nccc(CNCCO)c3F)c2Cl)c1F]

### Step 1: 2-chloro-2′-fluoro-3′-methoxybiphenyl-3-amine

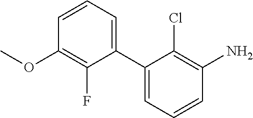 [SMILES: COc1cccc(-c2cccc(N)c2Cl)c1F]

A mixture of 3-bromo-2-chloroaniline (Ark Pharm, cat#AK156407: 700 mg, 3.39 mmol), (2-fluoro-3-methoxyphenyl)boronic acid (Combi-Blocks, cat#BB-2460: 576 mg, 3.39 mmol), [1,1′-bis(dicyclohexylphosphino)ferrocene]dichloropalladium(II) (20 mg, 0.027 mmol) (Sigma-Aldrich, cat#701998), Na₂CO₃ (1.17 g, 8.48 mmol) in dioxane (18.8 mL) and water (6.1 mL) was sparged with nitrogen. The reaction mixture was heated to 90° C. for 2 h with stirring. After cooling to room temperature, the reaction mixture was diluted with water, and extracted with EtOAc. The combined organic layers were washed with brine, dried over magnesium sulfate, filtered, and concentrated under reduced pressure. The residue was purified by flash chromatography on a silica gel column eluting with 0 to 15% methanol in dichloromethane to afford the desired product (791 mg, 93%). LCMS calculated for C₁₃H₁₂ClFNO (M+H)⁺: m/z=252.1; found 252.1.

### Step 2: 2-(2-chloro-2′-fluoro-3′-methoxybiphenyl-3-ylamino)-3-fluoroisonicotinaldehyde

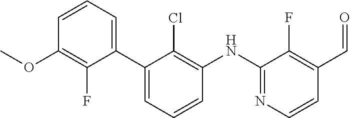 [SMILES: COc1cccc(-c2cccc(Nc3nccc(C=O)c3F)c2Cl)c1F]

A mixture of 2-chloro-2′-fluoro-3′-methoxybiphenyl-3-amine (150 mg, 0.596 mmol), 2-bromo-3-fluoroisonicotinaldehyde (Combi-Blocks, cat#QC-5746: 122 mg, 0.596 mmol), XantPhos Pd G3 (56.5 mg, 0.060 mmol), cesium carbonate (388 mg, 1.192 mmol), and 1,4-dioxane (12 mL) was sparged with nitrogen. The reaction mixture was heated to 90° C. for 3 h with stirring. After cooling to room temperature, the reaction mixture was diluted with water, and extracted with ethyl acetate. The combined organic layers were washed with brine, dried over magnesium sulfate, filtered through a pad of silica gel. The crude product was used directly in the next step without further purification. LCMS calculated for C₁₉H₁₄ClF₂N₂O₂ (M+H)⁺: m/z=375.1; found 375.2.

### Step 3: 2-((2-(2-chloro-2′-fluoro-3′-methoxybiphenyl-3-ylamino)-3-fluoropyridin-4-yl)methylamino)ethanol

A mixture of 2-(2-chloro-2′-fluoro-3′-methoxybiphenyl-3-ylamino)-3-fluoroisonicotinaldehyde (20 mg, 0.053 mmol), ethanolamine (12 μL, 0.16 mmol), N,N-diisopropylethylamine (0.046 mL, 0.267 mmol), 1,2-dichloroethane (0.5 mL) and methanol (0.15 mL) was heated to 50° C. for 3 hours. Sodium borohydride (6 mg, 0.16 mmol) was added to the mixture. The reaction mixture was allowed to stir until gas evolution ceased. The reaction was cooled to room temperature, diluted with methanol, passed through a syringe filter and purified on prep-HPLC (pH=2, acetonitrile/water+TFA) to give the desired product as the TFA salt. LCMS calculated for C₂₁H₂₁ClF₂N₃O₂ (M+H)⁺: m/z=420.1; found 420.3.

### Example 4

### (S)-1-((4-methyl-6-(2-methylbiphenyl-3-yl)quinolin-2-yl)methyl)piperidine-2-carboxylic acid

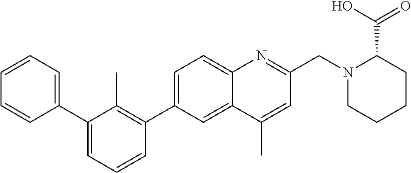 [SMILES: Cc1c(-c2ccccc2)cccc1-c1ccc2nc(CN3CCCC[C@H]3C(=O)O)cc(C)c2c1]

### Step 1: 4,4,5,5-tetramethyl-2-(2-methylbiphenyl-3-yl)-1,3,2-dioxaborolane

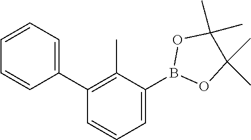 [SMILES: Cc1c(B2OC(C)(C)C(C)(C)O2)cccc1-c1ccccc1]

A mixture of 3-bromo-2-methylbiphenyl (Example 2, Step 1: 0.85 g, 3.4 mmol), potassium acetate (0.84 g, 8.6 mmol), 4,4,5,5,4′,4′,5′,5′-Octamethyl-[2,2′]bi[[1,3,2]dioxaborolanyl] (Aldrich, cat#473294: 1.0 g, 4.1 mmol), and [1,1′-is(diphenylphosphino)ferrocene]dichloropalladium(II), complex with dichloromethane (1:1) (100 mg, 0.2 mmol) in 1,4-dioxane was degassed for 5 min. The vessel was sealed and the reaction mixture was stirred at 100° C. for 2 hours. After cooling to rt, the reaction mixture was concentrated and purified by flash chromatography.

### Step 2: 2-chloro-4-methyl-6-(2-methylbiphenyl-3-yl)quinoline

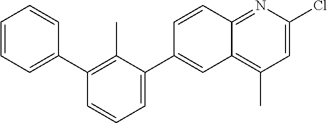 [SMILES: Cc1c(-c2ccccc2)cccc1-c1ccc2nc(Cl)cc(C)c2c1]

A mixture of 6-bromo-2-chloro-4-methylquinoline (Combi-Blocks, cat#HC-6774: 0.20 g, 0.78 mmol), 4,4,5,5-tetramethyl-2-(2-methylbiphenyl-3-yl)-1,3,2-dioxaborolane (0.25 g, 0.86 mmol), tetrakis(triphenylphosphine)palladium(0) (90. mg, 0.078 mmol) and cesium carbonate (0.51 g, 1.6 mmol) in 1,4-dioxane and water in a reaction vial was degassed and sealed. It was stirred at 80° C. for 2 hours. After cooling to rt, the reaction mixture was concentrated and purified by flash column chromatography (eluted with 0 to 30% ethyl acetate/hexane) to give a mixture (190 mg) of the desired product with the by-product 6-bromo-4-methyl-2-(2-methylbiphenyl-3-yl)quinolone, which was used in the next step without further purification.

### Step 3: 4-methyl-6-(2-methylbiphenyl-3-yl)-2-vinylquinoline

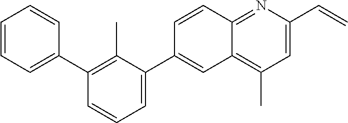 [SMILES: C=Cc1cc(C)c2cc(-c3cccc(-c4ccccc4)c3C)ccc2n1]

A mixture of 2-chloro-4-methyl-6-(2-methylbiphenyl-3-yl)quinoline and 6-bromo-4-methyl-2-(2-methylbiphenyl-3-yl)quinolone (190 mg, 0.49 mmol) from Step 1, cesium carbonate (320 mg, 0.98 mmol), 4,4,5,5-tetramethyl-2-vinyl-1,3,2-dioxaborolane (Aldrich, cat#633348: 0.73 mmol), and tetrakis(triphenylphosphine)palladium(0) (56 mg, 0.049 mmol) in 1,4-dioxane and water in a reaction vial was degassed and sealed. The reaction mixture was stirred at 80° C. for 20 hours. After cooling to rt, the reaction mixture was concentrated and purified by flash chromatography (eluted with 0 to 30% ethyl acetate/hexane) to give a mixture of the desired product with the isomer 4-methyl-2-(2-methylbiphenyl-3-yl)-6-vinylquinoline, which was used in the next step without further purification.

### Step 4: 4-methyl-6-(2-methylbiphenyl-3-yl)quinoline-2-carbaldehyde

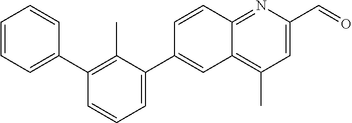 [SMILES: Cc1c(-c2ccccc2)cccc1-c1ccc2nc(C=O)cc(C)c2c1]

To a mixture of 4-methyl-2-(2-methylbiphenyl-3-yl)-6-vinylquinoline and 4-methyl-6-(2-methylbiphenyl-3-yl)-2-vinylquinoline (130 mg, 0.39 mmol) dissolved in 1,4-dioxane (3 ml) and water (1 ml) was added a 15% w/w osmium tetraoxide in water solution (0.38 ml, 0.060 mmol) at room temperature. The mixture was stirred for 5 min then sodium periodate (0.33 g, 1.5 mmol) was added. The reaction mixture was stirred at rt overnight. The reaction mixture was diluted with water and extracted with EtOAc twice. The combined extracts were washed with water and brine, dried over Na₂SO₄, and filtered. The filtrate was concentrated under reduced pressure. The residue was purified by flash chromatography. Two compounds were separated and obtained. The minor product was the desired compound and the structure was confirmed in the final step. LCMS calculated for C₂₄H₂₀NO (M+H)⁺: m/z=338.2; found 338.0.

### Step 5: (S)-1-((4-methyl-6-(2-methylbiphenyl-3-yl)quinolin-2-yl)methyl)piperidine-2-carboxylic acid

A mixture of 4-methyl-6-(2-methylbiphenyl-3-yl)quinoline-2-carbaldehyde (10 mg, 0.03 mmol) and (2S)-piperidine-2-carboxylic acid (10 mg, 0.09 mmol) in methylene chloride (1 ml) and acetic acid (0.1 mmol) was stirred at rt for 2 hours. To the mixture was then added sodium triacetoxyborohydride (19 mg, 0.089 mmol). The resulting mixture was stirred at rt overnight. The solvent was removed. The residue was dissolved in methanol, purified by prep-HPLC (pH=2, acetonitrile/water+TFA) to give the desired product as the TFA salt. LCMS calculated for C₃₀H₃₁N₂O₂ (M+H)⁺: m/z=451.2; found 451.2. ¹H NMR (500 MHz, DMSO) δ 8.13-8.07 (m, 2H), 7.88 (dd, J=8.6, 1.9 Hz, 1H), 7.53 (s, 1H), 7.50-7.33 (m, 7H), 7.28 (dd, J=7.3, 1.6 Hz, 1H), 4.76-4.45 (m, 2H), 4.21-4.08 (m, 1H), 3.53-3.43 (m, 1H), 3.18-3.05 (m, 1H), 2.76 (s, 3H), 2.19-2.13 (m, 1H), 2.11 (s, 3H), 1.95-1.83 (m, 1H), 1.80-1.71 (m, 2H), 1.69-1.49 (m, 2H).

### Example 5

### 5-(2-methylbiphenyl-3-yl)isoindoline

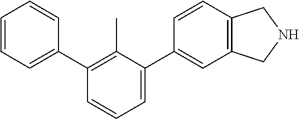 [SMILES: Cc1c(-c2ccccc2)cccc1-c1ccc2c(c1)CNC2]

### Step 1: tert-butyl 5-(2-methylbiphenyl-3-yl)isoindoline-2-carboxylate

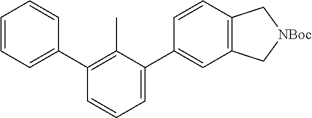 [SMILES: Cc1c(-c2ccccc2)cccc1-c1ccc2c(c1)CN(C(=O)OC(C)(C)C)C2]

A mixture of 3-bromo-2-methylbiphenyl (Example 2, Step 1: 40 mg, 0.2 mmol), tert-butyl 5-(4,4,5,5-tetramethyl-1,3,2-dioxaborolan-2-yl)-1,3-dihydro-2H-isoindole-2-carboxylate (67 mg, 0.19 mmol), [1,1′-bis(diphenylphosphino)ferrocene]dichloropalladium(II) dichloromethane complex (1:1) (7 mg, 0.008 mmol) and potassium carbonate (67 mg, 0.48 mmol) in 1,4-dioxane (20 mL) and water (1 mL) was degassed and recharged with nitrogen three times. The mixture was then heated and stirred at 120° C. overnight. The reaction mixture was quenched with water, and extracted with ethyl acetate (3×10 mL). The combined organic layers were washed with brine, dried over MgSO₄, filtered and concentrated under reduced pressure to afford the crude product.

### Step 2: 5-(2-methylbiphenyl-3-yl)isoindoline

TFA (1 mL) was added to the solution of the crude product from Step 1 in DCM (1 mL). The reaction mixture was stirred at room temperature for 1 h. The reaction mixture was concentrated under reduced pressure. The residue was purified by prep-HPLC (pH=2, acetonitrile/water+TFA) to afford the desired product as TFA salt. LCMS calculated for C₂₁H₂₀N (M+H)⁺: m/z=286.2; found 286.2.

### Example 6

### (5-(2-chlorobiphenyl-3-yl)pyridin-2-yl)methanamine

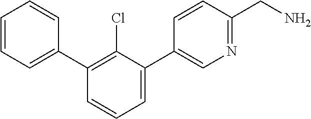 [SMILES: NCc1ccc(-c2cccc(-c3ccccc3)c2Cl)cn1]

### Step 1: 5-(2-chlorobiphenyl-3-yl)picolinamide

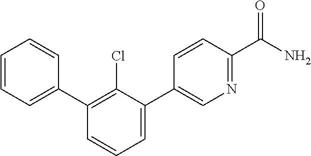 [SMILES: NC(=O)c1ccc(-c2cccc(-c3ccccc3)c2Cl)cn1]

A mixture of 1,3-dibromo-2-chlorobenzene (Combi-Blocks, cat#QA2717: 200 mg, 0.74 mmol), phenylboronic acid (95 mg, 0.78 mmol), [1,1′-bis(diphenylphosphino)ferrocene]dichloropalladium(II) dichloromethane complex (1:1) (30 mg, 0.04 mmol) and potassium carbonate (0.51 g, 3.7 mmol) in 1,4-dioxane (10 mL) and water (5 mL) was degassed and recharged with nitrogen three times. The mixture was then heated and stirred at 80° C. for 4 h. The reaction mixture was cooled to room temperature and then 5-(4,4,5,5-tetramethyl-1,3,2-dioxaborolan-2-yl)pyridine-2-carbonitrile (Combi-Blocks, cat#PN-8873: 0.17 g, 0.74 mmol) was added. The mixture was then stirred at 110° C. for 4 h. The reaction mixture was cooled to room temperature, quenched with water, and extracted with ethyl acetate (3×10 mL). The combined organic layers were washed with brine, dried over Na₂SO₄, filtered and concentrated under reduced pressure. The residue was purified by prep-HPLC (pH=2, acetonitrile/water+TFA) to afford the desired product as the TFA salt. LCMS calculated for C₁₈H₁₄ClN₂O (M+H)⁺: m/z=309.1; found 309.1.

### Step 2: (5-(2-chlorobiphenyl-3-yl)pyridin-2-yl)methanamine

1.0M Lithium tetrahydroaluminate in THF (1.0 mL) was added to a mixture of 5-(2-chlorobiphenyl-3-yl)pyridine-2-carboxamide (50 mg, 0.2 mmol) in THF (1 mL). The reaction mixture was stirred at room temperature overnight. The reaction mixture was quenched with saturated aqueous NH₄Cl, and extracted with ethyl acetate (3×20 mL). The combined organic layers were washed with brine, dried over MgSO₄, filtered and concentrated under reduced pressure. The residue was purified by prep-HPLC (pH=2, acetonitrile/water+TFA) to afford the desired product as TFA salt. LCMS calculated for C₁₈H₁₆ClN₂ (M+H)⁺: m/z=295.1; found 295.1.

### Example 7

### 2-{[(2′-methyl-1,1′:3′,1″-terphenyl-4-yl)methyl]amino}ethanol

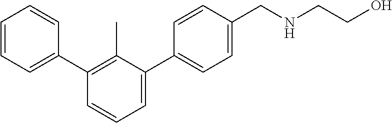 [SMILES: Cc1c(-c2ccccc2)cccc1-c1ccc(CNCCO)cc1]

### Step 1: 2′-methyl-1,1′:3′,1″-terphenyl-4-carbaldehyde

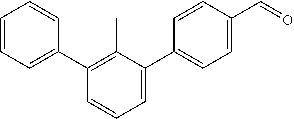 [SMILES: Cc1c(-c2ccccc2)cccc1-c1ccc(C=O)cc1]

A mixture of 3-bromo-2-methylbiphenyl (Example 2, Step 1: 201 mg, 0.813 mmol), 4-formylphenylboronic acid (Aldrich, cat#431966: 130 mg, 0.89 mmol), [1,1′-bis(diphenylphosphino)ferrocene]dichloropalladium(II) dichloromethane complex (1:1) (30 mg, 0.04 mmol) and potassium carbonate (340 mg, 2.4 mmol) in 1,4-dioxane (10 mL) and water (5 mL) was degassed and recharged with nitrogen three times. The mixture was then heated and stirred at 110° C. overnight. The reaction mixture was quenched with water, and extracted with ethyl acetate (3×10 mL). The combined organic layers were washed with brine, dried over Na₂SO₄, filtered and concentrated under reduced pressure to afford the crude product, which was used in the next step without further purification.

### Step 2: 2-{[(2′-methyl-1,1′:3′,1″-terphenyl-4-yl)methyl]amino}ethanol

Ethanolamine (5.4 uL, 0.082 mmol) was added to a solution of 2′-methyl-1,1′:3′,1″-terphenyl-4-carbaldehyde (15 mg, 0.055 mmol) in N,N-dimethylformamide (0.5 mL), followed by acetic acid (9.4 uL, 0.16 mmol). After 5 min, sodium cyanoborohydride (10.0 mg, 0.16 mmol) was added. The reaction mixture was stirred at room temperature overnight. The mixture was purified by prep-HPLC (pH=2, acetonitrile/water+TFA) to afford the desired product as the TFA salt. LCMS calculated for C₂₂H₂₄NO (M+H)⁺: m/z=318.2; found 318.2.

### Example 8

### (R,E)-1-((6-(2-(2-methyl-[1,1′-biphenyl]-3-yl)vinyl)pyridin-3-yl)methyl)piperidine-2-carboxylic acid

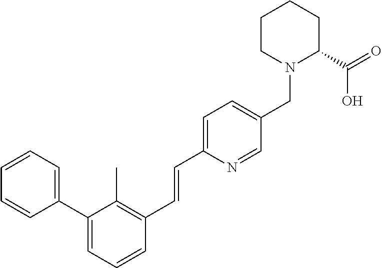 [SMILES: Cc1c(/C=C/c2ccc(CN3CCCC[C@@H]3C(=O)O)cn2)cccc1-c1ccccc1]

### Step 1: 2-methyl-[1,1′-biphenyl]-3-carbaldehyde

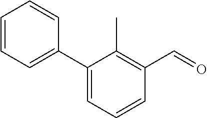

Dess-Martin periodinane (8.32 mmol) was added to a solution of (2-methyl-[1,1′-biphenyl]-3-yl)methanol (TCI, cat#H0777:1.5 g, 7.57 mmol) in methylene chloride (16.00 ml, 250 mmol). After 0.5 h, saturated aqueous NaHCO₃ was added. After stirring for 0.5 h, the mixture was filtered. The organic layer was washed with saturated aqueous NaHCO₃, 10% w/w aqueous Na₂CO₃, and then brine. The organic layer was dried over Na₂SO₄, filtered and concentrated under reduced pressure to provide the desired aldehyde as a pale orange paste. The crude material was purified by silica gel chromatography (eluting with 0-15% ethyl acetate in hexanes) providing a colorless oil which was triturated w/˜ hexanes to give 0.85 g of the desired product as a white crystalline solid (57.2% yield). LC-MS calculated for C₁₄H₁₃O (M+H)⁺: m/z=197.1; found 197.2. ¹H NMR (400 MHz, CDCl3) δ 10.41 (s, 1H), 7.86 (dd, J=7.5, 1.3 Hz, 2H), 7.68-7.36 (m, 3H), 7.39-7.22 (m, 3H), 2.58 (s, 3H).

### Step 2: (E)-3-(2-bromovinyl)-2-methyl-1,1′-biphenyl

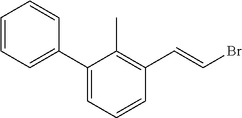 [SMILES: Cc1c(/C=C/Br)cccc1-c1ccccc1]

To a suspension of (bromomethyl)triphenylphosphonium bromide (Aldrich, cat#269158: 2.369 g, 5.43 mmol) in THF (15.93 ml, 196 mmol) at −78° C. was added 1.0M potassium tert-butoxide in THF (5.43 mmol) over 5 min, and the reaction mixture was stirred for 1 h. 2-methyl-[1,1′-biphenyl]-3-carbaldehyde (0.82 g, 4.18 mmol) was added and allowed to warm to −40° C. over 4 h. The reaction was then allowed to warm up to rt and was stirred for another 1 h. The reaction was quenched with water and then diluted with ethyl acetate. The organic layer was washed with water ×2, and saturated NaCl. The organic layer was then dried over Na₂SO₄ and rotovaped to give 1.55 g orange paste. The crude was purified by silica gel chromatography eluting with 0-5% ethyl acetate in hexanes to give 1.55 g colorless oil (74% yield). LC-MS calculated for C₁₅H₁₄Br (M+H)⁺: m/z=273.0; found 273.1. ¹H NMR (400 MHz, CDCl₃) δ 7.55 (d, J=7.5 Hz, 1H), 7.48-7.08 (m, 8H), 6.61 (d, J=7.8 Hz, 1H), 2.20 (s, 3H).

### Step 3: (E)-4,4,5,5-tetramethyl-2-(2-(2-methyl-[1,1′-biphenyl]-3-yl)vinyl)-1,3,2-dioxaborolane

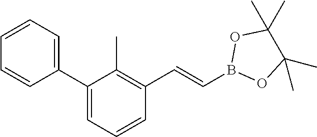 [SMILES: Cc1c(/C=C/B2OC(C)(C)C(C)(C)O2)cccc1-c1ccccc1]

To a reaction vial was combined (E)-3-(2-bromovinyl)-2-methyl-1,1′-biphenyl (0.85 g, 3.11 mmol), potassium acetate (0.584 ml, 9.33 mmol) and 4,4,4′,4′,5,5,5′,5′-octamethyl-2,2′-bi(1,3,2-dioxaborolane) (1.344 ml, 4.67 mmol). To this mixture was added 1,4-dioxane (14.08 ml, 180 mmol) and the resulting mixture was sparged with nitrogen for 5 min. [1,1′-Bis(diphenylphosphino)ferrocene]dichloropalladium(II) dichloromethane complex (1:1) (0.127 g, 0.156 mmol) was then added, and the reaction was sealed and heated to 90° C. for 4 h. The reaction was cooled, and concentrated under reduced pressure to give a black solid/glass. Ethyl acetate and water were added. The layers were separated and the organic phase was washed brine. The organic layer was dried over Na₂SO₄ and concentrated in vacuo to give 1.75 g of a black oil. The mixture was purified by silica gel chromatography (eluting with 0-10% ethyl acetate in hexanes) to give 0.66 g of the desired product as an orange oil (66% yield). LC-MS calculated for C₂₁H₂₆BO₂ (M+H)⁺: m/z=321.2; found 321.2.

### Step 4: (E)-6-(2-(2-methyl-[1,1′-biphenyl]-3-yl)vinyl)nicotinaldehyde

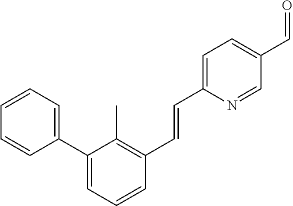 [SMILES: Cc1c(/C=C/c2ccc(C=O)cn2)cccc1-c1ccccc1]

A degased mixture of 6-bromonicotinaldehyde (Aldrich, cat#596280: 4.91 mg, 0.026 mmol), (Z)-4,4,5,5-tetramethyl-2-(2-(2-methyl-[1,1′-biphenyl]-3-yl)vinyl)-1,3,2-dioxaborolane (10.99 mg, 0.034 mmol), dichloro[1,1′-bis(dicyclohexylphosphino)ferrocene]palladium(II) (20. mg, 0.026 mmol) and potassium carbonate (6.39 μl, 0.071 mmol) in 1,4-dioxane (1 mL) and water (0.3 mL) was heated at 90° C. overnight. Ethyl acetate and water were added, and the mixture was filtered. The organic phase was washed brine, dried over Na₂SO₄, filtered, and concentrated under reduced pressure. The crude mixture was purified by silica gel chromatography eluting with 0-25% ethyl acetate in hexanes to give the desired product as a colorless oil. LC-MS calculated for C₂₁H₁₈NO (M+H)⁺: m/z=300.1; found 300.2.

### Step 5: (R,E)-1-((6-(2-(2-methyl-[1,1′-biphenyl]-3-yl) vinyl)pyridin-3-yl)methyl)piperidine-2-carboxylic acid

A mixture of (E)-6-(2-(2-methyl-[1,1′-biphenyl]-3-yl)vinyl)nicotinaldehyde (53.7 mg, 0.179 mmol), methyl (S)-piperidine-2-carboxylate hydrochloride (Combi-Blocks, cat#YC-0952: 97 mg, 0.538 mmol) and N,N-diisopropylethylamine (0.156 mL, 0.896 mmol) in methylene chloride (1 mL) was stirred for 3 h. Sodium triacetoxyborohydride (38 mg, 0.179 mmol) was added and stirred overnight. The organic phase was washed with water, aqueous saturated NaHCO₃, then brine. The organic layer was then dried over Na₂SO₄, filtered, and concentrated.

The resulting residue ((R,E)-1-((6-(2-(2-methyl-[1,1′-biphenyl]-3-yl)vinyl)pyridin-3-yl)methyl)piperidine-2-carboxylate (8.8 mg, 0.021 mmol)) was dissolved in methanol (1 mL), tetrahydrofuran (1 mL) and 1.0M sodium hydroxide in water (4.48 mmol) and then was stirred for 1.5 h. The mixture was then diluted with methanol and purified by prep-HPLC (pH=10, acetonitrile/water+NH₄OH) to provide the desired compound as a white powder. LC-MS calculated for C₂₇H₂₉N₂O₂ (M+H)⁺: m/z=413.2; found 413.2.

### Example 9

### 2-(((8-((2′-fluoro-3′-methoxy-2-methyl-[1,1′-biphenyl]-3-yl)amino)imidazo[1,2-a]pyrazin-3-yl)methyl)amino)ethan-1-ol

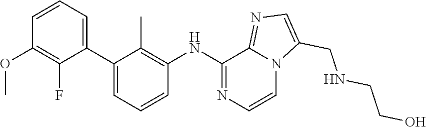 [SMILES: COc1cccc(-c2cccc(Nc3nccn4c(CNCCO)cnc34)c2C)c1F]

### Step 1: 8-chloro-3-vinylimidazo[1,2-a]pyrazine

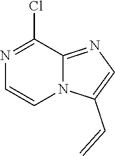

A mixture of 3-bromo-8-chloroimidazo[1,2-a]pyrazine (Alchem Pharmtech, cat#Z-01430: 1.5 g, 6.4 mmol), 4,4,5,5-tetramethyl-2-vinyl-1,3,2-dioxaborolane (Aldrich, cat#633348: 7.1 mmol), sodium carbonate (1.4 g, 13 mmol) and dichloro[1,1′-bis(dicyclohexylphosphino)ferrocene]palladium(II) (75 mg, 0.099 mmol) in 1,4-dioxane (15 mL) and water (5 mL) was degassed and heated at 97° C. overnight. Ethyl acetate and water were added and the mixture was filtered. The layers were separated and the aqueous phase was further extracted with ethyl acetate. The combined organic layers were washed with brine, dried over Na₂SO₄, filtered, and concentrated in vacuo. The crude residue was purified by silica gel chromatography eluting with 0-50% ethyl acetate in hexanes to give 0.23 g pale orange solid (20% yield). LC-MS calculated for C₈H₇ClN₃ (M+H)⁺: m/z=180.0; found 180.0.

### Step 2: (N-(3-bromo-2-methylphenyl)-3-vinylimidazo[1,2-a]pyrazin-8-amine

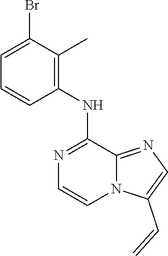 [SMILES: C=Cc1cnc2c(Nc3cccc(Br)c3C)nccn12]

A suspension of 3-bromo-2-methylaniline (Aldrich, cat#530018: 0.12 mL, 0.974 mmol), cesium carbonate (1.071 mmol) and 8-chloro-3-vinylimidazo[1,2-a]pyrazine (0.175 g, 0.974 mmol) in acetonitrile (1.679 mL, 32.1 mmol) was heated at 105° C. overnight. Ethyl acetate and water were added and the mixture was filtered. The organic layer was washed with brine, dried over Na₂SO₄, filtered, and concentrated in vacuo. The crude residue was purified by silica gel chromatography eluting with 0-25% ethyl acetate in hexanes to give 0.11 g of an off-white solid (39% yield). LC-MS calculated for C₁₅H₁₄BrN₄ (M+H)⁺: m/z=329.0; found 329.0.

### Step 3: 8-[(3-bromo-2-methylphenyl)amino]imidazo[1,2-a]pyrazine-3-carbaldehyde

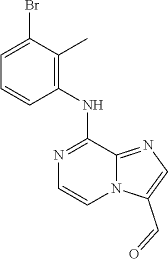 [SMILES: Cc1c(Br)cccc1Nc1nccn2c(C=O)cnc12]

To a solution N-(3-bromo-2-methylphenyl)-3-vinylimidazo[1,2-a]pyrazin-8-amine (0.11 g, 0.334 mmol) of in 1,4-dioxane (3 mL) and water (3 mL) was added 0.157M osmium tetraoxide in water (0.053 mmol). After 2 min, the reaction turned orange. Sodium metaperiodate (0.35 g, 1.6 mmol) was added and the reaction was stirred for 3 h. Ethyl acetate and water were added, and the mixture was filtered. The organic phase was washed with aqueous saturated NaHCO₃ and dried over Na₂SO₄, filtered, and concentrated in vacuo. The crude residue was purified by silica gel chromatography eluting with 0-50% ethyl acetate in hexanes to give the desired product as an orange solid. LC-MS calculated for C₁₄H₁₂BrN₄O (M+H)⁺: m/z=331.0; found 331.1.

### Step 4: 2-[({8-[(3-bromo-2-methylphenyl)amino]imidazo[1,2-a]pyrazin-3-yl}methyl)amino]ethanol

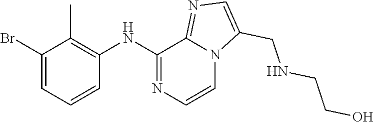 [SMILES: Cc1c(Br)cccc1Nc1nccn2c(CNCCO)cnc12]

To a suspension of 8-((3-bromo-2-methylphenyl)amino)imidazo[1,2-a]pyrazine-3-carbaldehyde (0.170 g, 0.514 mmol) in methylene chloride (4 mL) was added 2-aminoethan-1-ol (31 μL, 0.514 mmol) then acetic acid (29.2 μL, 0.514 mmol). After 1 h, sodium triacetoxyborohydride (0.272 g, 1.284 mmol) was added and stirred overnight. Water was added, and the layers were separated. The organic phase was washed aqueous saturated sodium bicarbonate, brine, dried over Na₂SO₄, filtered, and concentrated in vacuo. The resulting residue was triturated with 3 mL METB to provide the desired product as an orange solid. LC-MS calculated for C₁₆H₁₀BrN₅O (M+H)⁺: m/z=376.1; found 376.1.

### Step 5: 2-(((8-((2′-fluoro-3′-methoxy-2-methyl-[1,1′-biphenyl]-3-yl)amino)imidazo[1,2-a]pyrazin-3-yl)methyl)amino)ethan-1-ol

A mixture of 2-[({8-[(3-bromo-2-methylphenyl)amino]imidazo[1,2-a]pyrazin-3-yl}methyl)amino]ethanol (10. mg, 0.026 mmol), (2-fluoro-3-methoxyphenyl)boronic acid (Aldrich, cat#594253:8.2 mg, 0.048 mmol), sodium carbonate (8.4 mg, 0.080 mmol) and dichloro[1,1′-bis(dicyclohexylphosphino)ferrocene]palladium(II) (3.5 mg, 0.0046 mmol) in 1,4-dioxane (0.3 mL) and water (0.1 mL) was degassed and refluxed at 110° C. overnight. The mixture was diluted with methanol and purified by prep-HPLC (pH=10, acetonitrile/water+NH₄OH) to provide the desired compound as a white powder. LC-MS calculated for C₂₃H₂₅FN₅O₂ (M+H)⁺: m/z=422.2; found 422.2.

### Example 10: (2S)-1-(3-methyl-4-(4-phenyl-2,3-dihydro-1H-inden-1-yloxy)benzyl)piperidine-2-carboxylic acid

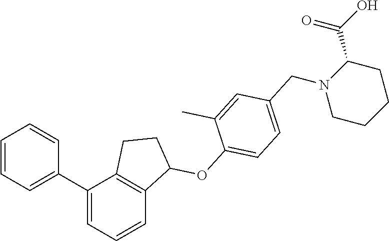 [SMILES: Cc1cc(CN2CCCC[C@H]2C(=O)O)ccc1OC1CCc2c(-c3ccccc3)cccc21]

### Step 1: 4-phenyl-2,3-dihydro-1H-inden-1-ol

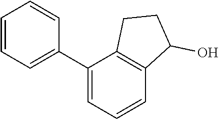 [SMILES: OC1CCc2c(-c3ccccc3)cccc21]

A mixture of 4-bromoindan-1-ol (Combi-Blocks cat#QH-683: 321 mg, 1.51 mmol), Phenylboronic acid (Aldrich cat# P20009: 220 mg, 1.81 mmol), bis(di-cyclohexylphosphino)ferrocene]dichloropalladium(II) (60 mg, 0.08 mmol) and Potassium phosphate (1300 mg, 6.0 mmol) in a mixture of Water (3 mL) and 1,4-Dioxane (20 mL) was degassed with nitrogen, then heated in a sealed vial at 90° C. 3 hours. The reaction mixture was cooled to room temperature and filtered. The filtrate was concentrated to dryness. The crude residue was purified by silica gel chromatography eluting with 20% ethyl acetate in hexanes to give desired product. LC-MS calculated for C₁₅H₁₃ (M−H₂O)⁺: m/z=193.1; 193.1:

### Step 2: (S)-methyl 1-(4-hydroxy-3-methylbenzyl)piperidine-2-carboxylate

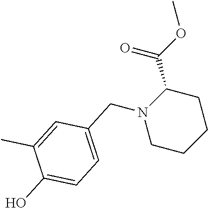 [SMILES: COC(=O)[C@@H]1CCCCN1Cc1ccc(O)c(C)c1]

To a solution of 4-hydroxy-3-methylbenzaldehyde (Aldrich cat#316911: 400.0 mg, 2.938 mmol) and N,N-diisopropylethylamine (8.8 mmol) in 1,2-dichloroethane (200 mmol) was added (S)-methyl piperidine-2-carboxylate hydrochloride (Combi-Blocks cat#SS-2950: 690 mg, 3.8 mmol) followed by sodium triacetoxyborohydride (1.9 g, 8.8 mmol). The mixture was stirred at room temperature overnight. The crude reaction mixture was diluted with DCM, then sequentially washed with an aqueous NaHCO₃ solution, water, and brine. The organic layer was dried over Na₂SO₄, filtered, and concentrated under reduced pressure. The crude residue was purified by silica gel chromatography eluting with 40% ethyl acetate in hexanes to give the desired product. LC-MS calculated for C₁₅H₂₂NO₃ (M+H)⁺: m/z=264.2; found: 264.2.

### Step 3: (2S)-methyl 1-(3-methyl-4-(4-phenyl-2,3-dihydro-1H-inden-1-yloxy)benzyl)piperidine-2-carboxylate

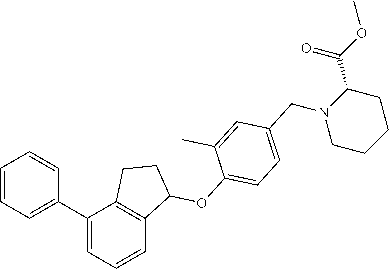 [SMILES: COC(=O)[C@@H]1CCCCN1Cc1ccc(OC2CCc3c(-c4ccccc4)cccc32)c(C)c1]

To a solution of methyl (S)-1-(4-hydroxy-3-methylbenzyl)piperidine-2-carboxylate (115 mg, 0.437 mmol), 4-phenylindan-1-ol (91.8 mg, 0.437 mmol), and triphenylphosphine (230 mg, 0.87 mmol) in toluene (5 ml) at room temperature was added diethyl azodicarboxylate (0.66 mmol) dropwise. The reaction mixture was stirred at 70° C. for 4 hours after which time the crude reaction mixture was concentrated under reduced pressure. The crude residue was purified by silica gel chromatography eluting with 20% ethyl acetate in hexanes to give the desired product. LC-MS calculated for C₃₀H₃₄NO₃ (M+H)⁺: m/z=456.3; found: 456.1.

### Step 4: (2S)-1-(3-methyl-4-(4-phenyl-2,3-dihydro-1H-inden-1-yloxy)benzyl)piperidine-2-carboxylic acid

To a mixture of (2S)-methyl 1-(3-methyl-4-(4-phenyl-2,3-dihydro-1H-inden-1-yloxy)benzyl)piperidine-2-carboxylate (18 mg, 0.040 mmol) in THF (1 ml) and Methanol (1 ml) was added lithium hydroxide hydrate (20 mg, 0.4 mmol) and water (0.4 mL). The resulting mixture was stirred at r.t. overnight. The crude reaction mixture was diluted with methanol and purified by prep-HPLC (pH=10, acetonitrile/water+NH₄OH) to provide the desired compound as a white powder. LC-MS calculated for C₂₉H₃₂NO₃ (M+H)⁺: m/z=442.2; found 442.2.

### Example 11: (S)-1-((8-(2-methylbiphenyl-3-ylcarbamoyl)imidazo[1,2-a]pyridin-3-yl)methyl)piperidine-2-carboxylic acid

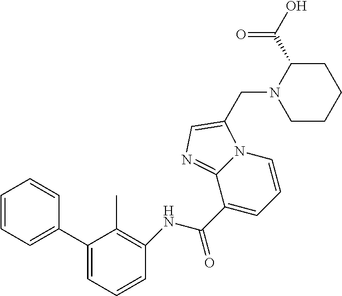 [SMILES: Cc1c(NC(=O)c2cccn3c(CN4CCCC[C@H]4C(=O)O)cnc23)cccc1-c1ccccc1]

### Step 1: 2-methylbiphenyl-3-amine

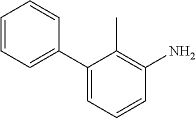

To a solution of 3-bromo-2-methylaniline (Aldrich cat#530018: 1.0 g, 5.4 mmol) in water (3 ml) and 1,4-dioxane (10 ml) was added phenylboronic acid (Aldrich cat# P20009: 0.79 g, 6.4 mmol), [1,1′-Bis(diphenylphosphino)ferrocene]dichloropalladium(II), complex with dichloromethane (1:1) (0.3 g, 0.3 mmol) and sodium carbonate (1.2 g, 11 mmol). The reaction mixture was degassed with nitrogen and then heated at 100° C. for 3 hours. The crude reaction mixture was then cooled to rt and filtered over celite. The filtrate was concentrated under reduced pressure. The crude residue was purified by silica gel chromatography eluting with 20% ethyl acetate in hexanes to give desired product. LC-MS calculated for C₁₃H₁₄N (M+H)⁺: m/z=184.1; found: 184.1.

### Step 2: methyl 3-vinylimidazo[1,2-a]pyridine-8-carboxylate

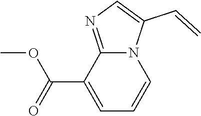

A mixture of methyl 3-bromoimidazo[1,2-a]pyridine-8-carboxylate (Combi-Blocks cat# HC-2497: 350 mg, 1.4 mmol), 4,4,5,5-tetramethyl-2-vinyl-1,3,2-dioxaborolane (2.0 mmol), [1,1′-Bis(diphenylphosphino)ferrocene]dichloropalladium(II), complex with dichloromethane (1:1) (0.07 g, 0.08 mmol) and potassium carbonate (600 mg, 4 mmol) in 1,4-dioxane (15 mL) and water (3 mL) was purged with nitrogen and then heated at 100° C. for 2 hours. The crude reaction mixture was cooled to room temperature and then filtered over celite. The filtrate was concentrated under reduced pressure. The crude residue was purified by silica gel chromatography eluting with 60% ethyl acetate in DCM to give the desired product. LC-MS calculated for C₁₁H₁₁N₂O₂(M+H)⁺: m/z=203.1; found: 203.1.

### Step 3: methyl 3-formylimidazo[1,2-a]pyridine-8-carboxylate

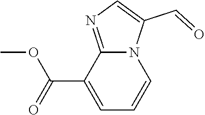

To a solution of methyl 3-vinylimidazo[1,2-a]pyridine-8-carboxylate (200.0 mg, 0.9891 mmol) in 1,4-dioxane (10 ml) and water (2 ml) was added, 2,6-lutidine (0.46 ml, 4.0 mmol), followed by a solution of osmium tetroxide in water (1 mL, 0.15 mmol). The crude reaction mixture was stirred for 10 min at room temperature after which time sodium periodate (630 mg, 3.0 mmol) as a solution in water (1.5 mL) was added. The reaction mixture was stirred at r.t. for 3 hours. The crude reaction suspension was diluted with water then extracted with EtOAc. The combined organic layers were washed with water and brine, dried over MgSO₄, filtered and concentrated under reduced pressure. The crude residue was purified by silica gel chromatography eluting with 50% ethyl acetate in DCM to give desired product. LC-MS calculated for C₁₀H₉N₂O₃ (M+H)⁺: m/z=205.1; found: 205.2.

### Step 4: 3-formylimidazo[1,2-a]pyridine-8-carboxylic acid

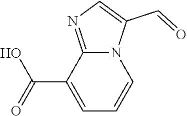 [SMILES: O=Cc1cnc2c(C(=O)O)cccn12]

To a mixture of methyl 3-formylimidazo[1,2-a]pyridine-8-carboxylate (56.0 mg, 0.274 mmol) in THF (2 ml) and methanol (1 ml) was added lithium hydroxide hydrate (46 mg, 1.1 mmol) and water (0.4 mL). The resulting mixture was stirred at room temperature for 1 hour. The crude reaction mixture was acidified with small amount 1N HCl solution, then extracted with DCM. The combined organic layers were dried over Na₂SO₄, filtered, and concentrated under reduced pressure to afford the desired compound which was taken on without further purification. LC-MS calculated for C₉H₇N₂O₃(M+H)⁺: m/z=191.0; found: 191.1.

### Step 5: 3-formyl-N-(2-methylbiphenyl-3-yl)imidazo[1,2-a]pyridine-8-carboxamide

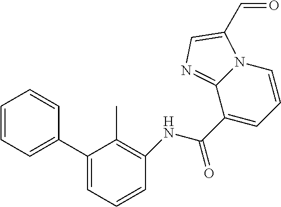 [SMILES: Cc1c(NC(=O)c2cccn3c(C=O)cnc23)cccc1-c1ccccc1]

To the solution of 3-formylimidazo[1,2-a]pyridine-8-carboxylic acid (60 mg, 0.3 mmol) and 2-methylbiphenyl-3-amine (Example 11, Step 1: 63.6 mg, 0.347 mmol) in N,N-dimethylformamide (3 ml) was added N,N,N′,N′-Tetramethyl-O-(7-azabenzotriazol-1-yl)uronium hexafluorophosphate (180 mg, 0.47 mmol), followed by N,N-diisopropylethylamine (0.95 mmol). The crude reaction mixture was stirred for 3 hours at room temperature. The crude reaction mixture was diluted with DCM, then washed sequentially with aqueous NaHCO₃ solution, water, and brine. The organic layer was dried over Na₂SO₄, filtered, and concentrated under reduced pressure. The crude residue was purified by silica gel chromatography eluting with 15% ethyl acetate in hexanes to give desired product. LC-MS calculated for C₂₂H₁₈N₃O₂ (M+H)⁺: m/z=356.1; found: 356.1.

### Step 6: (S)-1-((8-(2-methylbiphenyl-3-ylcarbamoyl) imidazof[1,2-a]pyridin-3-yl)methyl)piperidine-2-carboxylic acid

To a solution of 3-formyl-N-(2-methylbiphenyl-3-yl)imidazo[1,2-a]pyridine-8-carboxamide (10.0 mg, 0.028 mmol) in 1,2-dichloroethane (1 ml) was added acetic acid (0.14 mmol) and (2S)-piperidine-2-carboxylic acid (Aldrich cat#P2519: 7.3 mg, 0.056 mmol). The mixture was allowed to stir at room temperature for 20 min after which time sodium triacetoxyborohydride (18 mg, 0.084 mmol) was added and was allowed to stir overnight. The crude reaction mixture was concentrated to dryness under reduced pressure and the residue was dissolved with MeOH, and purified by prep-HPLC (pH=10, acetonitrile/water+NH₄OH) to provide the desired compound as a white powder. LC-MS calculated for C₂₈H₂₉N₄O₃ (M+H)⁺: m/z=469.2; found: 469.2.

### Example 12: N-(5-chloro-3-phenylisoquinolin-6-yl)-5-((2-hydroxyethylamino)methyl) picolinamide

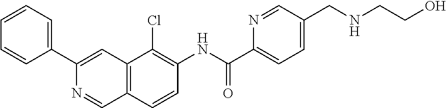 [SMILES: O=C(Nc1ccc2cnc(-c3ccccc3)cc2c1Cl)c1ccc(CNCCO)cn1]

### Step 1: methyl 5-((2-hydroxyethylamino)methyl)picolinate

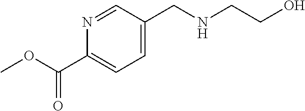

To a solution of methyl 5-formylpicolinate (AstaTech cat#68601: 525.0 mg, 3.179 mmol) in methanol (15 ml) was added ethanolamine (0.23 ml, 3.81 mmol). The reaction mixture was stirred at r.t. 30 min, then Pd/C 10% (50 mg) was added. The suspension was stirred at room temperature under an atmosphere of hydrogen for 1 hour. The suspension was filtered over silica gel and the filtrate was concentrated to dryness. The crude residue was purified by silica gel chromatography eluting with 15% MeOH in DCM to give desired product. LC-MS calculated for C₁₀H₁₅N₂O₃ (M+H)⁺: m/z=211.1; found: 211.1.

### Step 2: 3-phenylisoquinolin-6-amine

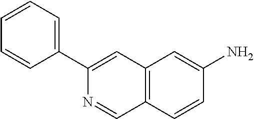 [SMILES: Nc1ccc2cnc(-c3ccccc3)cc2c1]

A mixture of Phenylboronic acid (Aldrich cat#P20009: 270 mg, 2.2 mmol), 3-chloroisoquinolin-6-amine (ArkPharm cat#AK476767: 200.0 mg, 1.120 mmol), sodium carbonate (300 mg, 2.8 mmol) and bis(di-cyclohexylphosphino)ferrocene]dichloropalladium(II) (85 mg, 0.11 mmol) in tert-butyl alcohol (7 mL) and water (7 mL) was degassed with nitrogen, then heated at 105° C. overnight. The crude reaction mixture was cooled to room temperature and filtered over celite and concentrated under reduced pressure. The crude residue was purified by silica gel chromatography eluting with 20% ethyl acetate in DCM to give the desired product. LC-MS calculated for C₁₅H₁₃N₂ (M+H)⁺: m/z=221.1; found: 221.1.

### Step 3: 5-chloro-3-phenylisoquinolin-6-amine

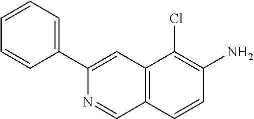 [SMILES: Nc1ccc2cnc(-c3ccccc3)cc2c1Cl]

To a solution of 3-phenylisoquinolin-6-amine (50.0 mg, 0.227 mmol) in acetonitrile (5 ml) was added N-chlorosuccinimide (33.3 mg, 0.250 mmol) and was stirred at room temperature for 4 hours. The crude reaction mixture was concentrated to dryness. The residue was dissolved in DCM and washed sequentially with water and brine. The organic layer was dried over Na₂SO₄, filtered, and concentrated under reduced pressure to give the desired product which was taken on without further purification. LC-MS calculated for C₁₅H₁₂ClN₂ (M+H)⁺: m/z=255.1; found: 255.1.

### Step 4: N-(5-chloro-3-phenylisoquinolin-6-yl)-5-((2-hydroxyethylamino)methyl)picolinamide

To the solution of 5-chloro-3-phenylisoquinolin-6-amine (70.0 mg, 0.275 mmol) in tetrahydrofuran (2 ml) was added 1.0M sodium hexamethyldisilazane in THF (0.316 mmol) at 0° C. and was allowed to stir for 30 min. To the stirring solution was then dropwise added ethyl 5-{[(2-hydroxyethyl)amino]methyl}pyridine-2-carboxylate (62 mg, 0.27 mmol) as a solution in THF (1 mL) and was stirred at room temperature for 2 hours. Water was added to quench the reaction and the crude reaction mixture was concentrated to dryness under reduced pressure. The residue was dissolved in MeOH, and was purified by prep-HPLC (pH=2, acetonitrile/water+TFA) to provide the desired compound as a white powder. LC-MS calculated for C₂₄H₂₂ClN₄O₂(M+H)⁺: m/z=433.1; found: 433.1.

### Example 13: 6-(2-methylbiphenyl-3-yl)-1,2,3,4-tetrahydroisoquinoline

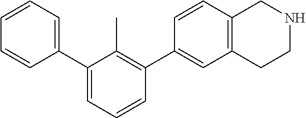 [SMILES: Cc1c(-c2ccccc2)cccc1-c1ccc2c(c1)CCNC2]

### Step 1: 6-(2-methylbiphenyl-3-yl)-3,4-dihydroisoquinolin-1 (2H)-one

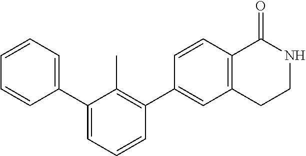 [SMILES: Cc1c(-c2ccccc2)cccc1-c1ccc2c(c1)CCNC2=O]

A mixture of 3-bromo-2-methylbiphenyl (Example 2, Step 1: 40 mg, 0.2 mmol), 6-(4,4,5,5-tetramethyl-1,3,2-dioxaborolan-2-yl)-3,4-dihydroisoquinolin-1 (2H)-one (Combi-Blocks cat# FM-2421: 53 mg, 0.19 mmol), [1,1′-bis(diphenylphosphino)ferrocene]dichloropalladium(II), complex with dichloromethane (1:1) (7 mg, 0.008 mmol) and potassium carbonate (67 mg, 0.48 mmol) in 1,4-dioxane (2 mL) and water (1 mL) was degassed and recharged with nitrogen three times. The mixture was then heated and stirred at 120° C. overnight. The reaction mixture was quenched with water, and extracted with ethyl acetate (3×50 mL). The combined organic layers were washed with brine, dried over MgSO₄, filtered and concentrated under reduced pressure to afford the crude product. LC-MS calculated for C₂₂H₂₀NO (M+H)⁺: m/z=314.2; found: 314.1.

### Step 2: 6-(2-methylbiphenyl-3-yl)-1,2,3,4-tetrahydroisoquinoline

1.0M Lithium tetrahydroaluminate in THF (0.48 mL) was added to a solution of the above product in THF (2 mL). The reaction mixture was stirred at 50° C. for 2 h. The reaction was cooled to room temperature and the reaction mixture was quenched with saturated aqueous NH₄Cl, and extracted with ethyl acetate (3×20 mL). The combined organic layers were washed with brine, dried over MgSO₄, filtered and concentrated under reduced pressure.

The residue was purified by prep LCMS (pH 2, acetonitrile/water+TFA) to give the desired product as its TFA salt. LC-MS calculated for C₂₂H₂₂N (M+H)⁺: m/z=300.2; found: 300.2.

### Example 14: 2-((2-(2-methylbiphenyl-3-yl)-1,2,3,4-tetrahydroisoquinolin-6-yl)methylamino)ethanol

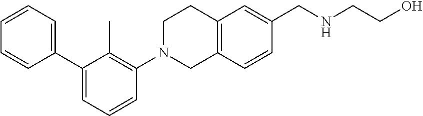 [SMILES: Cc1c(-c2ccccc2)cccc1N1CCc2cc(CNCCO)ccc2C1]

### Step 1: methyl 2-(2-methylbiphenyl-3-yl)-1,2,3,4-tetrahydroisoquinoline-6-carboxylate

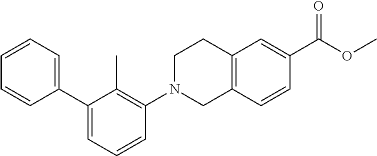 [SMILES: COC(=O)c1ccc2c(c1)CCN(c1cccc(-c3ccccc3)c1C)C2]

To a mixture of 3-bromo-2-methylbiphenyl (Example 2, Step 1: 270 mg, 1.09 mmol), palladium acetate (24 mg, 0.11 mmol), (R)-(+)-2,2′-bis(diphenylphosphino)-1,1′-binaphthyl (68 mg, 0.11 mmol), and cesium carbonate (1100 mg, 3.3 mmol) in 1,4-dioxane (10 mL) was added methyl 1,2,3,4-tetrahydroisoquinoline-6-carboxylate (AstaTech cat#F51533: 230 mg, 1.2 mmol) under N₂. The reaction mixture was stirred at 120° C. overnight. The crude reaction mixture was cooled to room temperature, was diluted with ethyl acetate, filtered, and concentrated under reduced pressure. The crude residue was purified by flash chromatography on a silica gel column with ethyl acetate in hexanes (0-30%) to afford the desired product. LC-MS calculated for C₂₄H₂₄NO₂ (M+H)⁺: m/z=358.2; found: 358.1.

### Step 2: (2-(2-methylbiphenyl-3-yl)-1,2,3,4-tetrahydroisoquinolin-6-yl)methanol

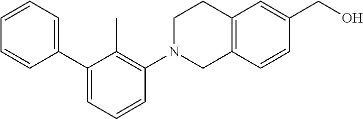 [SMILES: Cc1c(-c2ccccc2)cccc1N1CCc2cc(CO)ccc2C1]

1.0M Lithium tetrahydroaluminate in THF (2.2 mL) was added to a solution of the above product in THF (5 mL). The reaction mixture was stirred at room temperature for 2 h. The reaction mixture was quenched with saturated aqueous NH₄Cl, and extracted with ethyl acetate (3×20 mL). The combined organic layers were washed with brine, dried over MgSO₄, filtered and concentrated under reduced pressure. The residue was purified by flash chromatography on a silica gel column with ethyl acetate in hexanes (0-30%) to afford the desired product. LC-MS calculated for C₂₃H₂₄NO (M+H)⁺: m/z=330.2; found: 330.2.

### Step 3: (2-(2-methylbiphenyl-3-yl)-1,2,3,4-tetrahydroisoquinolin-6-yl)methyl methanesulfonate

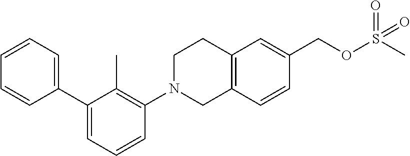 [SMILES: Cc1c(-c2ccccc2)cccc1N1CCc2cc(COS(C)(=O)=O)ccc2C1]

Methanesulfonyl chloride (0.32 mmol) was added to solution of [2-(2-methylbiphenyl-3-yl)-1,2,3,4-tetrahydroisoquinolin-6-yl]methanol (96 mg, 0.29 mmol) and N,N-diisopropylethylamine (0.44 mmol) in methylene chloride at 0° C. The reaction mixture was stirred at room temperature for 30 min, then quenched with saturated aqueous NaHCO₃, and extracted with ethyl acetate (3×20 mL). The combined organic layers were washed with brine, dried over MgSO₄, filtered and concentrated under reduced pressure to afford the desired product. LC-MS calculated for C₂₄H₂₆NO₃S (M+H)⁺: m/z=408.2; found: 408.1.

### Step 4: 2-((2-(2-methylbiphenyl-3-yl)-1,2,3,4-tetrahydroisoquinolin-6-yl)methylamino)ethanol

Ethanolamine (4.5 mg, 0.073 mmol) was added to solution of [2-(2-methylbiphenyl-3-yl)-1,2,3,4-tetrahydroisoquinolin-6-yl]methyl methanesulfonate (15 mg, 0.037 mmol) and N,N-diisopropylethylamine (0.1 mmol) in N,N-dimethylformamide. The reaction mixture was stirred at 60° C. for 6 h. The mixture was purified by prep LCMS (pH 2, acetonitrile/water+TFA) to give the desired product as its TFA salt. LC-MS calculated for C₂₅H₂₉N₂O (M+H)⁺: m/z 373.2; found: 373.2.

### Example 15: N-(2-methylbiphenyl-3-yl)-1,2,3,4-tetrahydroisoquinolin-6-amine

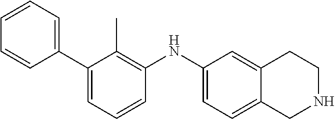 [SMILES: Cc1c(Nc2ccc3c(c2)CCNC3)cccc1-c1ccccc1]

### Step 1: tert-butyl 6-(2-methylbiphenyl-3-ylamino)-3,4-dihydroisoquinoline-2(1H)-carboxylate

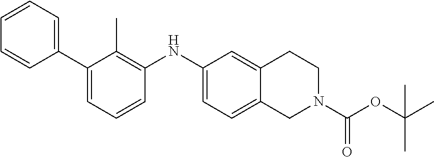 [SMILES: Cc1c(Nc2ccc3c(c2)CCN(C(=O)OC(C)(C)C)C3)cccc1-c1ccccc1]

To a mixture of 3-bromo-2-methylbiphenyl (Example 2, Step 1: 30 mg, 0.1 mmol), palladium acetate (2.7 mg, 0.012 mmol), (R)-(+)-2,2′-bis(diphenylphosphino)-1,1′-binaphthyl (7.6 mg, 0.012 mmol), and cesium carbonate (120 mg, 0.37 mmol) in 1,4-dioxane was added tert-butyl 6-amino-3,4-dihydroisoquinoline-2(1H)-carboxylate (Oakwood cat#011348: 33 mg, 0.13 mmol) under N₂. The reaction mixture was stirred at 120° C. overnight. The crude reaction mixture was cooled to room temperature, diluted with ethyl acetate, filtered and concentrated under reduced pressure. The residue was purified by flash chromatography on a silica gel column with ethyl acetate in hexanes (0-20%) to afford the desired product. LC-MS calculated for C₂₇H₃₁N₂O₂ (M+H)⁺: m/z 415.2; found: 415.2.

### Step 2: N-(2-methylbiphenyl-3-yl)-1,2,3,4-tetrahydroisoquinolin-6-amine

To a solution of the above compound in DCM (1 mL) was added TFA (1 mL). The reaction mixture was stirred at room temperature for 1 h. The crude reaction mixture was concentrated under reduced pressure. The isolated residue was purified by prep LCMS (pH 2, acetonitrile/water+TFA) to give the desired product as its TFA salt. LC-MS calculated for C₂₂H₂₃N₂(M+H)⁺: m/z 315.2; found: 315.2.

### Example 16: N,N-dimethyl-1-(6-(2-methylbiphenyl-3-yl)chroman-2-yl)methanamine

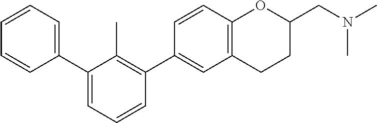 [SMILES: Cc1c(-c2ccccc2)cccc1-c1ccc2c(c1)CCC(CN(C)C)O2]

### Step 1: 6-bromo-N,N-dimethylchroman-2-carboxamide

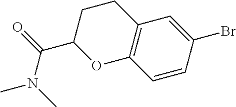 [SMILES: CN(C)C(=O)C1CCc2cc(Br)ccc2O1]

To a mixture of 6-bromochromane-2-carboxylic acid (Combi-Blocks cat#QB-8533: 0.38 g, 1.5 mmol) and 2.0M Dimethylamine in THF (2.2 mmol) in DMF (3 mL) was added Benzotriazol-1-yloxy)tripyrrolidinophosphonium hexafluorophosphate (940 mg, 1.8 mmol) followed by N,N-Diisopropylethylamine (2.2 mmol). The reaction mixture was stirred at room temperature for 2 hours. The crude reaction mixture was quenched with water and extracted with ethyl acetate. The combined organic layers were washed with brine, dried over magnesium sulfate, concentrated under reduced pressure, and used without further purification. LC-MS calculated for C₁₂H₁₅BrNO₂ (M+H)⁺: m/z 284.0; found: 284.0, 286.0.

### Step 2: N,N-dimethyl-6-(4,4,5,5-tetramethyl-1,3,2-dioxaborolan-2-yl)chromane-2-carboxamide

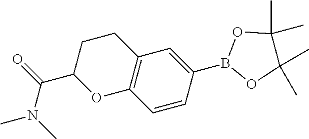

A mixture of 6-bromo-N,N-dimethylchromane-2-carboxamide (0.43 g, 1.5 mmol), 4,4,5,5,4′,4′,5′,5′-Octamethyl-[2,2′]bi[[1,3,2]dioxaborolanyl] (580 mg, 2.3 mmol), [1,1′-Bis(diphenylphosphino)ferrocene]dichloropalladium(II), complex with dichloromethane (1:1) (60 mg, 0.08 mmol), and potassium acetate (440 mg, 4.5 mmol) in 1,4-dioxane (200 mmol) was degassed and heated at 90° C. overnight. The crude reaction mixture was cooled to room temperature and concentrated under reduced pressure. The crude material was purified by flash column chromatography using 50% ethyl acetate in hexanes. LC-MS calculated for C₁₈H₂₇BNO₄ (M+H)⁺: m/z 332.2; found: 332.2.

### Step 3: N,N-dimethyl-6-(2-methylbiphenyl-3-yl)chroman-2-carboxamide

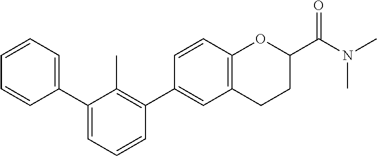 [SMILES: Cc1c(-c2ccccc2)cccc1-c1ccc2c(c1)CCC(C(=O)N(C)C)O2]

A mixture of 3-bromo-2-methylbiphenyl (Example 2, Step 1: 40 mg, 0.2 mmol), N,N-dimethyl-6-(4,4,5,5-tetramethyl-1,3,2-dioxaborolan-2-yl)chromane-2-carboxamide (64 mg, 0.19 mmol), [1,1′-bis(diphenylphosphino)ferrocene]dichloropalladium(II), complex with dichloromethane (1:1) (7 mg, 0.008 mmol) and potassium carbonate (67 mg, 0.48 mmol) in 1,4-dioxane (4 mL) and water (2 mL) was degassed and recharged with nitrogen three times. The mixture was then heated and stirred at 120° C. overnight. The reaction mixture was quenched with water, and extracted with ethyl acetate (3×50 mL). The combined organic layers were washed with brine, dried over MgSO₄, filtered and concentrated under reduced pressure to afford the crude product. LC-MS calculated for C₂₅H₂₆NO₂ (M+H)⁺: m/z 372.2; found: 372.2.

### Step 4: N,N-dimethyl-1-(6-(2-methylbiphenyl-3-yl)chroman-2-yl)methanamine

1.0M Lithium tetrahydroaluminate in THF (0.32 mL) was added to a solution of the above product in THF (2 mL). The reaction mixture was stirred at 50° C. for 2 h. The crude reaction mixture was cooled to room temperature and was quenched with saturated aqueous NH₄Cl, and extracted with ethyl acetate (3×20 mL). The combined organic layers were washed with brine, dried over MgSO₄, filtered and concentrated under reduced pressure. The residue was purified by prep LCMS (pH 2, acetonitrile/water+TFA) to give the desired product as its TFA salt. LC-MS calculated for C₂₅H₂₈NO (M+H)⁺: m/z 358.2; found: 358.2.

### Example 17: 3-(2-methylbiphenyl-3-yl)-6,7-dihydro-5H-pyrrolo[3,4-b]pyridine

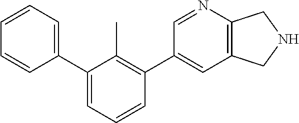 [SMILES: Cc1c(-c2ccccc2)cccc1-c1cnc2c(c1)CNC2]

A mixture of 4,4,5,5-tetramethyl-2-(2-methylbiphenyl-3-yl)-1,3,2-dioxaborolane (Example 4, Step 1: 50 mg, 0.2 mmol), 3-bromo-6,7-dihydro-5H-pyrrolo[3,4-b]pyridine (Synthonix cat#B11679: 37 mg, 0.19 mmol), [1,1′-bis(diphenylphosphino)ferrocene]dichloropalladium(II), complex with dichloromethane (1:1) (7 mg, 0.008 mmol) and potassium carbonate (70. mg, 0.51 mmol) in 1,4-dioxane (4 mL) and water (2 mL) was degassed and recharged with nitrogen three times. The mixture was then heated and stirred at 120° C. overnight. The reaction mixture was quenched with water, and extracted with ethyl acetate (3×50 mL). The combined organic layers were washed with brine, dried over MgSO₄, filtered and concentrated under reduced pressure to afford the crude product. The residue was purified by prep LCMS (pH 2, acetonitrile/water+TFA) to give the desired product as its TFA salt. LC-MS calculated for C₂₀H₁₉N₂(M+H)⁺: m/z 287.2; found: 287.2.

### Example 18: 2-(6-(2-methylbiphenyl-3-ylamino)-3,4-dihydroisoquinolin-2(1H)-yl)propane-1,3-diol

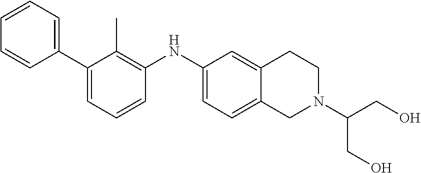 [SMILES: Cc1c(Nc2ccc3c(c2)CCN(C(CO)CO)C3)cccc1-c1ccccc1]

1,3-Dihydroxy-2-propanone (5.0 mg, 0.055 mmol) was added to a solution of N-(2-methylbiphenyl-3-yl)-1,2,3,4-tetrahydroisoquinolin-6-amine (TFA salt) (Example 15: 13 mg, 0.03 mmol) in N,N-dimethylformamide. The reaction mixture was stirred at room temperature for 10 min. Acetic acid (0.070 mmol) was added followed by sodium cyanoborohydride (8.9 mg, 0.14 mmol). The reaction mixture was allowed to stir overnight. The crude reaction mixture was purified by prep LCMS (pH 2, acetonitrile/water+TFA) to give the desired product as its TFA salt. LC-MS calculated for C₂₅H₂₉N₂O₂ (M+H)⁺: m/z 389.2; found: 389.2.

### Example 19: 2-((2-(2-methylbiphenyl-3-yl)isoindolin-5-yl)methylamino)ethanol

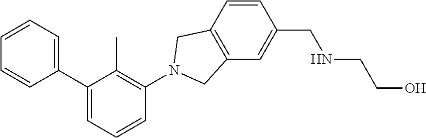 [SMILES: Cc1c(-c2ccccc2)cccc1N1Cc2ccc(CNCCO)cc2C1]

### Step 1: methyl 2-(2-methylbiphenyl-3-yl)isoindoline-5-carboxylate

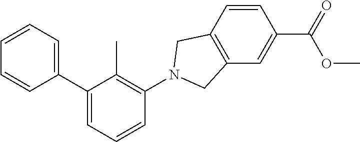 [SMILES: COC(=O)c1ccc2c(c1)CN(c1cccc(-c3ccccc3)c1C)C2]

To a mixture of 3-bromo-2-methylbiphenyl (Example 2, Step 1: 0.20 g, 0.81 mmol), Palladium Acetate (17 mg, 0.075 mmol), (R)-(+)-2,2′-bis(diphenylphosphino)-1,1′-binaphthyl (47 mg, 0.075 mmol), and cesium carbonate (0.73 g, 2.2 mmol) in 1,4-dioxane (1 mL) was added methyl isoindoline-5-carboxylate hydrochloride (AstaTech cat#63466: 0.16 g, 0.75 mmol) under N₂. The reaction mixture was stirred at 110° C. overnight. After the crude reaction mixture was cooled to room temperature the reaction mixture was diluted with ethyl acetate, filtered and concentrated under reduced pressure. The residue was purified by flash chromatography on a silica gel column with ethyl acetate in hexanes (0-10%) to afford the desired product. LC-MS calculated for C₂₃H₂₂NO₂ (M+H)⁺: m/z 344.2; found: 344.3.

### Step 2: (2-(2-methylbiphenyl-3-yl)isoindolin-5-yl)methanol

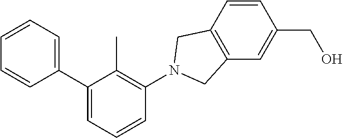 [SMILES: Cc1c(-c2ccccc2)cccc1N1Cc2ccc(CO)cc2C1]

To a solution of the above product in THF (4 mL) was added 1.0M Lithium tetrahydroaluminate in THF (1.1 mL). The reaction mixture was stirred at room temperature for 2 h. The crude reaction mixture was quenched with saturated aqueous NH₄Cl, and extracted with ethyl acetate (3×20 mL). The combined organic layers were washed with brine, dried over MgSO₄, filtered, and concentrated under reduced pressure to afford the desired product. LC-MS calculated for C₂₂H₂₂NO (M+H)⁺: m/z 316.2; found: 316.1.

### Step 3: (2-(2-methylbiphenyl-3-yl)isoindolin-5-yl)methyl methanesulfonate

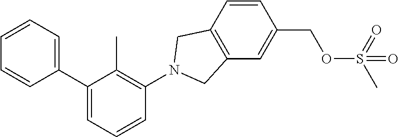 [SMILES: Cc1c(-c2ccccc2)cccc1N1Cc2ccc(COS(C)(=O)=O)cc2C1]

Methanesulfonyl chloride (0.48 mmol) was added to solution of [2-(2-methylbiphenyl-3-yl)-2,3-dihydro-1H-isoindol-5-yl]methanol (101 mg, 0.320 mmol) and N,N-diisopropylethylamine (0.48 mmol) in dethylene chloride at 0° C. The reaction mixture was stirred at room temperature for 30 min after which time the crude reaction mixture was quenched with saturated aqueous NaHCO₃, and extracted with ethyl acetate (3×20 mL). The combined organic layers were washed with brine, dried over MgSO₄, filtered and concentrated under reduced pressure to afford the desired product. LC-MS calculated for C₂₃H₂₄NO₃S (M+H)⁺: m/z 394.1; found: 394.2.

### Step 4: 2-((2-(2-methylbiphenyl-3-yl)isoindolin-5-yl)methylamino)ethanol

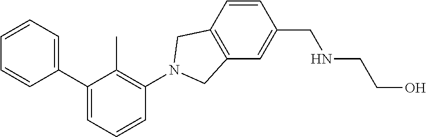 [SMILES: Cc1c(-c2ccccc2)cccc1N1Cc2ccc(CNCCO)cc2C1]

Ethanolamine (39 mg, 0.64 mmol) was added to a mixture of the above product and N,N-diisopropylethylamine (0.48 mmol) in DMF (2 mL). The reaction mixture was stirred at 60° C. for 2 h. The mixture was adjusted to pH2 with TFA, and purified by prep LCMS (pH 2, acetonitrile/water+TFA) to give the desired product as its TFA salt. LC-MS calculated for C₂₄H₂₇N₂O (M+H)⁺: m/z 359.2; found: 359.2.

### Example 20: 3-(2-methylbiphenyl-3-yl)-5,6,7,8-tetrahydro-1,6-naphthyridine

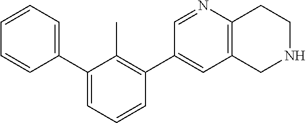 [SMILES: Cc1c(-c2ccccc2)cccc1-c1cnc2c(c1)CNCC2]

A mixture of 4,4,5,5-tetramethyl-2-(2-methylbiphenyl-3-yl)-1,3,2-dioxaborolane (Example 4, Step 1: 20 mg, 0.07 mmol), 3-bromo-5,6,7,8-tetrahydro-1,6-naphthyridine hydrochloride (AstaTech cat# SC2711: 17 mg, 0.068 mmol), [1,1′-bis(diphenylphosphino)ferrocene]dichloropalladium(II), complex with dichloromethane (1:1) (3 mg, 0.003 mmol) and potassium carbonate (28 mg, 0.20 mmol) in 1,4-dioxane (1 mL) and water (0.5 mL) was degassed and recharged with nitrogen three times. The mixture was then heated and stirred at 110° C. overnight. The crude reaction mixture was purified by prep LCMS (pH 2, acetonitrile/water+TFA) to give the desired product as its TFA salt. LC-MS calculated for C₂₁H₂₁N₂(M+H)⁺: m/z 301.2; found: 301.2.

### Example 21: (4-(3-methyl-4-phenylpyridin-2-yl)phenyl)methanamine

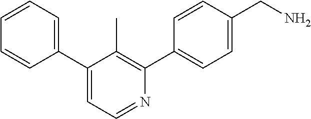 [SMILES: Cc1c(-c2ccccc2)ccnc1-c1ccc(CN)cc1]

### Step 1: 2-chloro-3-methyl-4-phenylpyridine

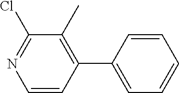

To a solution of 2-chloro-4-iodo-3-methylpyridine (Aldrich cat#724092: 303 mg, 1.20 mmol), phenylboronic acid (160 mg, 1.32 mmol), and sodium carbonate (317 mg, 2.99 mmol) in tert-butyl alcohol (9.5 mL) and Water (5.4 mL) was added bis(di-cyclohexylphosphino)ferrocene]dichloropalladium(II) (181 mg, 0.239 mmol). The reaction was purged with N2, then heated to 80° C. The crude reaction mixture was cooled to room temperature after 2 hours. The crude reaction mixture was diluted with water and extracted with DCM. The combined organic layers were washed with brine, dried over magnesium sulfate, and concentrated under reduced pressure. The crude residue was purified by column chromatography (0˜20% ethyl acetate in hexanes). LC-MS calculated for C₁₂H₁₁ClN (M+H)⁺: m/z 204.1; found: 204.2.

### Step 2: (4-(3-methyl-4-phenylpyridin-2-yl)phenyl)methanamine

A mixture of 2-chloro-3-methyl-4-phenylpyridine (20 mg, 0.1 mmol), [4-(aminomethyl)phenyl]boronic acid hydrochloride (Combi-Blocks cat# BB-2443: 22 mg, 0.12 mmol), [1,1′-bis(diphenylphosphino)ferrocene]dichloropalladium(II), complex with dichloromethane (1:1) (7 mg, 0.008 mmol) and potassium carbonate (70. mg, 0.51 mmol) in 1,4-dioxane (1 mL) and water (0.5 mL) was degassed and recharged with nitrogen three times. The mixture was then heated and stirred at 110° C. overnight. The reaction mixture was quenched with saturated aqueous NaHCO₃, and extracted with ethyl acetate (3×20 mL). The combined organic layers were washed with brine, dried over MgSO₄, filtered and concentrated under reduced pressure. The residue was purified by prep LCMS (pH 2, acetonitrile/water+TFA) to give the desired product as its TFA salt. LC-MS calculated for C₁₉H₁₉N₂(M+H)⁺: m/z 275.2; found: 275.2.

### Example 22: Cis-4-((5-(3-phenylpiperidin-1-yl)pyridin-2-yl)methylamino)cyclohexanol

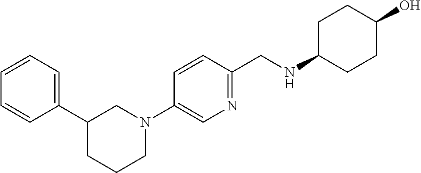 [SMILES: O[C@H]1CC[C@@H](NCc2ccc(N3CCCC(c4ccccc4)C3)cn2)CC1]

### Step 1: 5-(3-phenylpiperidin-1-yl)picolinaldehyde

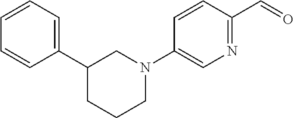 [SMILES: O=Cc1ccc(N2CCCC(c3ccccc3)C2)cn1]

To a mixture of 5-bromopyridine-2-carbaldehyde (Combi-Blocks cat#CA-4232: 0.16 g, 0.86 mmol), palladium acetate (18 mg, 0.080 mmol), (R)-(+)-2,2′-bis(diphenylphosphino)-1,1′-binaphthyl (50. mg, 0.080 mmol), and cesium carbonate (0.78 g, 2.4 mmol) in 1,4-dioxane (1 mL) was added 3-phenylpiperidine (Oakwood cat#019443: 0.13 g, 0.80 mmol) under N₂. The reaction mixture was stirred at 110° C. overnight. After the crude reaction mixture was cooled to room temperature it was diluted with ethyl acetate, filtered, and concentrated under reduced pressure to afford the crude product which was taken on without further purification. LC-MS calculated for C₁₇H₁₉N₂O (M+H)⁺: m/z 267.1; found: 267.1.

### Step 2: Cis-4-((5-(3-phenylpiperidin-1-yl)pyridin-2-yl)methylamino)cyclohexanol

Cis-4-Aminocyclohexanol hydrochloride (Aldrich cat#740365: 8.3 mg, 0.055 mmol) was added to a solution of 5-(3-phenylpiperidin-1-yl)pyridine-2-carbaldehyde (10 mg, 0.04 mmol) in N,N-dimethylformamide, followed by acetic acid (0.11 mmol). After 5 min, sodium cyanoborohydride (6.9 mg, 0.11 mmol) was added. The reaction mixture was stirred at room temperature overnight. The crude reaction mixture was purified by prep LCMS (pH 2, acetonitrile/water+TFA) to give the desired product as its TFA salt. LC-MS calculated for C₂₃H₃₂N₃O (M+H)⁺: m/z 366.3; found: 366.3.

### Example 23: (1-((1-(2-chloro-3-(phenylamino)phenyl)piperidin-4-ylamino)methyl)cyclobutyl)methanol

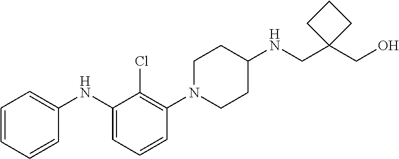 [SMILES: OCC1(CNC2CCN(c3cccc(Nc4ccccc4)c3Cl)CC2)CCC1]

### Step 1: 8-(3-bromo-2-chlorophenyl)-1,4-dioxa-8-azaspiro[4.5]decane

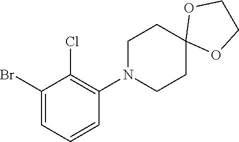 [SMILES: Clc1c(Br)cccc1N1CCC2(CC1)OCCO2]

To a mixture of 1,3-dibromo-2-chlorobenzene (Combi-Blocks cat#QA-2717: 2.2 g, 8.14 mmol), Pd(OAc)₂ (0.183 g, 0.814 mmol) and cesium carbonate (6.63 g, 20.34 mmol) in 1,4-dioxane (30 ml) was added 1,4-dioxa-8-azaspiro[4.5]decane (Aldrich cat#178365: 1.165 g, 8.14 mmol) under N₂. The reaction mixture was stirred at 90° C. overnight. After the reaction was cooled to room temperature it was quenched with saturated aqueous NaHCO₃, and extracted with ethyl acetate (3×50 mL). The combined organic layers were washed with brine, dried over MgSO₄, filtered and concentrated under reduced pressure. The residue was purified by flash chromatography on a silica gel column with ethyl acetate in hexanes (0-20%) to afford the desired product. LC-MS calculated for C₁₃H₁₆BrClNO₂ (M+H)⁺: m/z 332.0; found: 332.0/334.0.

### Step 2: 1-(3-bromo-2-chlorophenyl)piperidin-4-one

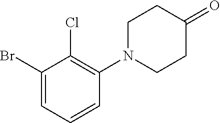 [SMILES: O=C1CCN(c2cccc(Br)c2Cl)CC1]

To a solution of the above product in acetone (4 mL) was added 1N HCl (4 mL) in water and MeOH (4 mL). The reaction mixture was stirred at 40° C. overnight. The reaction mixture was quenched with saturated aqueous NaHCO₃, and extracted with ethyl acetate (3×20 mL). The combined organic layers were washed with brine, dried over MgSO₄, filtered and concentrated under reduced pressure to afford the desired product. LC-MS calculated for C₁₁H₁₂BrClNO (M+H)⁺: m/z 288.0; found: 288.0/290.0.

### Step 3: (1-((1-(3-bromo-2-chlorophenyl)piperidin-4-ylamino)methyl)cyclobutyl)methanol

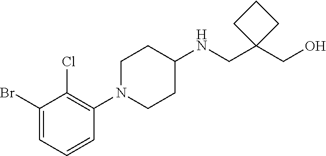 [SMILES: OCC1(CNC2CCN(c3cccc(Br)c3Cl)CC2)CCC1]

(1-(Aminomethyl)cyclobutyl)methanol (Combi-Blocks cat# AM-2310: 0.369 g, 3.20 mmol) was added to a solution of 1-(3-bromo-2-chlorophenyl)piperidin-4-one (0.77 g, 2.67 mmol) in N,N-dimethylformamide (10 ml), followed by acetic acid (0.611 ml, 10.67 mmol). After 5 min, sodium cyanoborohydride (0.335 g, 5.34 mmol) was added. The reaction mixture was stirred at room temperature overnight. The mixture was quenched with sat. NaHCO₃, extracted with ethyl acetate (3×20 mL), washed with brine, dried over MgSO₄, filtered and concentrated under reduced pressure. The residue was purified by flash chromatography on a silica gel column with MeOH in DCM (0-5%) to afford the desired product. LC-MS calculated for C₁₇H₂₅BrClN₂O (M+H)⁺: m/z 387.1; found: 387.0/389.1/391.0.

### Step 4: (1-((1-(2-chloro-3-(phenylamino)phenyl)piperidin-4-ylamino)methyl)cyclobutyl)methanol

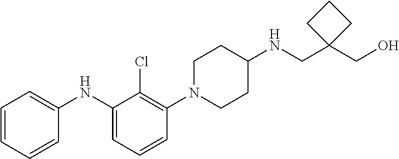 [SMILES: OCC1(CNC2CCN(c3cccc(Nc4ccccc4)c3Cl)CC2)CCC1]

To a mixture of (1-(((1-(3-bromo-2-chlorophenyl)piperidin-4-yl)amino)methyl)cyclobutyl)methanol (20 mg, 0.052 mmol), (R)-(+)-2,2′-bis(diphenylphosphino)-1,1′-binaphthyl (3.21 mg, 5.16 μmol), and cesium carbonate (42.0 mg, 0.129 mmol) in 1,4-dioxane (1 ml) was added aniline (7.21 mg, 0.077 mmol) under N₂. The reaction mixture was stirred at 120° C. overnight. After the reaction mixture was cooled to room temperature it was quenched with saturated aqueous NaHCO₃, and extracted with ethyl acetate (3×50 mL). The combined organic layers were washed with brine, dried over MgSO₄, filtered and concentrated under reduced pressure. The residue was purified by prep LCMS (pH 2, acetonitrile/water+TFA) to give the desired product as its TFA salt. LC-MS calculated for C₂₃H₃₁ClN₃O (M+H)⁺: m/z 400.2; found: 400.2.

### Example 24: (1-((1-(3-(benzylamino)-2-chlorophenyl)piperidin-4-ylamino)methyl)cyclobutyl)methanol

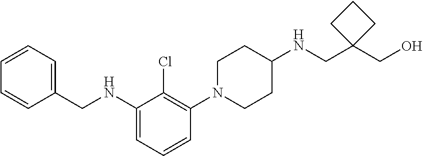 [SMILES: OCC1(CNC2CCN(c3cccc(NCc4ccccc4)c3Cl)CC2)CCC1]

To a mixture of (1-(((1-(3-bromo-2-chlorophenyl)piperidin-4-yl)amino)methyl)cyclobutyl)methanol (Example 23, Step 3: 20 mg, 0.052 mmol), (R)-(+)-2,2′-bis(diphenylphosphino)-1,1′-binaphthyl (3.21 mg, 5.16 μmol), and cesium carbonate (42.0 mg, 0.129 mmol) in 1,4-Dioxane (1 ml) was added phenylmethanamine (8.29 mg, 0.077 mmol) under N₂. The reaction mixture was stirred at 120° C. overnight. After the reaction mixture was cooled to room temperature it was quenched with saturated aqueous NaHCO₃, and extracted with ethyl acetate (3×50 mL). The combined organic layers were washed with brine, dried over MgSO₄, filtered and concentrated under reduced pressure. The residue was purified by prep LCMS (pH 2, acetonitrile/water+TFA) to give the desired product as its TFA salt. LC-MS calculated for C₂₄H₃₃ClN₃O (M+H)⁺: m/z 414; found: 414.2.

### Example 25: N-(2-methylbiphenyl-3-yl)isothiazolo[4,5-b]pyrazin-3-amine

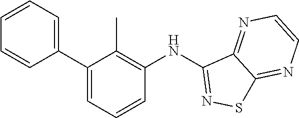 [SMILES: Cc1c(Nc2nsc3nccnc23)cccc1-c1ccccc1]

A mixture of 3-bromoisothiazolo[4,5-b]pyrazine (Ark Pharm, cat#AK-30773: 17 mg, 0.080 mmol), 2-methylbiphenyl-3-amine (Example 11, Step 1: 14.7 mg, 0.080 mmol), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (BrettPhos Pd G3, 11 mg, 0.012 mmol), and cesium carbonate (130 mg, 0.40 mmol) in tert-butyl alcohol was purged with nitrogen, and then stirred at 100° C. for 2 h. After being cooled to room temperature, the crude reaction mixture was diluted with methanol and purified by prep HPLC (pH=2, acetonitrile/water+TFA) to provide the desired compound as the TFA salt. LC-MS calculated for C₁₈H₁₅N₄S (M+H)+: m/z=319.1; found 319.2.

### Example 26: (R)-1-(2-methylbiphenyl-3-yl)piperidin-3-amine

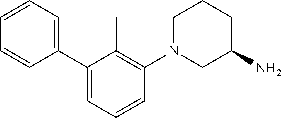 [SMILES: Cc1c(-c2ccccc2)cccc1N1CCC[C@@H](N)C1]

A stirred mixture of tert-butyl (3R)-piperidin-3-ylcarbamate (Combi-Blocks Cat#AM-1743: 0.016 g, 0.081 mmol), 3-bromo-2-methylbiphenyl (Example 2, Step 1: 10.0 mg, 0.0405 mmol), (2′-aminobiphenyl-2-yl)(chloro)[dicyclohexyl(2′,6′-diisopropoxybiphenyl-2-yl)phosphoranyl]palladium (3.09 mg, 0.00397 mmol), sodium tert-butoxide (7.64 mg, 0.0795 mmol) in 1,4-dioxane (2.0 mL) was heated at 110° C. under the atmosphere of N₂ overnight. The reaction was quenched with water, and extracted with ethyl acetate (3×10 mL). The combined organic layers were washed with brine, dried over Na₂SO₄, filtered and concentrated under reduced pressure. The residue was dissolved in DCM/TFA (0.5 mL/0.5 mL) and stirred at room temperature for 1 h. The volatiles were removed under reduced pressure and the crude product was purified on prep LCMS (pH 2, acetonitrile/water+TFA) to give the desired product as its TFA salt. LC-MS calculated for C₁₈H₂₃N₂(M+H)⁺: m/z=267.2; found 267.2.

TABLE 1
The compounds in Table 1 were prepared in accordance with the synthetic
protocols set forth in Example 26, using the appropriate starting materials.

| | | | LC-MS |
|---|---|---|---|
| Example | Name | Structure | (M + H)+ |
| 27 | (1-(2-methylbiphenyl-3- yl)piperidin-4-yl)methanamine | 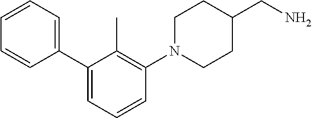 [SMILES: Cc1c(-c2ccccc2)cccc1N1CCC(CN)CC1] | 281.2 |
| 28 | 2-methyl-N-(piperidin-3- ylmethyl)biphenyl-3-amine | 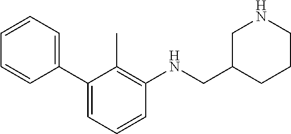 [SMILES: Cc1c(NCC2CCCNC2)cccc1-c1ccccc1] | 281.2 |
| 29 | (R)-(1-(2-methylbiphenyl-3- yl)pyrrolidin-3- yl)methanamine | 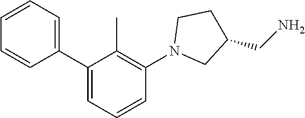 [SMILES: Cc1c(-c2ccccc2)cccc1N1CC[C@H](CN)C1] | 267.2 |
| 30 | 9-(2-methylbiphenyl-3-yl)-1,9- diazaspiro[5.5]undecan-2-one | 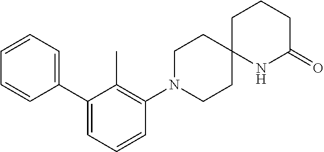 [SMILES: Cc1c(-c2ccccc2)cccc1N1CCC2(CCCC(=O)N2)CC1] | 335.2 |

### Example 31: 9-(2-methylbiphenyl-3-yl)-1,9-diazaspiro[5.5]undecane

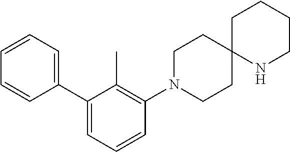 [SMILES: Cc1c(-c2ccccc2)cccc1N1CCC2(CCCCN2)CC1]

To a stirred solution of 9-(2-methylbiphenyl-3-yl)-1,9-diazaspiro[5.5]undecan-2-one (Example 30: 10.0 mg, 0.0299 mmol) in tetrahydrofuran (2 mL), 1.0M Lithium aluminum hydride in THF (0.18 mL, 0.18 mmol) was added at room temperature. The resulting mixture was stirred at room temperature overnight. The reaction was then quenched with saturated aqueous NH₄Cl and extracted with DCM (3×10 mL). The combined organic layers were dried over Na₂SO₄ and filtered. The filtrate was concentrated under reduced pressure and the residue was purified on prep LCMS (pH 2, acetonitrile/water+TFA) to give the desired product as its TFA salt. LC-MS calculated for C₂₂H₂₉N₂(M+H)⁺: m/z=321.2; found 321.2.

### Example 32: 1-(4-(2-methylbiphenyl-3-yl)cyclohex-3-enyl)pyrrolidine

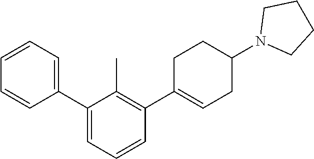 [SMILES: Cc1c(C2=CCC(N3CCCC3)CC2)cccc1-c1ccccc1]

A mixture of 1-[4-(4,4,5,5-tetramethyl-1,3,2-dioxaborolan-2-yl)cyclohex-3-en-1-yl]pyrrolidine (ArkPharm cat#AK141420: 0.033 g, 0.12 mmol), 3-bromo-2-methylbiphenyl (Example 2 Step 1: 0.019 g, 0.079 mmol), sodium carbonate (18.4 mg, 0.173 mmol), and bis(di-cyclohexylphosphino)ferrocene]dichloropalladium(II) (6.0 mg, 0.0079 mmol) in tert-butyl alcohol (1 mL)/water (1 mL) was first degassed with nitrogen, then stirred and heated at 100° C. for 2 hours. The crude reaction mixture was diluted with MeOH and purified on prep LCMS (pH 2, acetonitrile/water+TFA) to give the desired product as its TFA salt. LC-MS calculated for C₂₃H₂₈N (M+H)⁺: m/z=318.2; found 318.2.

### Example 33: 3-amino-N-(2-methylbiphenyl-3-yl)piperidine-1-carboxamide

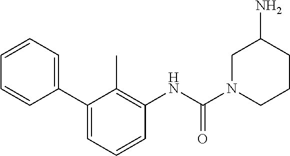 [SMILES: Cc1c(NC(=O)N2CCCC(N)C2)cccc1-c1ccccc1]

To a solution of triphosgene (8.1 mg, 0.027 mmol) in DCM (2 mL) at 0° C. was added pyridine (4.98 mg, 0.063 mmol). After 10 minutes, a solution of 2-methylbiphenyl-3-amine (Example 11, step 1: 10.0 mg, 0.0546 mmol) in DCM (3 mL) was added dropwise and was allowed to stir for 1 hour. To the stirring solution was then slowly added tert-butyl piperidin-3-ylcarbamate (Combi Blocks cat#AM-1743: 0.033 g, 0.16 mmol), followed by the addition of N,N-diisopropylethylamine (14.2 mg, 0.11 mmol). The resulting red suspension was warmed to room temperature and stirred for an additional 2 hours. The crude reaction mixture was then quenched with saturated aqueous NaHCO₃, and extracted with ethyl acetate (3×10 mL). The combined organic layers were washed with brine, dried over Na₂SO₄, filtered and the filtrate was concentrated under reduced pressure. The residue was dissolved in DCM/TFA (0.5 mL/0.5 mL) and stirred at room temperature for 1 hour. The volatiles were removed and the crude reaction mixture was purified on prep LCMS (pH 2, acetonitrile/water+TFA) to give the desired product as its TFA salt. LC-MS calculated for C₁₉H₂₄N₃O (M+H)⁺: m/z=310.2; found 310.2.

### Example 34: 2-[(3-{[(2-hydroxyethyl)amino]methyl}-1,7-naphthyridin-8-yl)amino]-4-phenylthiophene-3-carbonitrile

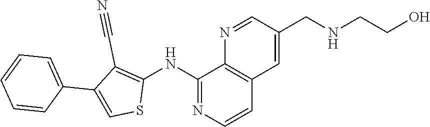 [SMILES: N#Cc1c(-c2ccccc2)csc1Nc1nccc2cc(CNCCO)cnc12]

### Step 1: 8-chloro-3-vinyl-1,7-naphthyridine

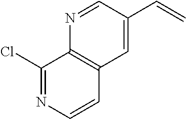

A mixture of 3-bromo-8-chloro-1,7-naphthyridine (PharmaBlock cat#PBLJ2743: 0.200 g, 0.821 mmol), 4,4,5,5-tetramethyl-2-vinyl-1,3,2-dioxaborolane (Aldrich cat#663348: 153 μL, 0.904 mmol), sodium carbonate (0.174 g, 1.64 mmol) and [1,1′-bis(dicyclohexylphosphino)ferrocene]dichloropalladium(II) (Aldrich cat#701998: 6.2 mg, 0.0082 mmol) in tert-butyl alcohol (5.91 mL, 61.8 mmol) and water (6 mL, 300 mmol) was degassed and sealed. It was stirred at 110° C. for 2 h. The reaction mixture was cooled to room temperature then extracted with ethyl acetate (3×20 mL). The combined organic layers were washed with brine, dried over MgSO₄, filtered and concentrated under reduced pressure. The crude residue was used directly in the next step without further purification. LC-MS calculated for C₁₀H₈ClN₂ (M+H)⁺: m/z=191.0; found 191.0.

### Step 2: 8-chloro-1,7-naphthyridine-3-carbaldehyde

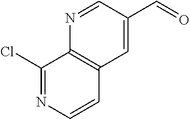 [SMILES: O=Cc1cnc2c(Cl)nccc2c1]

A flask was charged with 8-chloro-3-vinyl-1,7-naphthyridine (391. mg, 2.05 mmol), 1,4-dioxane (40. mL), a stir bar and water (40. mL). To this suspension was added a 4% w/w mixture of osmium tetroxide in water (0.84 mL, 0.132 mmol). The reaction was stirred for 5 min then sodium periodate (3.23 g, 15.11 mmol) was added and stirred for 3 h. The mixture was diluted with water (20 mL) and EtOAc (20 mL). The layers were separated and the aqueous layer was further extracted with EtOAc (2×20 mL). The combined organic extracts were washed with brine, dried over sodium sulfate, filtered, and concentrated under reduced pressure. The crude aldehyde was purified by silica gel chromatography (0→60% EtOAc/hexanes). LC-MS calculated for C₉H₆ClN₂O (M+H)⁺: m/z=193.0; found 192.9.

### Step 3: 2-{[(8-chloro-1,7-naphthyridin-3-yl)methyl]amino}ethanol

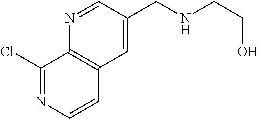 [SMILES: OCCNCc1cnc2c(Cl)nccc2c1]

A mixture of 8-chloro-1,7-naphthyridine-3-carbaldehyde (0.160 g, 0.831 mmol) and ethanolamine (Aldrich cat#398136: 251 μL, 4.15 mmol) in methylene chloride (6 mL, 100 mmol) and N,N-diisopropylethylamine (868 μL, 4.98 mmol) was stirred at room temperature for 1 h. Sodium triacetoxyborohydride (0.528 g, 2.49 mmol) was carefully added in portions. The reaction was stirred at room temperature for 2 h. To the mixture was then carefully added sodium tetrahydroborate (157 mg, 4.15 mmol) and methanol (1 mL) and the reaction mixture was stirred overnight under nitrogen. The reaction was quenched with a saturated aqueous solution of sodium bicarbonate. The mixture was then extracted with a 3:1 mixture of chlorofom/isopropyl alcohol. The combined organic layers were washed with brine, dried over sodium sulfate, and concentrated in vacuo. The crude residue was purified by column chromatography (0→50% methanol/DCM) and was obtained as an off white solid. LC-MS calculated for C₁₁H₁₃ClN₃O (M+H)⁺: m/z=238.1; found 238.1.

### Step 4: 2-[(3-{[(2-hydroxyethyl)amino]methyl}-1,7-naphthyridin-8-yl)amino]-4-phenylthiophene-3-carbonitrile

To a vial was added 2-amino-4-phenylthiophene-3-carbonitrile (Combi-Blocks, cat#QA-7728: 0.0168 g, 0.0841 mmol), 2-{[(8-chloro-1,7-naphthyridin-3-yl)methyl]amino}ethanol (10.00 mg, 0.04207 mmol), cesium carbonate (0.0274 g, 0.0841 mmol), 1,4-dioxane (12.8 mmol), (9,9-dimethyl-9H-xanthene-4,5-diyl)bis(diphenylphosphine) (4.9 mg, 0.0084 mmol), and tris(dibenzylideneacetone)dipalladium(0) (4.4 mg, 0.0042 mmol). A stir bar was added and the mixture was degassed, sealed, and heated for 2 h at 100° C. After cooling, the mixture was diluted with methanol and purified by prep HPLC (pH=2, water+TFA) to afford the desired compound as the TFA salt. LC-MS calculated for C₂₂H₂₀N₅OS (M+H)⁺: m/z=402.1; found 402.2.

### Example 35: (R)-2-(dimethylamino)-1-(2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazol-5(6H)-yl)ethanone

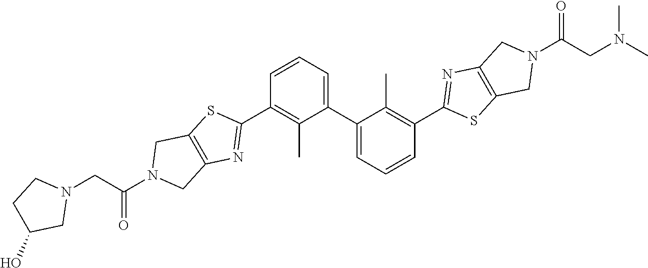 [SMILES: Cc1c(-c2nc3c(s2)CN(C(=O)CN(C)C)C3)cccc1-c1cccc(-c2nc3c(s2)CN(C(=O)CN2CC[C@@H](O)C2)C3)c1C]

### Step 1: tert-butyl 2-bromo-4H-pyrrolo[3,4-d]thiazole-5(6H)-carboxylate

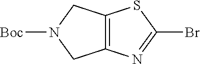 [SMILES: CC(C)(C)OC(=O)N1Cc2nc(Br)sc2C1]

To a stirred solution of 2-bromo-5,6-dihydro-4H-pyrrolo[3,4-d]thiazole, HBr (Aurum Pharm, cat# MR22320: 220.0 mg, 0.769 mmol) and N,N-diisopropylethylamine (0.269 ml, 1.539 mmol) in DCM (5.0 ml), was added Boc-anhydride (201 mg, 0.923 mmol) at room temperature. After 1 hour, the reaction mixture was diluted with EtOAc (100 mL), and washed with water (3×15 mL). The organic layer was dried over Na₂SO₄, filtered and the filtrate was concentrated under reduced pressure to afford the title compound (220 mg, 0.724 mmol, 93.6% yield), which was used directly in the next step without further purification. LC-MS calculated for C₁₀H₁₄BrN₂O₂S (M+H)⁺: m/z=305.0/307.0; found 305.0/307.0.

### Step 2: tert-butyl 2-(3-chloro-2-methylphenyl)-4H-pyrrolo[3,4-d]thiazole-5(6H)-carboxylate

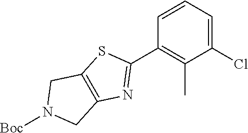 [SMILES: Cc1c(Cl)cccc1-c1nc2c(s1)CN(C(=O)OC(C)(C)C)C2]

A mixture of (3-chloro-2-methylphenyl)boronic acid (Combi Blocks cat#BB-2035: 335 mg, 1.966 mmol), tert-butyl 2-bromo-4,6-dihydro-5H-pyrrolo[3,4-d]thiazole-5-carboxylate (600.0 mg, 1.966 mmol), tetrakis(triphenylphosphine)palladium(0) (114 mg, 0.098 mmol) and sodium carbonate (521 mg, 4.91 mmol) in dioxane (8 mL) and water (2 mL) was degassed and sealed. It was stirred at 100° C. overnight. After the reaction mixture was cooled to room temperature, it was diluted with EtOAc (100 mL), and washed with water. The organic layer was dried over Na₂SO₄, filtered and the filtrate was concentrated under reduced pressure. The residue was purified by chromatography on silica gel, eluting with 0-40% EtOAc/hexanes, to give the desired product (590 mg). LC-MS calculated for C₁₇H₂₀ClN₂O₂S (M+H)⁺: m/z=351.1; found 351.1.

### Step 3: tert-butyl 2-(2-methyl-3-(4,4,5,5-tetramethyl-1,3,2-dioxaborolan-2-yl)phenyl)-4H-pyrrolo[3,4-d]thiazole-5(6H)-carboxylate

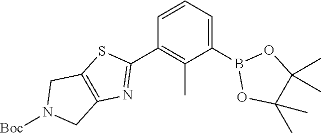 [SMILES: Cc1c(B2OC(C)(C)C(C)(C)O2)cccc1-c1nc2c(s1)CN(C(=O)OC(C)(C)C)C2]

A mixture of tert-butyl 2-(3-chloro-2-methylphenyl)-4,6-dihydro-H-pyrrolo[3,4-d]thiazole-5-carboxylate (268 mg, 0.764 mmol), 4,4,4′,4′,5,5,5′,5′-octamethyl-2,2′-bi(1,3,2-dioxaborolane) (291 mg, 1.146 mmol), tris(dibenzylideneacetone)dipalladium(0) (56.0 mg, 0.061 mmol), 2-dicyclohexylphosphino-2′,4′,6′-tri-iso-propyl-1,1′-biphenyl (58.3 mg, 0.122 mmol) and potassium acetate (150 mg, 1.528 mmol) in 1,4-dioxane (8 mL) was degassed with N₂ and was stirred at 100° C. for 2.5 h. After cooling to room temperature, the reaction mixture was diluted with DCM and filtered. The filtrate was concentrated under reduced pressure and the crude product was purified by chromatography on silica gel, eluting with 0-30% EtOAc/hexanes, to give the desired product (269 mg). LC-MS calculated for C₂₃H₃₂BN₂O₄S (M+H)⁺: m/z=443.2; found 443.2.

### Step 4: tert-butyl 2-(3′-bromo-2,2′-dimethylbiphenyl-3-yl)-4H-pyrrolo[3,4-d]thiazole-5(6H)-carboxylate

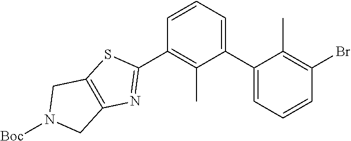 [SMILES: Cc1c(Br)cccc1-c1cccc(-c2nc3c(s2)CN(C(=O)OC(C)(C)C)C3)c1C]

A mixture of 1,3-dibromo-2-methylbenzene (Combi Blocks cat#OT-1437: 339 mg, 1.356 mmol), tert-butyl 2-(2-methyl-3-(4,4,5,5-tetramethyl-1,3,2-dioxaborolan-2-yl)phenyl)-4,6-dihydro-5H-pyrrolo[3,4-d]thiazole-5-carboxylate (200.0 mg, 0.452 mmol), sodium carbonate (96 mg, 0.904 mmol) and tetrakis(triphenylphosphine)palladium(0) (52.2 mg, 0.045 mmol) in dioxane (3.00 mL)/water (1.0 mL) was heated at 90° C. overnight. The reaction was then cooled to room temperature, diluted with saturated aqueous NH₄Cl, and extracted with EtOAc (3×30 mL). The combined organic layers were washed with brine, dried over Na₂SO₄, filtered, and the filtrate was concentrated under reduced pressure. The crude residue was purified by chromatography on silica gel, eluting with 0-40% EtOAc/hexanes, to give the desired product (210 mg). LC-MS calculated for C₂₄H₂₆BrN₂O₂S (M+H)⁺: m/z=485.1/487.1; found 485.0/487.0.

### Step 5: 1-(2-(3′-bromo-2,2′-dimethylbiphenyl-3-yl)-4H-pyrrolo[3,4-d]thiazol-5(6H)-yl)-2-chloroethanone

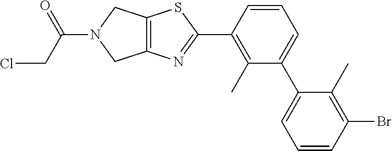 [SMILES: Cc1c(Br)cccc1-c1cccc(-c2nc3c(s2)CN(C(=O)CCl)C3)c1C]

Tert-butyl 2-(3′-bromo-2,2′-dimethylbiphenyl-3-yl)-4H-pyrrolo[3,4-d]thiazole-5 (6H)-carboxylate (210 mg, 0.433 mmol) was dissolved in TFA/DCM (1 mL/1 mL) at room temperature. After 1 h, the volatiles were removed under reduced pressure and the residue was dissolved in dichloromethane (3.0 mL). Hunig's base (0.237 mL, 1.356 mmol) and 2-chloroacetyl chloride (56.2 mg, 0.497 mmol) were added sequentially at room temperature and the resulting mixture was stirred for 30 min. The reaction was then quenched with saturated aqueous NH₄Cl, and extracted with EtOAc (3×30 mL). The combined organic layers were washed with brine, dried over Na₂SO₄, filtered and the filtrate was concentrated under reduced pressure. The crude residue was purified by chromatography on silica gel, eluting with 0-40% ethyl acetate/hexanes, to give the desired product (181 mg). LC-MS calculated for C₂₁H₁₉BrClN₂OS (M+H)⁺: m/z=461.0/463.0; found 460.9/462.9.

### Step 6: (R)-1-(2-(3′-bromo-2,2′-dimethylbiphenyl-3-yl)-4H-pyrrolo[3,4-d]thiazol-5(6H)-yl)-2-(3-hydroxypyrrolidin-1-yl)ethanone

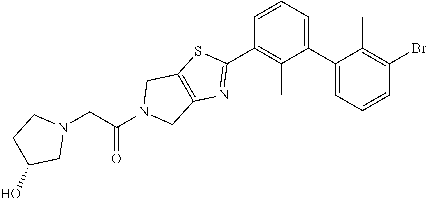 [SMILES: Cc1c(Br)cccc1-c1cccc(-c2nc3c(s2)CN(C(=O)CN2CC[C@@H](O)C2)C3)c1C]

To a stirred solution of 1-(2-(3′-bromo-2,2′-dimethyl-[1,1′-biphenyl]-3-yl)-4,6-dihydro-5H-pyrrolo[3,4-d]thiazol-5-yl)-2-chloroethan-1-one (50.0 mg, 0.108 mmol) and (R)-pyrrolidin-3-ol (11.32 mg, 0.130 mmol) in acetonitrile (2.0 ml), Hunig's base (0.038 ml, 0.217 mmol) was added at room temperature. The resulting mixture was stirred at 60° C. for 2 hours. The volatiles were removed under reduced pressure and the residue was purified by chromatography on silica gel, eluting with 0-15% MeOH/DCM, to give the desired product (45 mg). LC-MS calculated for C₂₅H₂₇BrN₃O₂S (M+H)⁺: m/z=512.1/514.1; found 512.1/514.1.

### Step 7: (R)-tert-butyl 2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazole-5 (6H)-carboxylate

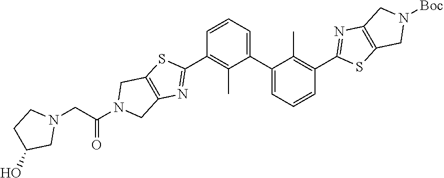 [SMILES: Cc1c(-c2nc3c(s2)CN(C(=O)CN2CC[C@@H](O)C2)C3)cccc1-c1cccc(-c2nc3c(s2)CN(C(=O)OC(C)(C)C)C3)c1C]

A mixture of (R)-1-(2-(3′-bromo-2,2′-dimethyl-[1,1′-biphenyl]-3-yl)-4,6-dihydro-5H-pyrrolo[3,4-d]thiazol-5-yl)-2-(3-hydroxypyrrolidin-1-yl)ethan-1-one (120 mg, 0.234 mmol), Bis(pinacolato)diboron (71.4 mg, 0.281 mmol), dichloro[1,1′-bis(diphenylphosphino)ferrocene]palladium (II) dichloromethane adduct (19.12 mg, 0.023 mmol) and potassium acetate (46.0 mg, 0.468 mmol) in dioxane (5 mL) was charged with nitrogen and stirred at 110° C. for 2 h. After the reaction mixture was cooled to room temperature, tert-butyl 2-bromo-4,6-dihydro-5H-pyrrolo[3,4-d]thiazole-5-carboxylate (71.5 mg, 0.234 mmol), another portion of dichloro[1,1′-bis(diphenylphosphino)ferrocene]palladium (II) dichloromethane adduct (19.12 mg, 0.023 mmol), sodium carbonate (49.6 mg, 0.468 mmol), and water (1 mL) were added sequentially. The resulting mixture was heated at 110° C. for 3 h. After the mixture was cooled to room temperature, the reaction quenched with saturated aqueous NaHCO₃, and extracted with ethyl acetate (3×10 mL). The combined organic layers were washed with brine, dried over Na₂SO₄, filtered and the filtrate was concentrated under reduced pressure. The residue was purified on prep LCMS (pH 2, acetonitrile/water+TFA) to give the desired product (52 mg) as its TFA salt. LC-MS calculated for C₃₅H₄₀N₅O₄S₂(M+H)⁺: m/z=658.3; found 658.2.

### Step 8: (R)-1-(2-(3′-(5,6-dihydro-4H-pyrrolo[3,4-d]thiazol-2-yl)-2,2′-dimethylbiphenyl-3-yl)-4H-pyrrolo[3,4-d]thiazol-5(6H)-yl)-2-(3-hydroxypyrrolidin-1-yl)ethanone

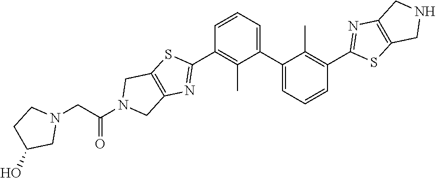 [SMILES: Cc1c(-c2nc3c(s2)CNC3)cccc1-c1cccc(-c2nc3c(s2)CN(C(=O)CN2CC[C@@H](O)C2)C3)c1C]

(R)-tert-butyl 2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazole-5(6H)-carboxylate (52 mg, 0.079 mmol) was dissolved in TFA (1 mL)/DCM (1 mL) and stirred at room temperature. After 1 h, the volatiles were removed under reduced pressure to afford the crude product, which was used directly in the next step without further purification. LC-MS calculated for C₃₀H₃₂N₅O₂S₂(M+H)⁺: m/z=558.2; found 558.2.

### Step 9: (R)-2-(dimethylamino)-1-(2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazol-5(6H)-yl)ethanone

To a stirred solution of (R)-1-(2-(3′-(5,6-dihydro-4H-pyrrolo[3,4-d]thiazol-2-yl)-2,2′-dimethyl-[1,1′-biphenyl]-3-yl)-4,6-dihydro-5H-pyrrolo[3,4-d]thiazol-5-yl)-2-(3-hydroxypyrrolidin-1-yl)ethan-1-one (10.0 mg, 0.018 mmol) and dimethylglycine (3.70 mg, 0.036 mmol) in DMF (1.0 ml), N,N,N′,N′-tetramethyl-O-(7-azabenzotriazol-1-yl)uronium hexafluorophosphate (6.82 mg, 0.018 mmol), and N,N-diisopropylethylamine (3.75 μl, 0.022 mmol) were added sequentially at room temperature. After 1 hour, the mixture was diluted with acetonitrile and purified by prep LCMS (pH 2, acetonitrile/water+TFA) to give the desired product as its TFA salt. LC-MS calculated for C₃₄H₃₉N₆O₃S₂(M+H)⁺: m/z=643.3; found 643.2.

### Example 36: 1-(2-(2,2′-dimethyl-3′-(piperidin-4-ylethynyl)biphenyl-3-yl)-4H-pyrrolo[3,4-d]thiazol-5(6H)-yl)-2-(ethyl(methyl)amino)ethanone

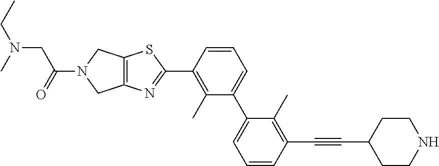 [SMILES: CCN(C)CC(=O)N1Cc2nc(-c3cccc(-c4cccc(C#CC5CCNCC5)c4C)c3C)sc2C1]

### Step 1: 2-chloro-1-(2-(3-chloro-2-methylphenyl)-4H-pyrrolo[3,4-d]thiazol-5(6H)-yl)ethanone

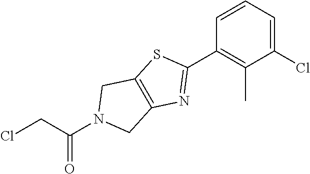 [SMILES: Cc1c(Cl)cccc1-c1nc2c(s1)CN(C(=O)CCl)C2]

To a stirred solution of tert-butyl 2-(3-chloro-2-methylphenyl)-4,6-dihydro-5H-pyrrolo[3,4-d]thiazole-5-carboxylate (Example 35, step 2: 408 mg, 1.163 mmol) in DCM (2 mL) at room temperature, TFA (2 mL) was added. After 1 h, the volatiles were removed and the residue was dissolved in DCM (3 mL). Hunig's base (0.406 mL, 2.326 mmol) and 2-chloroacetyl chloride (0.102 mL, 1.279 mmol) were then added sequentially at room temperature. After and additional hour, the reaction mixture was quenched with saturated aq. NaHCO₃, extracted with DCM (3×50 mL). The organic layers were combined, dried over Na₂SO₄, filtered and the filtrate was concentrated under reduced pressure. The residue was purified by chromatography on silica gel, eluting with 0-30% EtOAc/hexanes, to give the desired product (362 mg). LC-MS calculated for C₁₄H₁₃Cl₂N₂OS (M+H)⁺: m/z=327.0; found 327.0.

### Step 2: 1-(2-(3-chloro-2-methylphenyl)-4H-pyrrolo[3,4-d]thiazol-5(6H)-yl)-2-(ethyl(methyl)amino)ethanone

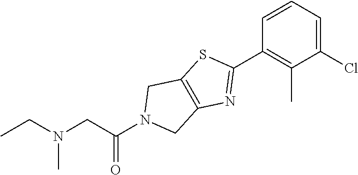 [SMILES: CCN(C)CC(=O)N1Cc2nc(-c3cccc(Cl)c3C)sc2C1]

To a stirred solution of 2-chloro-1-(2-(3-chloro-2-methylphenyl)-4,6-dihydro-5H-pyrrolo[3,4-d]thiazol-5-yl)ethan-1-one (200.0 mg, 0.611 mmol) in acetonitrile (3.0 ml), N-methylethanamine (36.1 mg, 0.611 mmol) and Hunig's base (107 μl, 0.611 mmol) were added at room temperature. The resulting mixture was heated at 60° C. After 2 h, the reaction mixture was quenched with saturated aq. NaHCO₃, and extracted with DCM (3×50 mL). The organic layers were combined, dried over Na₂SO₄, filtered and the filtrate was concentrated under reduced pressure. The residue was purified by chromatography on silica gel, eluting with 0-5% MeOH/DCM, to give the desired product (203 mg). LC-MS calculated for C₁₇H₂₁ClN₃OS (M+H)⁺: m/z=350.1; found 350.1.

### Step 3: 2-(ethyl(methyl)amino)-1-(2-(2-methyl-3-(4,4,5,5-tetramethyl-1,3,2-dioxaborolan-2-yl)phenyl)-4H-pyrrolo[3,4-d]thiazol-5(6H)-yl)ethanone

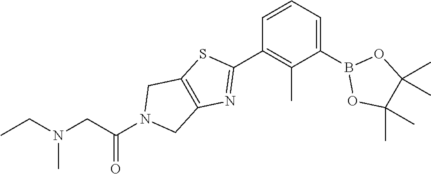 [SMILES: CCN(C)CC(=O)N1Cc2nc(-c3cccc(B4OC(C)(C)C(C)(C)O4)c3C)sc2C1]

A mixture of 1-(2-(3-chloro-2-methylphenyl)-4H-pyrrolo[3,4-d]thiazol-5(6H)-yl)-2-(ethyl(methyl)amino)ethanone (192 mg, 0.550 mmol), 4,4,4′,4′,5,5,5′,5′-octamethyl-2,2′-bi(1,3,2-dioxaborolane) (209 mg, 0.824 mmol), tris(dibenzylideneacetone)dipalladium(0) (40.3 mg, 0.044 mmol), 2-dicyclohexylphosphino-2′,4′,6′-tri-iso-propyl-1,1′-biphenyl (41.9 mg, 0.088 mmol) and potassium acetate (108 mg, 1.099 mmol) in 1,4-dioxane (5.0 ml) was degassed with N₂ and was stirred at 100° C. for 3 h. The reaction mixture was quenched with saturated aq. NH₄Cl, and extracted with DCM (3×50 mL). The organic layers were combined, dried over Na₂SO₄, filtered and the filtrate was concentrated under reduced pressure. The residue was purified by chromatography on silica gel, eluting with 0-10% MeOH/DCM, to give the desired product (168 mg). LC-MS calculated for C₂₃H₃₃BN₃O₃S (M+H)⁺: m/z=442.2; found 442.2.

### Step 4: tert-butyl 4-((3-bromo-2-methylphenyl)ethynyl)piperidine-1-carboxylate

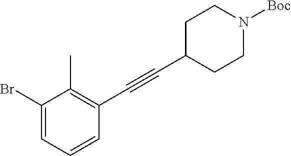 [SMILES: Cc1c(Br)cccc1C#CC1CCN(C(=O)OC(C)(C)C)CC1]

A mixture of 1,3-dibromo-2-methylbenzene (Combi-Blocks cat#OT-1437: 143 mg, 0.573 mmol), tert-butyl 4-ethynylpiperidine-1-carboxylate (ArkPharm catalog # AK-34528: 60 mg, 0.287 mmol), copper(I) iodide (4.37 mg, 0.023 mmol), dichlorobis(triphenylphosphine)-palladium(II) (26.8 mg, 0.038 mmol), and triethylamine (0.080 ml, 0.573 mmol) in 1,4-Dioxane (3.0 ml) was flushed with N₂. The resulting slurry was stirred at 90° C. for 3 h. The reaction was then quenched with water, extracted with EtOAc (3×15 mL). The organic layers were combined, dried over Na₂SO₄, filtered, and the filtrate was concentrated under reduced pressure. The crude residue was purified by chromatography on silica gel, eluting with 0-35% ethyl acetate/hexanes, to give the desired product. LC-MS calculated for C₁₅H₁₇BrNO₂ (M+H-tBu)⁺: m/z=322.0; found 322.0.

### Step 5: 1-(2-(2,2′-dimethyl-3′-(piperidin-4-ylethynyl) biphenyl-3-yl)-4H-pyrrolo[3,4-d]thiazol-5(6H)-yl)-2-(ethyl(methyl)amino)ethanone

A mixture of tert-butyl 4-((3-bromo-2-methylphenyl)ethynyl)piperidine-1-carboxylate (8.57 mg, 0.023 mmol), 2-(ethyl(methyl)amino)-1-(2-(2-methyl-3-(4,4,5,5-tetramethyl-1,3,2-dioxaborolan-2-yl)phenyl)-4,6-dihydro-5H-pyrrolo[3,4-d]thiazol-5-yl)ethan-1-one (10.0 mg, 0.023 mmol), sodium carbonate (4.80 mg, 0.045 mmol) and bis(di-cyclohexylphosphino)ferrocene]dichloropalladium(II) (1.717 mg, 2.266 μmol) in t-BuOH (0.800 mL)/water (0.8 mL) was heated at 90° C. for 2 h. The reaction was then quenched with water, and extracted with EtOAc (3×10 mL). The organic layers were combined, dried over Na₂SO₄, filtered, and the filtrate was concentrated under reduced pressure. The residue was purified by chromatography on silica gel, eluting with 0-10% MeOH/DCM, to give the coupling product. The purified coupling product was dissolved in TFA (0.5 mL)/DCM (0.5 mL) and stirred at room temperature for 1 hour after which time the volatiles were removed and the crude residue was purified on prep LCMS (pH 2, acetonitrile/water+TFA) to give the desired product as its TFA salt. LC-MS calculated for C₃₁H₃₇N₄OS (M+H)⁺: m/z=513.3; found 513.3.

### Example 37: 1-(2-(2,2′-dimethyl-3′-(tetrahydro-1H-pyrrolo[3,2-c]pyridin-5(6H,7H,7aH)-yl)biphenyl-3-yl)-4H-pyrrolo[3,4-d]thiazol-5(6H)-yl)-2-(ethyl(methyl)amino)ethanone

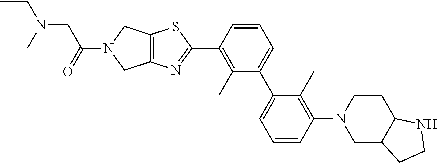 [SMILES: CCN(C)CC(=O)N1Cc2nc(-c3cccc(-c4cccc(N5CCC6NCCC6C5)c4C)c3C)sc2C1]

### Step 1: tert-butyl 5-(3-bromo-2-methylphenyl)octahydro-1H-pyrrolo[3,2-c]pyridine-1-carboxylate

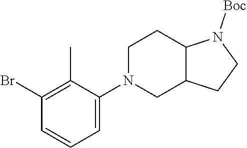 [SMILES: Cc1c(Br)cccc1N1CCC2C(CCN2C(=O)OC(C)(C)C)C1]

A mixture of tert-butyl octahydro-1H-pyrrolo[3,2-c]pyridine-1-carboxylate (Combi-Blocks catalog # ST-7254: 60 mg, 0.265 mmol), 1,3-dibromo-2-methylbenzene (Combi-Blocks cat# OT-1437: 199 mg, 0.795 mmol), palladium(II) acetate (5.95 mg, 0.027 mmol), (R)-(+)-2,2′-bis(diphenylphosphino)-1,1′-binaphthyl (16.51 mg, 0.027 mmol), and cesium carbonate (173 mg, 0.530 mmol) in 1,4-Dioxane (5.0 ml) was flushed with N₂. The resulting slurry was stirred at 90° C. overnight. After being cooled to room temperature, the reaction mixture was quenched with saturated aqueous NaHCO₃, and extracted with ethyl acetate (3×10 mL). The combined organic layers were washed with brine, dried over Na₂SO₄, filtered and concentrated under reduced pressure. The residue was purified by flash chromatography on a silica gel, eluting with ethyl acetate in hexanes (0-50%) to afford the desired product. LC-MS calculated for C₁₉H₂₈BrN₂O₂ (M+H)⁺: m/z=395.1; found 395.1.

### Step 2: 1-(2-(2,2′-dimethyl-3′-(tetrahydro-1H-pyrrolo[3,2-c]pyridin-5(6H, 7H, 7aH)-yl)biphenyl-3-yl)-4H-pyrrolo[3,4-d]thiazol-5(6H)-yl)-2-(ethyl(methyl)amino)ethanone

This compound was prepared using similar procedures as described for Example 36, Step 5 with tert-butyl 5-(3-bromo-2-methylphenyl)octahydro-1H-pyrrolo[3,2-c]pyridine-1-carboxylate replacing tert-butyl 4-((3-bromo-2-methylphenyl)ethynyl)piperidine-1-carboxylate. LC-MS calculated for C₃₁H₄₀N₅OS (M+H)⁺: m/z=530.3; found 530.4.

### Example 38: 1-(2-(3′-(5,6-dihydroimidazo[1,5-a]pyrazin-7(8H)-yl)-2,2′-dimethylbiphenyl-3-yl)-4H-pyrrolo[3,4-d]thiazol-5(6H)-yl)-2-(ethyl(methyl)amino)ethanone

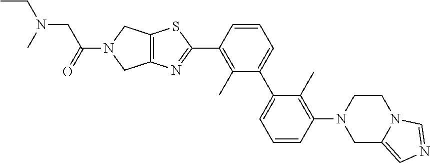 [SMILES: CCN(C)CC(=O)N1Cc2nc(-c3cccc(-c4cccc(N5CCn6cncc6C5)c4C)c3C)sc2C1]

This compound was prepared using similar procedures as described for Example 37 with 5,6,7,8-tetrahydroimidazo[1,5-a]pyrazine (Combi-Blocks, catalog # QB-0196) replacing tert-butyl octahydro-1H-pyrrolo[3,2-c]pyridine-1-carboxylate in Step 1 and without the treatment with TFA/DCM in Step 2. LC-MS calculated for C₃₀H₃₅N₆OS (M+H)⁺: m/z=527.3; found 527.2.

### Example 39: 2-(2′-fluoro-3′-methoxy-2-methylbiphenyl-3-ylamino)-N-(2-(2-hydroxyethylamino)ethyl)nicotinamide

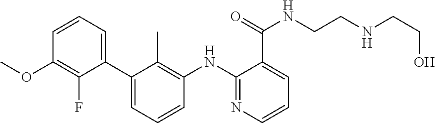 [SMILES: COc1cccc(-c2cccc(Nc3ncccc3C(=O)NCCNCCO)c2C)c1F]

### Step 1: 2′-fluoro-3′-methoxy-2-methylbiphenyl-3-amine

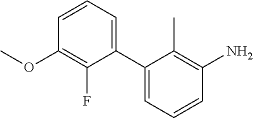 [SMILES: COc1cccc(-c2cccc(N)c2C)c1F]

To a vial was added 3-bromo-2-methylaniline (Aldrich, cat#530018: 0.331 ml, 2.69 mmol), (2-fluoro-3-methoxyphenyl)boronic acid (Aldrich, cat#594253: 0.502 g, 2.96 mmol), sodium carbonate (0.570 g, 5.37 mmol), 1,1′-bis(di-cyclohexylphosphino)ferrocene palladium dichloride (0.041 g, 0.054 mmol), 1,4-dioxane (12.5 mL) and water (4.5 mL). The mixture was degassed, sealed, and heated to 110° C. whilst stirring for 2 h. After cooling, the layers were separated and the aqueous layer was extracted with ethyl acetate (3×10 mL). The combined organic layers were washed with brine, dried over magnesium sulfate, filtered, and concentrated in vacuo. The crude residue was purified by silica gel chromatography (0→20% EtOAc/hexanes). LC-MS calculated for C₁₄H₁₅FNO (M+H)⁺: m/z=232.1; found 232.1.

### Step 2: ethyl 2-(2′-fluoro-3′-methoxy-2-methylbiphenyl-3-ylamino)nicotinate

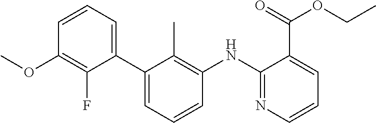 [SMILES: CCOC(=O)c1cccnc1Nc1cccc(-c2cccc(OC)c2F)c1C]

To a vial was added ethyl 2-chloronicotinate (Alfa Aesar, cat#B20359: 0.193 g, 1.038 mmol), 2′-fluoro-3′-methoxy-2-methyl-[1,1′-biphenyl]-3-amine (0.2 g, 0.865 mmol), 1,4-dioxane (7.21 ml), cesium carbonate (0.564 g, 1.730 mmol), 9,9-dimethyl-4,5-bis(diphenylphosphino)xanthene (0.060 g, 0.104 mmol), and tris(dibenzylideneacetone)dipalladium(0) (0.079 g, 0.086 mmol). The mixture was degassed and heated to 100° C. for 2 h. After cooling, the mixture was diluted with EtOAc and filtered through Celite®. The filtrate was concentrated under reduced pressure and purified by silica gel chromatography (50% EtOAc/hexanes) to provide the desired compound as an orange oil. LC-MS calculated for C₂₂H₂₂FN₂O₃(M+H)⁺: m/z=381.2; found 381.3.

### Step 3: 2-(2′-fluoro-3′-methoxy-2-methylbiphenyl-3-ylamino)nicotinic acid

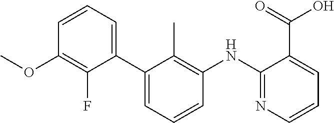 [SMILES: COc1cccc(-c2cccc(Nc3ncccc3C(=O)O)c2C)c1F]

To a vial was added ethyl 2-((2′-fluoro-3′-methoxy-2-methyl-[1,1′-biphenyl]-3-yl)amino)nicotinate (0.329 g, 0.865 mmol), MeOH (2.162 ml), THF (2.162 ml), and 2M LiOH in water (2.162 ml, 4.32 mmol). The mixture was stirred at rt for 1 h. The mixture was acidified using 2M citric acid, and the aqueous layer was extracted with 3:1 CHCl₃/IPA. The combined organic layers were dried over MgSO₄, filtered, and concentrated in vacuo. The crude acid was used directly in the next step. LC-MS calculated for C₂₀H₁₈FN₂O₃(M+H)⁺: m/z=353.1; found 353.2.

### Step 4: N-(2,2-dimethoxyethyl)-2-(2′-fluoro-3′-methoxy-2-methylbiphenyl-3-ylamino)nicotinamide

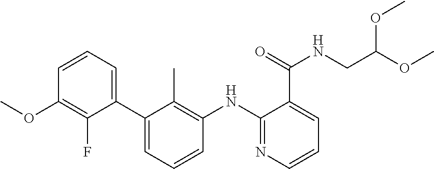

To a solution of 2-((2′-fluoro-3′-methoxy-2-methyl-[1,1′-biphenyl]-3-yl)amino)nicotinic acid (0.305 g, 0.866 mmol) in dichloromethane (4.33 mL) was added aminoacetaldehyde dimethyl acetal (Alfa Aesar, cat#A15498: 0.283 mL, 2.60 mmol), DIPEA (0.756 mL, 4.33 mmol), and HATU (0.987 g, 2.60 mmol) at rt. The resulting mixture was stirred at rt for 1 h. The mixture was diluted with water and DCM, and the layers were separated. The aqueous layer was further extracted with DCM, and the combined organic layers were dried over MgSO₄, filtered, and concentrated in vacuo to provide the desired product as a red oil which was used directly in the next step. LC-MS calculated for C₂₄H₂₇FN₃O₄(M+H)⁺: m/z=440.2; found 440.2.

### Step 5: 2-(2′-fluoro-3′-methoxy-2-methylbiphenyl-3-ylamino)-N-(2-oxoethyl)nicotinamide

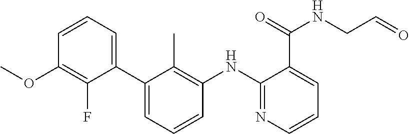 [SMILES: COc1cccc(-c2cccc(Nc3ncccc3C(=O)NCC=O)c2C)c1F]

To a solution of N-(2,2-dimethoxyethyl)-2-((2′-fluoro-3′-methoxy-2-methyl-[1,1′-biphenyl]-3-yl)amino)nicotinamide (0.392 g, 0.892 mmol) in DCM (8.92 ml) was added TFA (3.44 ml, 44.6 mmol) dropwise at rt. The mixture was stirred for 1 h, and was subsequently concentrated under reduced pressure. The resulting residue was re-dissolved in DCM, and washed with aqueous saturated NaHCO₃. The layers were separated and the organic phase was dried over MgSO₄, filtered, and concentrated in vacuo. The resulting crude oil was used directly in the next step without further purification. LC-MS calculated for C₂₂H₂₁FN₃O₃ (M+H)⁺: m/z=394.2; found 394.2.

### Step 6: 2-(2′-fluoro-3′-methoxy-2-methylbiphenyl-3-ylamino)-N-(2-(2-hydroxyethylamino)ethyl)nicotinamide

To a vial was added 2-((2′-fluoro-3′-methoxy-2-methyl-[1,1′-biphenyl]-3-yl)amino)-N-(2-oxoethyl)nicotinamide (0.020 g, 0.051 mmol), ethanolamine (Aldrich, cat#398136: 3.07 μL, 0.051 mmol), a stir bar, and DCE (0.508 mL). The mixture was stirred for 5 min, then sodium cyanoborohydride (9.58 mg, 0.153 mmol) and acetic acid (8.73 μL, 0.153 mmol) were added, and the mixture was stirred for 2 h at rt. The mixture was diluted with methanol and purified by prep HPLC (pH=2, acetonitrile/water+TFA) to afford the desired product as the TFA salt. LC-MS calculated for C₂₄H₂₈FN₄O₃(M+H)⁺: m/z=439.2; found 439.2.

### Example 40: 2-({3-chloro-4-[(4-phenyl-2,3-dihydro-1H-indol-1-yl)methyl]benzyl}amino)ethanol

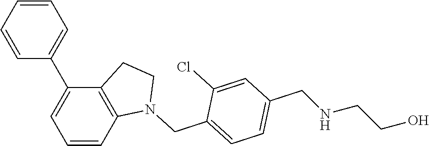 [SMILES: OCCNCc1ccc(CN2CCc3c(-c4ccccc4)cccc32)c(Cl)c1]

### Step 1: 4-phenylindoline

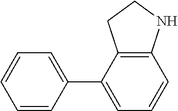 [SMILES: c1ccc(-c2cccc3c2CCN3)cc1]

A mixture of 4-bromoindoline (AstaTech, cat#BL008582: 253 mg, 1.28 mmol), phenylboronic acid (187 mg, 1.53 mmol), dichloro[1,1′-bis(dicyclohexylphosphino)ferrocene] palladium(II) (50 mg, 0.06 mmol) and potassium phosphate (540 mg, 2.6 mmol) in water (838 μl) and dioxane (4192 μl) was purged with N₂ and then stirred at 90° C. for 4 h. After cooling to room temperature, the mixture was concentrated and the corresponding residue was purified by column chromatography. LC-MS calculated for C₁₄H₁₄N (M+H)⁺: m/z=196.1; found 196.1.

### Step 2: methyl 3-chloro-4-[(4-phenyl-2,3-dihydro-1H-indol-1-yl)methyl]benzoate

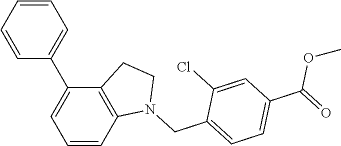 [SMILES: COC(=O)c1ccc(CN2CCc3c(-c4ccccc4)cccc32)c(Cl)c1]

To a solution of methyl 3-chloro-4-formylbenzoate (AstaTech, cat#CL9164: 123 mg, 0.619 mmol) and 4-phenylindoline (145 mg, 0.742 mmol) in DCM (5 ml) was added sodium triacetoxyborohydride (459 mg, 2.16 mmol) and acetic acid (0.62 mmol). The reaction was stirred at r.t. for 2 h. The mixture was then diluted with aqueous ammonium hydroxide, and was extracted with DCM three times. The combined DCM solutions were washed with water, brine and dried over MgSO₄. The DCM solution was then filtered and concentrated. The residue was purified by flash chromatography eluting with 0-15% EtOAc in hexanes. LC-MS calculated for C₂₃H₂₁ClNO₂ (M+H)⁺: m/z=378.1; found 378.1.

### Step 3: 3-chloro-4-[(4-phenyl-2,3-dihydro-1H-indol-1-yl)methyl]benzaldehyde

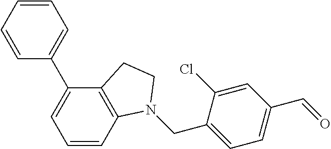 [SMILES: O=Cc1ccc(CN2CCc3c(-c4ccccc4)cccc32)c(Cl)c1]

To a solution of methyl 3-chloro-4-[(4-phenyl-2,3-dihydro-1H-indol-1-yl)methyl]benzoate (150 mg, 0.40 mmol) in THF (2 ml) was added diisobutylaluminum hydride (1.0M in THF, 0.95 mmol) slowly at 0° C. The mixture was slowly warmed up to r.t. and stirred for 1 h. The reaction was then quenched by EtOAc and Rochelle's salt solution. The mixture was vigorously stirred for 30 min. The organic layer was separated, concentrated and used directly for oxidation. LC-MS calculated for C₂₂H₂₁ClNO (M+H)⁺: m/z=350.1; found 350.1.

To the solution of above residue in DCM (2 ml) was added sodium bicarbonate (100 mg, 1.00 mmol) and Dess-Martin periodinane (220 mg, 0.52 mmol). The mixture was stirred at r.t. for 30 min. The reaction was quenched by aq. NaHCO₃ solution and aq. Na₂S₂O₃ solution. The mixture was extracted with DCM three times. The organic phase was combined, concentrated and purified by flash chromatography. LC-MS calculated for C₂₂H₁₉ClNO (M+H)⁺: m/z=348.1; found 348.1.

### Step 4: 3-chloro-4-[(4-phenyl-2,3-dihydro-1H-indol-1-yl)methyl]benzaldehyde

To a solution of 3-chloro-4-[(4-phenyl-2,3-dihydro-1H-indol-1-yl)methyl]benzaldehyde (30 mg, 0.09 mmol) and ethanolamine (0.11 mmol) in DCM (0.5 ml) was added sodium triacetoxyborohydride (27 mg, 0.13 mmol). After addition, the reaction was stirred at r.t. for 45 min. The reaction was then diluted in MeOH and purified by prep-HPLC (pH=10, acetonitrile/water+NH₄OH) to give the desired product. LC-MS calculated for C₂₄H₂₆ClN₂O (M+H)⁺: m/z=393.2; found 393.1.

### Example A. PD-1/PD-L1 Homogeneous Time-Resolved Fluorescence (HTRF) Binding Assay

The assays were conducted in a standard black 384-well polystyrene plate with a final volume of 20 μL. Inhibitors were first serially diluted in DMSO and then added to the plate wells before the addition of other reaction components. The final concentration of DMSO in the assay was 1%. The assays were carried out at 250° C. in the PBS buffer (pH 7.4) with 0.05% Tween-20 and 0.1% BSA. Recombinant human PD-L1 protein (19-238) with a His-tag at the C-terminus was purchased from AcroBiosystems (PD1-H5229). Recombinant human PD-1 protein (25-167) with Fc tag at the C-terminus was also purchased from AcroBiosystems (PD1-H5257). PD-L1 and PD-1 proteins were diluted in the assay buffer and 10 μL was added to the plate well. Plates were centrifuged and proteins were preincubated with inhibitors for 40 minutes. The incubation was followed by the addition of 10 μL of HTRF detection buffer supplemented with Europium cryptate-labeled anti-human IgG (PerkinElmer-AD0212) specific for Fc and anti-His antibody conjugated to SureLight®-Allophycocyanin (APC, PerkinElmer-AD0059H). After centrifugation, the plate was incubated at 250° C. for 60 min. before reading on a PHERAstar FS plate reader (665 nm/620 nm ratio). Final concentrations in the assay were—3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration using the GraphPad Prism 5.0 software.

Compounds of the present disclosure, as exemplified in the Examples, showed IC₅₀ values in the following ranges: +=IC₅₀≤10 nM; ++=10 nM<IC₅₀≤100 nM; +++=100 nM<IC₅₀≤1000 nM; ++++=1000 nM<IC50≤2000 nM Data obtained for the Example compounds using the PD-1/PD-L1 homogenous time-resolved fluorescence (HTRF) binding assay described in Example A is provided in Table 1A.

TABLE 1A

| | PD-1/PD-L1 HTRF |
|---|---|
| Example | IC₅₀ (nM) |
| 1 | ++ |
| 2 | ++ |
| 3 | + |
| 4 | ++ |
| 5 | ++ |
| 6 | ++ |
| 7 | ++ |
| 8 | ++ |
| 9 | ++ |
| 10 | +++ |
| 11 | +++ |
| 12 | +++ |
| 13 | ++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | ++++ |
| 24 | +++ |
| 25 | +++ |
| 26 | +++ |
| 27 | +++ |
| 28 | ++++ |
| 29 | ++++ |
| 30 | +++ |
| 31 | +++ |
| 32 | +++ |
| 33 | ++++ |
| 34 | +++ |
| 35 | + |
| 36 | + |
| 37 | +++ |
| 38 | ++ |
| 39 | +++ |
| 40 | +++ |

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A compound of Formula (I′): 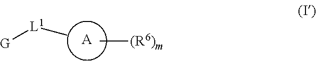
or a pharmaceutically acceptable salt or a stereoisomer thereof, wherein:
G has Formula (I′e) or (I′b) 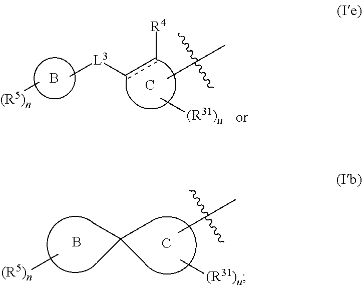
when G is of Formula (I′a), the atoms on ring C, to which the substituent R⁴ and ring B are attached can be either carbon or nitrogen; and [symbol] is a single bond or a double bond;
when G is of Formula (I′b), ring B and ring C are joined together through a quaternary ring carbon atom to form a spiro structure and ring B and ring C are each independently 4- to 14-membered heterocycloalkyl or C₃₋₁₄ cycloalkyl;
L¹ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, —(CR¹⁴R¹⁵)ₜC(═S)NR¹³(CR¹⁴R¹⁵)ₜ—, —(CR¹⁴R¹⁵)ₜNR¹³C(═S)(CR¹⁴R¹⁵)ₜ—, —(CR¹⁴R¹⁵)ₜC(═NR¹³)NR¹³(CR¹⁴R¹⁵)ₜ—, —(CR¹⁴R¹⁵)ₜNR¹³C(═NR¹³)(CR¹⁴R¹⁵)ₜ—, —(CR¹⁴R¹⁵)ₜC(═NOR¹³)NR¹³(CR¹⁴R¹⁵)ₜ—, —(CR¹⁴R¹⁵)ₜNR¹³C(═NOR¹³)(CR¹⁴R¹⁵)ₜ—, —(CR¹⁴R¹⁵)ₜC(═NCN)NR¹³(CR¹⁴R¹⁵)ₜ—, —(CR¹⁴R¹⁵)ₜNR¹³C(═NCN)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, S, —(CR¹⁴R¹⁵)ₚ—S—, —S(CR¹⁴R¹⁵)ₚ—, —(CR¹⁴R¹⁵)ₚ—S—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³SO₂NR¹³(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;
L³ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, —(CR¹⁴R¹⁵)ₜC(═S)NR¹³(CR¹⁴R¹⁵)ₜ—, —(CR¹⁴R¹⁵)ₜNR¹³C(═S)(CR¹⁴R¹⁵)ₜ—, —(CR¹⁴R¹⁵)ₜC(═NR¹³)NR¹³(CR¹⁴R¹⁵)ₜ—, —(CR¹⁴R¹⁵)ₜNR¹³C(═NR¹³)(CR¹⁴R¹⁵)ₜ—, —(CR¹⁴R¹⁵)ₜC(═NOR¹³)NR¹³(CR¹⁴R¹⁵)ₜ—, —(CR¹⁴R¹⁵)ₜNR¹³C(═NOR¹³)(CR¹⁴R¹⁵)ₜ—, —(CR¹⁴R¹⁵)ₜC(═NCN)NR¹³(CR¹⁴R¹⁵)ₜ—, —(CR¹⁴R¹⁵)ₜNR¹³C(═NCN)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, S, —(CR¹⁴R¹⁵)ₚ—S—, —S(CR¹⁴R¹⁵)ₚ—, —(CR¹⁴R¹⁵)ₚ—S—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³SO₂NR¹³(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;
ring A is C₆₋₁₀ aryl, 5- to 14-membered heteroaryl, 4- to 14-membered heterocycloalkyl or C₃₋₁₄ cycloalkyl;
ring B is C₆₋₁₀ aryl, 5- to 14-membered heteroaryl, 4- to 14-membered heterocycloalkyl or C₃₋₁₄ cycloalkyl;
ring C is C₆₋₁₀ aryl, 5- to 14-membered heteroaryl, 4- to 14-membered heterocycloalkyl or C₃₋₁₄ cycloalkyl;
each R¹³ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;
R¹⁴ and R¹⁵ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R¹⁴ or R¹⁵ are each optionally substituted with 1, 2, or 3 independently selected R^{q} substituents;
or R¹⁴ and R¹⁵ taken together with the carbon atom to which they are attached form C₃₋₆ cycloalkyl or 4- to 7-membered heterocycloalkyl, each of which is optionally substituted with 1, 2, or 3 independently selected R^{q} substituents;
R⁴ is H, halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl;
R⁵, R⁶ and R³¹ are each independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, C(O)NR^{a}S(O)₂R^{a}, NR^{a}C(═NR^{a})R^{a}, S(O)₂NR^{a}C(O)R^{a}, —P(O)R^{a}R^{a}, —P(O)(OR^{a})(OR^{a}), —B(OH)₂, —B(OR^{a})₂, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R⁵, R⁶ and R³¹ are each optionally substituted with 1, 2, 3, 4 or 5 independently selected R^{b} substituents;
or two adjacent R⁵ substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, B, P, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R⁵ substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, B, P, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
or two adjacent R⁶ substituents on ring A, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, B, P, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R⁶ substituents on the same ring carbon atom of the ring A, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, B, P, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
or two adjacent R³¹ substituents on ring C, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, B, P, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R³¹ substituents on the same ring carbon atom of ring C, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, B, P, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆-10 ariyl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2 or 3 independently selected R^{d} substituents;
each R^{b} substituent is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c}, C(O)NR^{c}S(O)₂R^{c}, NR^{c}C(═NR^{c})R^{c}, S(O)₂NR^{c}C(O)R^{c}, —P(O)R^{c}R^{c}, —P(O)(OR^{c})(OR^{c}), —B(OH)₂, —B(OR^{c})₂, and S(O)₂NR^{c}R^{c}; wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1, 2, or 3 independently selected R^{d} substituents;
or two R^{b} substituents attached to the same ring carbon atom taken together with the ring carbon atom to which they are attached form spiro C₃₋₆ cycloalkyl or spiro 4- to 7-membered heterocycloalkyl, each of which is optionally substituted with 1, 2, or 3 independently selected R^{f} substituents;
each R^{c} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2 or 3 independently selected R^{f} substituents;
each R^{f} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, C(O)NR^{g}S(O)₂R^{g}, NR^{g}C(═NR^{g})R^{g}, S(O)₂NR^{g}C(O)R^{g}, —P(O)R^{g}R^{g}, —P(O)(OR^{g})(OR^{g}), —B(OH)₂, —B(OR^{g})₂, and S(O)₂NR^{g}R^{g}; wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2 or 3 independently selected Rⁿ substituents;
each Rⁿ is independently selected from C₁₋₆ alkyl, C₁₋₆ haloalkyl, halo, CN, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, C(O)NR^{o}S(O)₂R^{o}, NR^{o}C(═NR^{o})R^{o}, S(O)₂NR^{o}C(O)R^{o}, —P(O)R^{o}R^{o}, —P(O)(OR^{o})(OR^{o}), —B(OH)₂, —B(OR^{o})₂, and S(O)₂NR^{o}R^{o}, wherein the C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of Rⁿ is optionally substituted with 1, 2 or 3 independently selected R^{q} substituents;
each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, C(O)NR^{e}S(O)₂R^{e}, NR^{e}C(═NR^{e})R^{e}, S(O)₂NR^{e}C(O)R^{e}, —P(O)R^{e}R^{e}, —P(O)(OR^{e})(OR^{e}), —B(OH)₂, —B(OR^{e})₂, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1, 2, or 3 independently selected R^{f} substituents;
each R^{e} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{e} are each optionally substituted with 1, 2 or 3 independently selected R^{f} substituents;
each R^{g} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1, 2, or 3 independently selected R^{p} substituents;
each R^{p} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r}, C(O)NR^{r}S(O)₂R^{r}, NR^{r}C(═NR^{r})R^{r}, S(O)₂NR^{r}C(O)R^{r}, —P(O)R^{r}R^{r}, —P(O)(OR^{r})(OR^{r}), —B(OH)₂, —B(OR^{r})₂, and S(O)₂NR^{r}R^{r}, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} is optionally substituted with 1, 2 or 3 independently selected R^{q} substituents;
or any two R^{a} substituents together with the nitrogen atom to which they are attached form a4-, 5-, 6-, 7-, 8-, 9- or 10-membered heterocycloalkyl group optionally substituted with 1, 2 or 3 independently selected R^{h} substituents;
each R^{h} is independently selected from C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, C₆₋₁₀ aryl-C₁₋₄ alkyl-, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, NRⁱC(═NOH)NRⁱRⁱ, NRⁱC(═NCN)NRⁱRⁱ, S(O)Rⁱ, S(O)NRⁱRⁱ, S(O)₂Rⁱ, NRⁱS(O)₂Rⁱ, NRⁱS(O)₂NRⁱRⁱ, C(O)NRⁱS(O)₂Rⁱ, NRⁱC(═NRⁱ)Rⁱ, S(O)₂NRⁱC(O)Rⁱ, P(O)RⁱRⁱ, —P(O)(ORⁱ)(ORⁱ), —B(OH)₂, —B(ORⁱ)₂, and S(O)₂NRⁱRⁱ, wherein the C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{h} are each optionally substituted by 1, 2, or 3 independently selected R^{j} substituents;
each R^{j} is independently selected from C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₂₋₄ alkynyl, halo, C₁₋₄ haloalkyl, C₁₋₄ haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR^{k})NR^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, C(O)NR^{k}S(O)₂R^{k}, NR^{k}C(═NR^{k})R^{k}, S(O)₂NR^{k}C(O)R^{k}, P(O)R^{k}R^{k}, —P(O)(OR^{k})(OR^{k}), —B(OH)₂, —B(OR^{k})₂, and S(O)₂NR^{k}R^{k}, wherein the C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₁₋₄ haloalkyl, and C₁₋₄ haloalkoxy of R^{j} are each optionally substituted with 1, 2 or 3 independently selected R^{q} substituents;
or two R^{h} groups attached to the same carbon atom of the 4- to 10-membered heterocycloalkyl, taken together with the carbon atom to which they are attached, form a C₃₋₆ cycloalkyl or 4- to 6-membered heterocycloalkyl having 1-2 heteroatoms as ring members selected from O, N or S;
or any two R^{c} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two R^{e} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two R^{g} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two Rⁱ substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents, or 1, 2, or 3 independently selected R^{q} substituents;
or any two R^{k} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents, or 1, 2, or 3 independently selected R^{q} substituents;
or any two R^{o} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents; and
or any two R^{r} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
each Rⁱ, R^{k}, R^{o} or R^{r} is independently selected from H, C₁₋₆ alkyl, C₁₋₆ haloalkyl, C₃₋₆ cycloalkyl, C₆₋₁₀ aryl, 4-6 membered heterocycloalkyl, 5 or 6-membered heteroaryl, C₁₋₄ haloalkyl, C₂₋₄ alkenyl, and C₂₋₄ alkynyl, wherein the C₁₋₄ alkyl, C₁₋₆ haloalkyl, C₃₋₆ cycloalkyl, C₆₋₁₀ aryl, 4-6 membered heterocycloalkyl, 5 or 6-membered heteroaryl, C₂₋₄ alkenyl, and C₂₋₄ alkynyl of Rⁱ, R^{k}, R^{o} or R^{r} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NHR¹² and NR¹²R¹², wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl and 4-6 membered heterocycloalkyl and each R¹² is independently C₁₋₆ alkyl;
the subscript n is an integer of 0, 1, 2, 3, 4, 5, 6, 7 or 8;
the subscript m is an integer of 0, 1, 2, 3, 4, 5, 6, 7 or 8;
each subscript p is independently an integer of 1, 2, 3 or 4;
each subscript t is independently an integer of 0, 1, 2, 3 or 4;
the subscript u is an integer of 0, 1, 2, 3, 4, 5, 6, 7 or 8;
with the proviso:
(i) when L¹ is a bond and 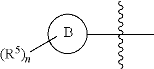
is phenyl or 2,3-dihydro-1,4-benzodioxin-6-yl, then 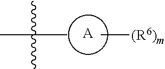
is not 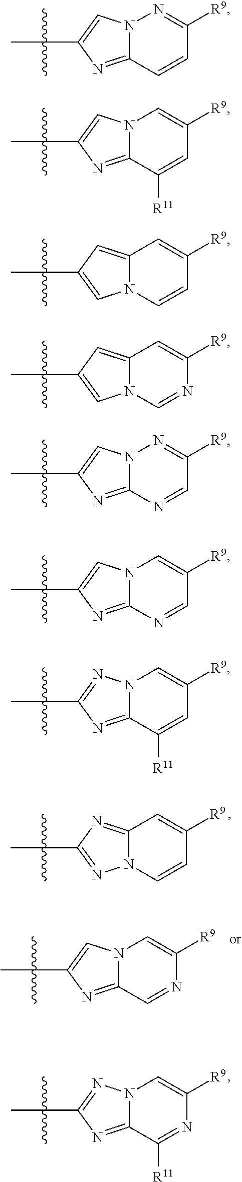
wherein each R⁹ is independently (2-hydroxyethylamino)methyl or (2-carboxy-1-piperidinyl)methyl and each R¹¹ is independently H, halo, CN, C₁₋₆ alkyl, C₁₋₆ alkoxy, —NHC₁₋₆ alkyl or benzyloxy, wherein the C₁₋₆ alkyl, C₁₋₆ alkoxy, —NHC₁₋₆ alkyl and benzyloxy of R¹¹ are each optionally substituted with halo, CN, C₁₋₆ alkyl or C₁₋₆ alkoxy;
(ii) when L¹ is a bond and 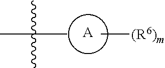
is phenyl or 2,3-dihydro-1,4-benzodioxin-6-yl, 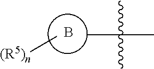
is not any of the moieties set forth in proviso (i) above for 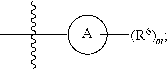
(iii) when L¹ is a bond, then 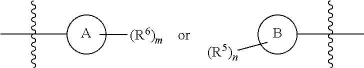
is not 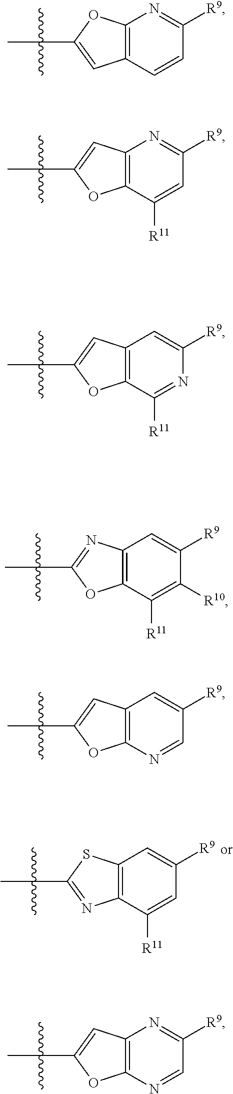
wherein each R⁹ is independently (2-hydroxyethylamino)methyl or (2-carboxy-1-piperidinyl)methyl; each R¹¹ is independently H or C₁₋₆ alkyl and R¹⁰ is H, C₁₋₆ alkoxy, benzyloxy, morpholinoethoxy or 2-pyridylmethyloxy, wherein the C₁₋₆ alkoxy, benzyloxy and 2-pyridylmethyloxy of R¹⁰ are each optionally substituted with CN;
(iv) when L¹ is a bond, then 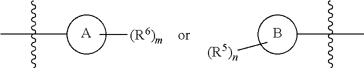
is not 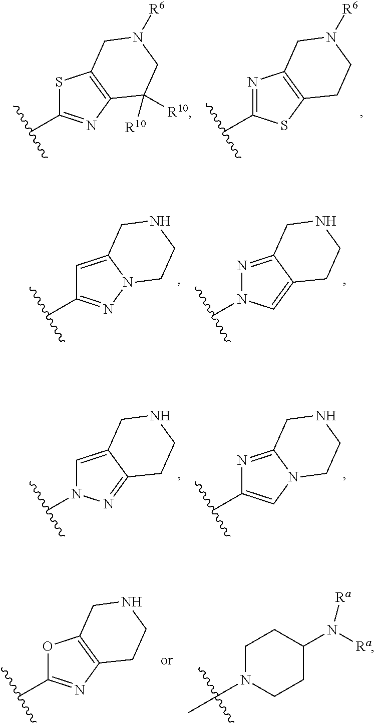
wherein R¹⁰ is H or C₁₋₆ alkyl;
(v) when L¹ is —NHC(O)—, then 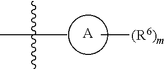
is not 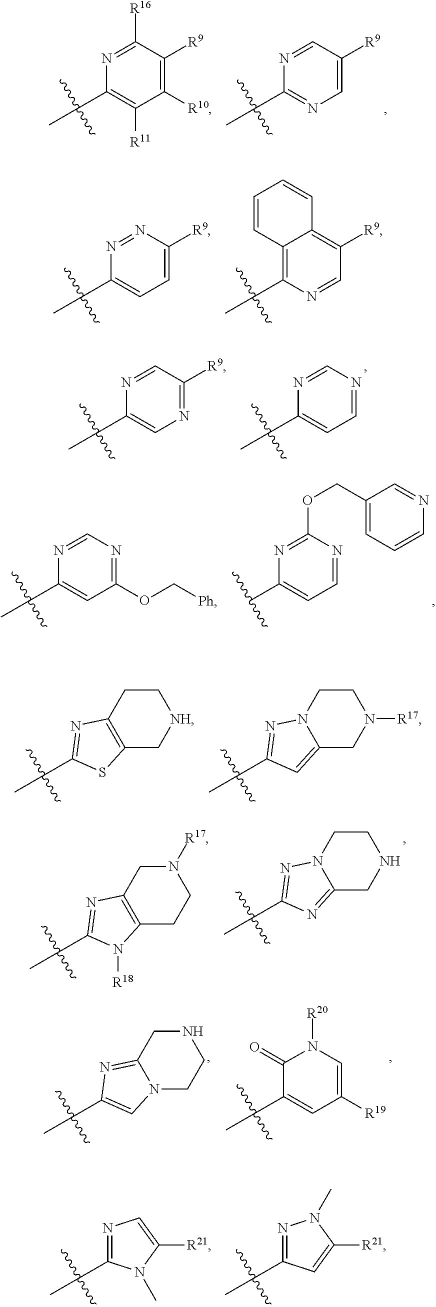 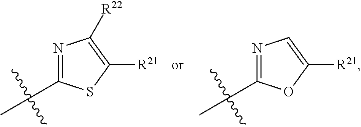
wherein each R⁹ is independently H, methyl, (2-hydroxyethylamino)methyl or (2-carboxy-1-piperidinyl)methyl; R¹⁰ is H, methyl, CN, methoxy, cyclopropylmethoxy, benzyloxy, (2-cyanophenyl)methoxy, 2-pyridylmethoxy, 3-pyridylmethoxy, (2-hydroxyethylamino)methyl or (2-carboxy-1-piperidinyl)methyl; R¹¹ is H, halo, methyl or dimethylamino; R¹⁶ is H or methyl; each R¹⁷ is independently H, 2-hydroxyethyl or carboxymethyl; R¹⁸ is H or methyl; R¹⁹ is (2-hydroxyethylamino)methyl or (2-carboxy-1-piperidinyl)methyl; R²⁰ is C₁₋₆ alkyl; each R²¹ is independently 2-hydroxyethylamino)methyl or (2-carboxy-1-piperidinyl)methyl; and R²² is H or Cl;
(vi) when L¹ is —NH— and 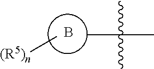
is phenyl, 2,3-dihydro-1,4-benzodioxin-6-yl, cyclohexyl or 1-cyclohexenyl, then 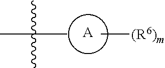
is not 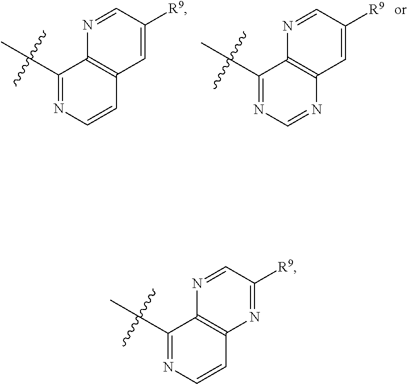
wherein each R⁹ is independently (2-hydroxyethylamino)methyl or (2-carboxy-1-piperidinyl)methyl;
(vii) when L¹ is —CH₂O—, ring B is phenyl or thienyl, and the subscript n is 1 or 2, then R⁵ is not a substituent independently selected from H, —OCH₃, —OH, —OCH₂CH₃, —O(CH₂)OCH₃, —OCH₂CH═CH₂, —O(CH₂)₂CH₃, —O(CH₂)₂morpholinyl or F; and
(viii) when L¹ is —CH₂O—, ring B is phenyl or thienyl, and the subscript n is 2, then two R⁵ substituents attached to adjacent ring carbon atoms of ring B do not form —OCH₂O— or —OCH₂CH₂O—; and
wherein the compound, or a pharmaceutically acceptable salt or a stereoisomer thereof inhibits PD-1/PD-L1 interaction.

2. The compound of claim 1, or a pharmaceutically acceptable salt or a stereoisomer thereof, wherein G has Formula (I′a) or (I′b):

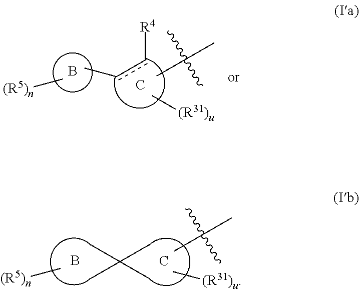

3. The compound of claim 1, or a pharmaceutically acceptable salt or a stereoisomer thereof,
when ring C is 4- to 14-membered heterocycloalkyl or C₃₋₁₄ cycloalkyl,
R⁴ is H, halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸,
wherein each R⁸ is independently H or C₁₋₆ alkyl.

4. The compound of claim 1, or a pharmaceutically acceptable salt or a stereoisomer thereof,
wherein R⁴ is halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸,
wherein each R⁸ is independently H or C₁₋₆ alkyl.

5. The compound of claim 1, having Formula (I′): 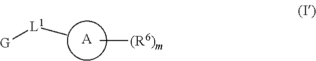
or a pharmaceutically acceptable salt or a stereoisomer thereof, wherein:
G has Formula (I′a) or (I′b) 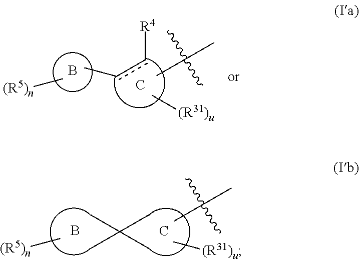
when G is of Formula (I′a), the atoms on ring C, to which the substituent R⁴ and ring B are attached can be either carbon or nitrogen; and [symbol] is a single bond or a double bond;
when G is of Formula (I′b), ring B and ring C are joined together through a quaternary ring carbon atom to form a spiro structure and ring B and ring C are each independently 4- to 14-membered heterocycloalkyl or C₃₋₁₄ cycloalkyl;
L¹ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³SO₂NR¹³(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;
ring A is C₆₋₁₀ aryl, 5- to 14-membered heteroaryl, 4- to 14-membered heterocycloalkyl or C₃₋₁₄ cycloalkyl;
ring B is C₆₋₁₀ aryl, 5- to 14-membered heteroaryl, 4- to 14-membered heterocycloalkyl or C₃₋₁₄ cycloalkyl;
ring C is C₆₋₁₀ aryl, 5- to 14-membered heteroaryl, 4- to 14-membered heterocycloalkyl or C₃₋₁₄ cycloalkyl;
each R¹³ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;
R¹⁴ and R¹⁵ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R¹⁴ or R¹⁵ are each optionally substituted with 1, 2, or 3 independently selected R^{q} substituents;
or R¹⁴ and R¹⁵ taken together with the carbon atom to which they are attached form C₃₋₆ cycloalkyl or 4- to 7-membered heterocycloalkyl, each of which is optionally substituted with 1, 2, or 3 independently selected R^{q} substituents;
R⁴ is halo, oxo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl, 5- to 6-membered heteroaryl, phenyl, or C₃₋₆ cycloalkyl, wherein the C₁₋₆ alkyl, C₁₋₆ alkoxy, C₂₋₆ alkenyl, C₂₋₆ alkynyl, 4- to 6-membered heterocycloalkyl, 5- to 6-membered heteroaryl, phenyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, 5- to 6-membered heteroaryl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl;
R⁵, R⁶ and R³¹ are each independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R⁵, R⁶ and R³¹ are each optionally substituted with 1, 2, 3, 4 or 5 independently selected R^{b} substituents;
or two adjacent R⁵ substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R⁵ substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
or two adjacent R⁶ substituents on ring A, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R⁶ substituents on the same ring carbon atom of the ring A, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
or two adjacent R³¹ substituents on ring C, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R³¹ substituents on the same ring carbon atom of ring C, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2 or 3 independently selected R^{d} substituents;
each R^{b} substituent is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1, 2, or 3 independently selected R^{d} substituents;
or two R^{b} substituents attached to the same ring carbon atom taken together with the ring carbon atom to which they are attached form spiro C₃₋₆ cycloalkyl or spiro 4- to 7-membered heterocycloalkyl, each of which is optionally substituted with 1, 2, or 3 independently selected R^{f} substituents;
each R^{c} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2 or 3 independently selected R^{f} substituents;
each R^{f} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2 or 3 independently selected Rⁿ substituents;
each Rⁿ is independently selected from C₁₋₆ alkyl, C₁₋₆ haloalkyl, halo, CN, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o}, wherein the C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of Rⁿ is optionally substituted with 1, 2 or 3 independently selected R^{q} substituents;
each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1, 2, or 3 independently selected R^{f} substituents;
each R^{e} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{e} are each optionally substituted with 1, 2 or 3 independently selected R^{f} substituents;
each R^{g} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1, 2, or 3 independently selected R^{p} substituents;
each R^{p} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r} and S(O)₂NR^{r}R^{r}, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} is optionally substituted with 1, 2 or 3 independently selected R^{q} substituents;
or any two R^{a} substituents together with the nitrogen atom to which they are attached form a 4-, 5-, 6-, 7-, 8-, 9- or 10-membered heterocycloalkyl group optionally substituted with 1, 2 or 3 independently selected R^{h} substituents;
each R^{h} is independently selected from C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, C₆₋₁₀ aryl-C₁₋₄ alkyl-, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, NRⁱC(═NOH)NRⁱRⁱ, NRⁱC(═NCN)NRⁱRⁱ, S(O)Rⁱ, S(O)NRⁱRⁱ, S(O)₂Rⁱ, NRⁱS(O)₂Rⁱ, NRⁱS(O)₂NRⁱRⁱ, and S(O)₂NRⁱRⁱ, wherein the C₁₋₆ alkyl, C₁₋₆ haloalkyl, C₁₋₆ haloalkoxy, C₃₋₁₀ cycloalkyl, 4-10 membered heterocycloalkyl, C₆₋₁₀ aryl, 5-10 membered heteroaryl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{h} are each optionally substituted by 1, 2, or 3 independently selected R^{j} substituents;
each R^{j} is independently selected from C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₂₋₄ alkynyl, halo, C₁₋₄ haloalkyl, C₁₋₄ haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR^{k})NR^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}, wherein the C₁₋₄ alkyl, C₃₋₁₀ cycloalkyl, C₆₋₁₀ aryl, 5-10 membered heteroaryl, 4-10 membered heterocycloalkyl, C₂₋₄ alkenyl, C₁₋₄ haloalkyl, and C₁₋₄ haloalkoxy of R^{j} are each optionally substituted with 1, 2 or 3 independently selected R^{q} substituents;
or two R^{h} groups attached to the same carbon atom of the 4- to 10-membered heterocycloalkyl, taken together with the carbon atom to which they are attached, form a C₃₋₆ cycloalkyl or 4- to 6-membered heterocycloalkyl having 1-2 heteroatoms as ring members selected from O, N or S;
or any two R^{c} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two R^{e} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two R^{g} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two Rⁱ substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two R^{k} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
or any two R^{o} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents; and
or any two R^{r} substituents together with the nitrogen atom to which they are attached form a 4-, 5-, 6-, 7-, 8-, 9-, or 10-membered heterocycloalkyl group optionally substituted with 1, 2, or 3 independently selected R^{h} substituents;
each Rⁱ, R^{k}, R^{o} or R^{r} is independently selected from H, C₁₋₆ alkyl, C₁₋₆ haloalkyl, C₃₋₆ cycloalkyl, C₆₋₁₀ aryl, 4-6 membered heterocycloalkyl, 5 or 6-membered heteroaryl, C₁₋₄ haloalkyl, C₂₋₄ alkenyl, and C₂₋₄ alkynyl, wherein the C₁₋₄ alkyl, C₁₋₆ haloalkyl, C₃₋₆ cycloalkyl, C₆₋₁₀ aryl, 4-6 membered heterocycloalkyl, 5 or 6-membered heteroaryl, C₂₋₄ alkenyl, and C₂₋₄ alkynyl of Rⁱ, R^{k}, R^{o} or R^{r} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NHR¹² and NR¹²R¹², wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl and 4-6 membered heterocycloalkyl and each R¹² is independently C₁₋₆ alkyl;
the subscript n is an integer of 0, 1, 2, 3, 4, 5, 6, 7 or 8;
the subscript m is an integer of 0, 1, 2, 3, 4, 5, 6, 7 or 8;
each subscript p is independently an integer of 1, 2, 3 or 4;
each subscript t is independently an integer of 0, 1, 2, 3 or 4;
the subscript u is an integer of 0, 1, 2, 3, 4, 5, 6, 7 or 8;
with the provisos:
(i) when L¹ is a bond and 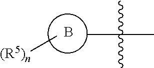
is phenyl or 2,3-dihydro-1,4-benzodioxin-6-yl, then 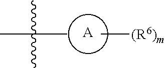
is not 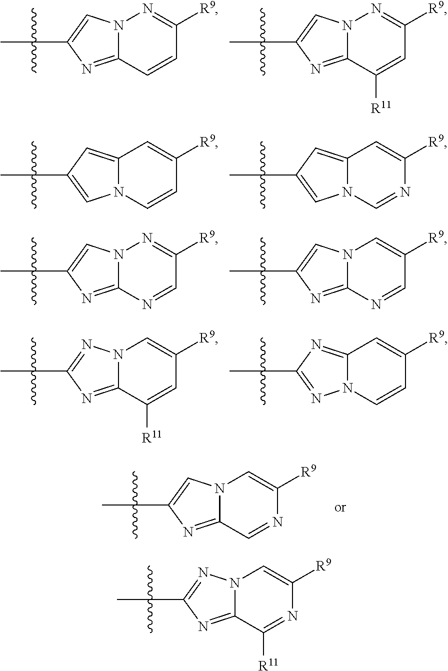
wherein each R⁹ is independently (2-hydroxyethylamino)methyl or (2-carboxy-1-piperidinyl)methyl and each R¹¹ is independently H, halo, CN, C₁₋₆ alkyl, C₁₋₆ alkoxy, —NHC₁₋₆ alkyl or benzyloxy, wherein the C₁₋₆ alkyl, C₁₋₆ alkoxy, —NHC₁₋₆ alkyl and benzyloxy of R¹¹ are each optionally substituted with halo, CN, C₁₋₆ alkyl or C₁₋₆ alkoxy;
(ii) when L¹ is a bond and 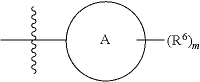
is phenyl or 2,3-dihydro-1,4-benzodioxin-6-yl, then 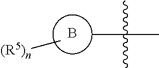
is not any of the moieties set forth in proviso (i) above for 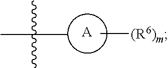
(iii) when L¹ is a bond then 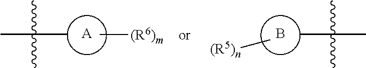
is not 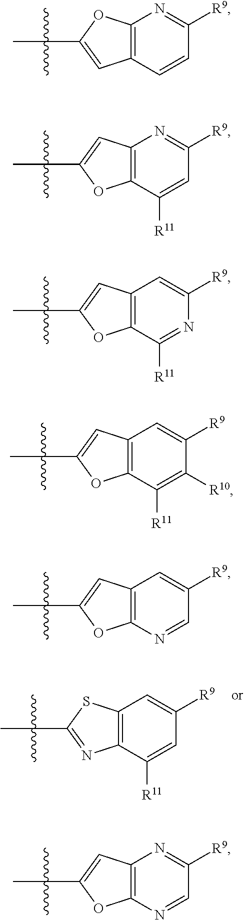
wherein each R⁹ is independently (2-hydroxyethylamino)methyl or (2-carboxy-1-piperidinyl)methyl; each R¹¹ is independently H or C₁₋₆ alkyl and R¹⁰ is H, C₁₋₆ alkoxy, benzyloxy, morpholinoethoxy or 2-pyridylmethyloxy, wherein the C₁₋₆ alkoxy, benzyloxy and 2-pyridylmethyloxy of R¹⁰ are each optionally substituted with CN;
(iv) when L¹ is a bond, then or 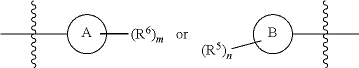
is not 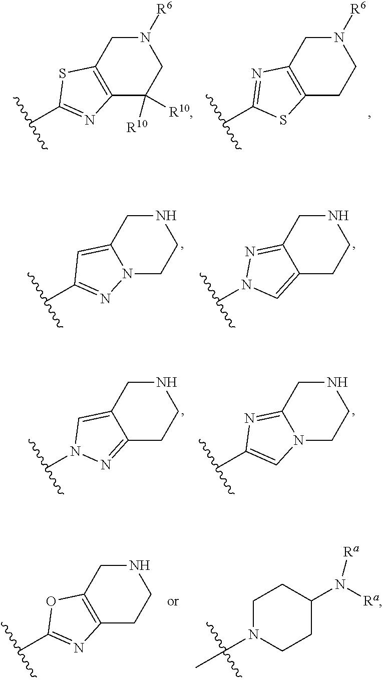
wherein R¹⁰ is H or C₁₋₆ alkyl;
(v) when L¹ is —NHC(O)—, then 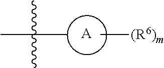
is not 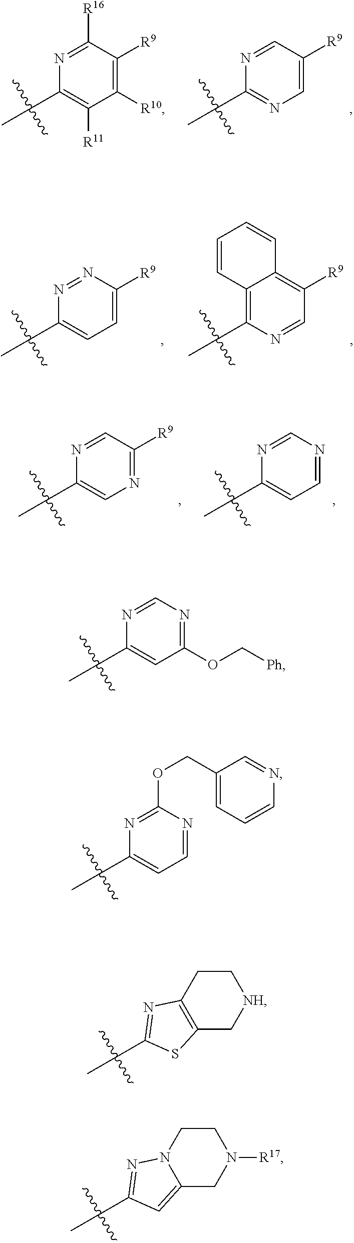 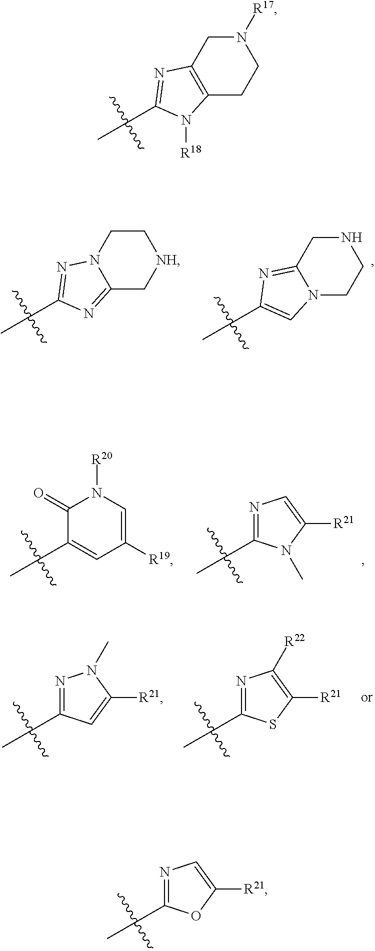
wherein each R⁹ is independently H, methyl, (2-hydroxyethylamino)methyl or (2-carboxy-1-piperidinyl)methyl; R¹⁰ is H, methyl, CN, methoxy, cyclopropylmethoxy, benzyloxy, (2-cyanophenyl)methoxy, 2-pyridylmethoxy, 3-pyridylmethoxy, (2-hydroxyethylamino)methyl or (2-carboxy-1-piperidinyl)methyl; R¹¹ is H, halo, methyl or dimethylamino; R¹⁶ is H or methyl; each R¹⁷ is independently H, 2-hydroxyethyl or carboxymethyl; R¹⁸ is H or methyl; R¹⁹ is (2-hydroxyethylamino)methyl or (2-carboxy-1-piperidinyl)methyl; R²⁰ is C₁₋₆ alkyl; each R²¹ is independently 2-hydroxyethylamino)methyl or (2-carboxy-1-piperidinyl)methyl; and R²² is H or Cl;
(vi) when L¹ is —NH— and 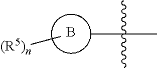
is phenyl, 2,3-dihydro-1,4-benzodioxin-6-yl, cyclohexyl or 1-cyclohexenyl, then 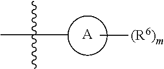
is not 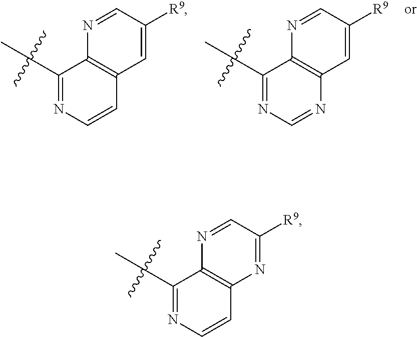
wherein each R⁹ is independently (2-hydroxyethylamino)methyl or (2-carboxy-1-piperidinyl)methyl;
(vii) when L¹ is —CH₂O—, ring B is phenyl or thienyl, and the subscript n is 1 or 2, then R⁵ is not a substituent independently selected from H, —OCH₃, —OH, —OCH₂CH₃, —O(CH₂)OCH₃, —OCH₂CH═CH₂, —O(CH₂)₂CH₃, —O(CH₂)₂morpholinyl or F; and
(viii) when L¹ is —CH₂O—, ring B is phenyl or thienyl, and the subscript n is 2, then two R⁵ substituents attached to adjacent ring carbon atoms of ring B do not form —OCH₂O— or —OCH₂CH₂O—; and
wherein the compound, or a pharmaceutically acceptable salt or a stereoisomer thereof inhibits PD-1/PD-L1 interaction.

6. The compound of claim 1, having Formula (I′c): 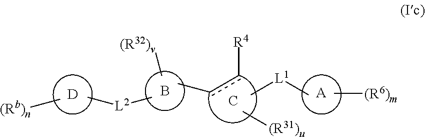
or a pharmaceutically acceptable salt or a stereoisomer thereof, wherein:
ring D is C₆₋₁₀ aryl, 5- to 14-membered heteroaryl, 4- to 14-membered heterocycloalkyl or C₃₋₁₄ cycloalkyl;
L² is a bond, —(CR²⁹R³⁰)ₜC(O)NR²⁸(CR²⁹R³⁰)ₜ—, —(CR²⁹R³⁰)ₜNR²⁸C(O)(CR²⁹R³⁰)ₜ—, O, —(CR²⁹R³⁰)_{q}—, —(CR²⁹R³⁰)_{q}—O—, —O(CR²⁹R³⁰)_{q}—, —(CR²⁹R³⁰)_{q}—O—(CR²⁹R³⁰)_{q}—, —NR²⁸—, —(CR²⁹R³⁰)_{w}NR²⁸(CR²⁹R³⁰)_{w}—, —NH—, —(CR²⁹R³⁰)_{w}NH(CR²⁹R³⁰)_{w}—, —CR²⁸═CR²⁸—, —C≡C—, —SO₂—, —(CR²⁹R³⁰)_{w}SO₂(CR²⁹R³⁰)_{w}—, —(CR²⁹R³⁰)_{w}SO₂NR²⁸(CR²⁹R³⁰)_{w}—, —(CR²⁹R³⁰)_{w}NR²⁸SO₂(CR²⁹R³⁰)_{w}—, —(CR²⁹R³⁰)_{w}NR²⁸SO₂NR²⁸(CR²⁹R³⁰)_{w}—, —(CR²⁹R³⁰)_{w}NR²⁸C(O)O(CR²⁹R³⁰)_{w}—, —NR²⁸C(O)O—, —(CR²⁹R³⁰)_{w}O(CO)NR²⁸(CR²⁹R³⁰)_{w}—, —O(CO)NR²⁸—, —NR²⁸C(O)NR²⁸— or —(CR²⁹R³⁰)_{w}NR²⁸C(O)NR²⁸(CR²⁹R³⁰)_{w};
each R²⁸ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;
R²⁹ and R³⁰ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R²⁹ or R³⁰ are each optionally substituted with 1, 2 or 3 independently selected R^{q} substituents;
or R²⁹ and R³⁰ taken together with the carbon atom to which they are attached form C₃₋₆ cycloalkyl or 4- to 7-membered heterocycloalkyl, each of which is optionally substituted with 1, 2, or 3 independently selected R^{q} substituents;
each R³² is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R³² are each optionally substituted with 1, 2, 3, 4 or 5 independently selected R^{b} substituents;
or two adjacent R³² substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
the subscript n is an integer of 0, 1, 2, 3, 4, 5;
the subscript v is an integer of 0, 1, 2, 3, 4, 5, 6 or 7
each subscript q is independently an integer of 1, 2, 3 or 4;
each subscript t is independently an integer of 0, 1, 2, 3 or 4; and
each subscript w is independently an integer of 0, 1, 2, 3 or 4.

7. The compound of claim 1, having Formula (I′d): 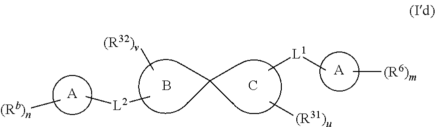
or a pharmaceutically acceptable salt or a stereoisomer thereof, wherein:
ring B and ring C are each independently 4- to 14-membered heterocycloalkyl or C₃₋₁₄ cycloalkyl;
ring D is C₆₋₁₀ aryl, 5- to 14-membered heteroaryl, 4- to 14-membered heterocycloalkyl or C₃₋₁₄ cycloalkyl;
L² is a bond, —(CR²⁹R³⁰)ₜC(O)NR²⁸(CR²⁹R³⁰)ₜ—, —(CR²⁹R³⁰)ₜNR²⁸C(O)(CR²⁹R³⁰)ₜ—, O, —(CR²⁹R³⁰)_{q}—, —(CR²⁹R³⁰)_{q}—O—, —O(CR²⁹R³⁰)_{q}—O—, —(CR²⁹R³⁰)_{q}—O—(CR²⁹R³⁰)_{q}—, —NR²⁸—, —(CR²⁹R³⁰)_{w}NR²⁸(CR²⁹R³⁰)_{w}—, —NH—, —(CR²⁹R³⁰)_{w}NH(CR²⁹R³⁰)_{w}—, —CR²⁸═CR²⁸—, —C≡C—, —SO₂—, —(CR²⁹R³⁰)_{w}SO₂(CR²⁹R³⁰)_{w}—, —(CR²⁹R³⁰)_{w}SO₂NR²⁸(CR²⁹R³⁰)_{w}—, —(CR²⁹R³⁰)_{w}NR²⁸SO₂(CR²⁹R³⁰)_{w}—, —(CR²⁹R³⁰)_{w}NR²⁸SO₂NR²⁸(CR²⁹R³⁰)_{w}—, —(CR²⁹R³⁰)_{w}NR²⁸C(O)O(CR²⁹R³⁰)_{w}—, —NR²⁸C(O)O—, —(CR²⁹R³⁰)_{w}O(CO)NR²⁸(CR²⁹R³⁰)_{w}—, —O(CO)NR²⁸—, —NR²⁸C(O)NR²⁸— or —(CR²⁹R³⁰)_{w}NR²⁸C(O)NR²⁸(CR²⁹R³⁰)_{w};
each R²⁸ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;
R²⁹ and R³⁰ are each independently selected from H, halo, CN, OH, NH₂, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R²⁹ or R³⁰ are each optionally substituted with 1, 2 or 3 independently selected R^{q} substituents;
or R²⁹ and R³⁰ taken together with the carbon atom to which they are attached form spiro C₃₋₆ cycloalkyl or spiro 4- to 7-membered heterocycloalkyl, each of which is optionally substituted with 1, 2, or 3 independently selected R^{q} substituents;
each R³² is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R³² are each optionally substituted with 1, 2, 3, 4 or 5 independently selected R^{b} substituents;
or two adjacent R³² substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R³² substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
the subscript n is an integer of 0, 1, 2, 3, 4, 5;
the subscript v is an integer of 0, 1, 2, 3, 4, 5, 6 or 7
each subscript q is independently an integer of 1, 2, 3 or 4;
each subscript t is independently an integer of 0, 1, 2, 3 or 4; and
each subscript w is independently an integer of 0, 1, 2, 3 or 4.

8. The compound of claim 1, having Formula (I): 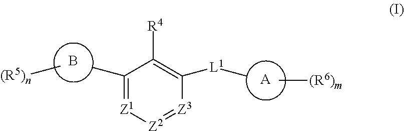
or a pharmaceutically acceptable salt or a stereoisomer thereof, wherein:
Z¹ is N or CR¹;
Z² is N or CR²;
Z³ is N or CR³;
L¹ is a bond, —(CR¹⁴R¹⁵)ₜC(O)NR¹³(CR¹⁴R¹⁵)ₜ—, —(CR¹⁴R¹⁵)ₜNR¹³C(O)(CR¹⁴R¹⁵)ₜ—, O, —(CR¹⁴R¹⁵)ₚ—, —(CR¹⁴R¹⁵)ₚ—O—, —O(CR¹⁴R¹⁵)ₚ—, —(CR¹⁴R¹⁵)ₚ—O—(CR¹⁴R¹⁵)ₚ—, —NR¹³—, —(CR¹⁴R¹⁵)ₜNR¹³(CR¹⁴R¹⁵)ₜ—, —NH—, —(CR¹⁴R¹⁵)ₜNH(CR¹⁴R¹⁵)ₜ—, —CR¹³═CR¹³—, —C≡C—, —SO₂—, —(CR¹⁴R¹⁵)ₜSO₂(CR¹⁴R¹⁵)ₜ—, —(CR¹⁴R¹⁵)ₜSO₂NR¹³(CR¹⁴R¹⁵)ₜ—, —(CR¹⁴R¹⁵)ₜNR¹³SO₂(CR¹⁴R¹⁵)ₜ—, —(CR¹⁴R¹⁵)ₜNR¹³C(O)O(CR¹⁴R¹⁵)ₜ—, —NR¹³C(O)O—, —(CR¹⁴R¹⁵)ₜO(CO)NR¹³(CR¹⁴R¹⁵)ₜ—, —O(CO)NR¹³—, —NR¹³C(O)NR¹³— or —(CR¹⁴R¹⁵)ₜNR¹³C(O)NR¹³(CR¹⁴R¹⁵)ₜ;
ring A is C₆₋₁₀ aryl, 5- to 14-membered heteroaryl, 4- to 11-membered heterocycloalkyl or C₃₋₁₀ cycloalkyl;
ring B is C₆₋₁₀ aryl, 5- to 14-membered heteroaryl, 4- to 11-membered heterocycloalkyl or C₃₋₁₀ cycloalkyl;
each R¹³ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;
R¹⁴ and R¹⁵ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R¹⁴ or R¹⁵ are each optionally substituted with 1, 2, or 3 independently selected R^{q} substituents;
or R¹⁴ and R¹⁵ taken together with the carbon atom to which they are attached form spiro C₃₋₆ cycloalkyl or spiro 4- to 7-membered heterocycloalkyl, each of which is optionally substituted with 1, 2, or 3 independently selected R^{q} substituents;
R¹, R² and R³ are each independently selected from H, C₁₋₄ alkyl, C₃₋₁₀ cycloalkyl, C₃₋₁₀ cycloalkyl-C₁₋₄ alkyl-, C₆₋₁₀ aryl, C₆₋₁₀ aryl-C₁₋₄ alkyl-, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, C₂₋₄ alkenyl, C₂₋₄ alkynyl, halo, CN, OR⁷, C₁₋₄ haloalkyl, C₁₋₄ haloalkoxy, NH₂, —NHR⁷, —NR⁷R⁷, NHOR⁷, C(O)R⁷, C(O)NR⁷R⁷, C(O)OR⁷, OC(O)R⁷, OC(O)NR⁷R⁷, NR⁷C(O)R⁷, NR⁷C(O)OR⁷, NR⁷C(O)NR⁷R⁷, C(═NR⁷)R⁷, C(═NR⁷)NR⁷R⁷, NR⁷C(═NR⁷)NR⁷R⁷, NR⁷S(O)R⁷, NR⁷S(O)₂R⁷, NR⁷S(O)₂NR⁷R⁷, S(O)R⁷, S(O)NR⁷R⁷, S(O)₂R⁷, and S(O)₂NR⁷R⁷, wherein each R⁷ is independently selected from H, C₁₋₄ alkyl, C₂₋₄ alkenyl, C₂₋₄ alkynyl, C₁₋₄ alkoxy, C₃₋₆ cycloalkyl, C₃₋₁₀ cycloalkyl-C₁₋₄ alkyl-, C₆₋₁₀ aryl, C₆₋₁₀ aryl-C₁₋₄ alkyl, 5-10 membered heteroaryl, 4-10 membered heterocycloalkyl, (5-10 membered heteroaryl)-C₁₋₄ alkyl-, and (4-10 membered heterocycloalkyl)-C₁₋₄ alkyl-, wherein the C₁₋₄ alkyl, C₁₋₄ alkoxy, C₃₋₁₀ cycloalkyl, C₃₋₆ cycloalkyl-C₁₋₄ alkyl-, C₆₋₁₀ aryl, C₆₋₁₀ aryl-C₁₋₄ alkyl-, 5-10 membered heteroaryl, 4-10 membered heterocycloalkyl, (5-10 membered heteroaryl)-C₁₋₄ alkyl-, and (4-10 membered heterocycloalkyl)-C₁₋₄ alkyl- of R¹, R², R³ and R⁷ are each optionally substituted with 1 or 2 independently selected R^{d} substituents;
R⁴ is halo, CN, C₁₋₆ alkyl, C₁₋₆ alkoxy, C₂₋₆ alkenyl, C₂₋₆ alkynyl, C₁₋₆ haloalkyl, C₁₋₆ haloalkoxy, 4 to 6-membered heterocycloalkyl or C₃₋₆ cycloalkyl, wherein the C₁₋₆ alkyl, C₁₋₆ alkoxy, C₂₋₆ alkenyl, C₂₋₆ alkynyl, 4 to 6-membered heterocycloalkyl and C₃₋₆ cycloalkyl are each optionally substituted with 1, 2 or 3 substituents independently selected from halo, CN, C₁₋₆ alkoxy, C₁₋₆ haloalkyl, C₁₋₆ haloalkoxy, 4 to 6-membered heterocycloalkyl, C₃₋₆ cycloalkyl, phenyl, NH₂, —NHR⁸, —NR⁸R⁸, C(O)R⁸, C(O)NR⁸R⁸, OC(O)NR⁸R⁸, NR⁸C(O)R⁸, NR⁸C(O)OR⁸, NR⁸C(O)NR⁸R⁸, NR⁸S(O)₂R⁸, NR⁸S(O)₂NR⁸R⁸, S(O)R⁸, S(O)₂R⁸, and S(O)₂NR⁸R⁸, wherein each R⁸ is independently H or C₁₋₆ alkyl;
R⁵ and R⁶ are each independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R⁵ and R⁶ are each optionally substituted with 1, 2, 3, 4 or 5 independently selected R^{b} substituents;
or two adjacent R⁵ substituents on ring B, taken together with the atoms to which they are attached, form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused 4-, 5-, 6- or 7-membered heterocycloalkyl ring and fused 5- or 6-membered heteroaryl ring each have 1-4 heteroatoms as ring members selected from N, O and S and wherein the fused phenyl ring, fused 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R⁵ substituents on the same ring carbon atom of ring B, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
or two adjacent R⁶ substituents on ring A, taken together with the atoms to which they are attached, form a fused phenyl ring, a fused 5-, 6- or 7-membered heterocycloalkyl ring, a fused 5- or 6-membered heteroaryl ring or a fused C₃₋₆ cycloalkyl ring, wherein the fused 5-, 6- or 7-membered heterocycloalkyl ring and fused 5- or 6-membered heteroaryl ring each have 1-4 heteroatoms as ring members selected from N, O and S and wherein the fused phenyl ring, fused 5-, 6- or 7-membered heterocycloalkyl ring, fused 5- or 6-membered heteroaryl ring and fused C₃₋₆ cycloalkyl ring are each optionally substituted with 1, 2 or 3 independently selected R^{b} substituents;
or two R⁶ substituents on the same ring carbon atom of the ring A, taken together with the carbon atom to which they are attached, form a spiro 4-, 5-, 6- or 7-membered heterocycloalkyl ring, or a spiro C₃₋₆ cycloalkyl ring, wherein the spiro 4-, 5-, 6- or 7-membered heterocycloalkyl ring has 1-4 heteroatoms as ring members selected from N, O and S and wherein the spiro 4-, 5-, 6- or 7-membered heterocycloalkyl ring and spiro C₃₋₆ cycloalkyl ring are each optionally substituted with 1, 2 or 3 independently selected R^{b} substituents;
each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2 or 3 independently selected R^{d} substituents;
each R^{b} substituent is independently selected from halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1, 2, or 3 independently selected R^{d} substituents;
or two R^{b} substituents attached to the same ring carbon atom taken together with the ring carbon atom to which they are attached form spiro C₃₋₆ cycloalkyl or spiro 4- to 7-membered heterocycloalkyl, each of which is optionally substituted with 1, 2, or 3 independently selected R^{f} substituents;
each R^{c} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2 or 3 R^{f} substituents;
each R^{f} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2 or 3 Rⁿ substituents;
each Rⁿ is independently selected from C₁₋₆ alkyl, C₁₋₆ haloalkyl, halo, CN, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o}, wherein the C₁₋₆ alkyl, C₁₋₆ haloalkyl, phenyl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of Rⁿ is optionally substituted with 1, 2 or 3 R^{q} substituents;
each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1, 2, or 3 independently selected R^{f} substituents;
each R^{e} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{e} are each optionally substituted with 1, 2 or 3 independently selected R^{f} substituents;
each R^{g} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1, 2, or 3 R^{p} substituents;
each R^{p} is independently selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r} and S(O)₂NR^{r}R^{r}, wherein the C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} is optionally substituted with 1, 2 or 3 R^{q} substituents;
or any two R^{a} substituents together with the nitrogen atom to which they are attached form a 4-, 5-, 6-, 7-, 8-, 9- or 10-membered heterocycloalkyl group optionally substituted with 1, 2 or 3 R^{h} substituents;
each R^{h} is independently selected from C₁₋₆ alkyl, C₁₋₆ haloalkyl, C₁₋₆ haloalkoxy, C₃₋₁₀ cycloalkyl, 4-7 membered heterocycloalkyl, C₆₋₁₀ aryl, 5-6 membered heteroaryl, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₆₋₁₀ aryl-C₁₋₄ alkyl-, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, NRⁱC(═NOH)NRⁱRⁱ, NRⁱC(═NCN)NRⁱRⁱ, S(O)Rⁱ, S(O)NRⁱRⁱ, S(O)₂Rⁱ, NR'S(O)₂Rⁱ, NR'S(O)₂NR^{I}Rⁱ, and S(O)₂NRⁱRⁱ, wherein the C₁₋₆ alkyl, C₁₋₆ haloalkyl, C₁₋₆ haloalkoxy, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, and (4-7 membered heterocycloalkyl)-C₁₋₄ alkyl- of R^{h} are each optionally substituted by 1, 2, or 3 R^{j} substituents;
each R^{j} is independently selected from C₁₋₄ alkyl, C₃₋₆ cycloalkyl, C₆₋₁₀ aryl, 5- or 6-membered heteroaryl, 4-6 membered heterocycloalkyl, C₂₋₄ alkenyl, C₂₋₄ alkynyl, halo, C₁₋₄ haloalkyl, C₁₋₄ haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR^{k})NR^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}, wherein the C₁₋₄ alkyl, C₃₋₆ cycloalkyl, C₆₋₁₀ aryl, 5- or 6-membered heteroaryl, 4-6 membered heterocycloalkyl, C₂₋₄ alkenyl, C₁₋₄ haloalkyl, and C₁₋₄ haloalkoxy of R^{j} are each optionally substituted with 1, 2 or 3 independently selected R^{q} substituents;
or two R^{h} groups attached to the same carbon atom of the 4- to 10-membered heterocycloalkyl, taken together with the carbon atom to which they are attached, form a C₃₋₆ cycloalkyl or 4- to 6-membered heterocycloalkyl having 1-2 heteroatoms as ring members selected from O, N or S;
or any two R^{c} substituents together with the nitrogen atom to which they are attached form a 4-, 5-, 6-, or 7-membered heterocycloalkyl group optionally substituted with 1, 2, or 3 independently selected R^{h} substituents;
or any two R^{e} substituents together with the nitrogen atom to which they are attached form a 4-, 5-, 6-, or 7-membered heterocycloalkyl group optionally substituted with 1, 2, or 3 independently selected R^{h} substituents;
or any two R^{g} substituents together with the nitrogen atom to which they are attached form a 4-, 5-, 6-, or 7-membered heterocycloalkyl group optionally substituted with 1, 2, or 3 independently selected R^{h} substituents;
or any two Rⁱ substituents together with the nitrogen atom to which they are attached form a 4-, 5-, 6-, or 7-membered heterocycloalkyl group optionally substituted with 1, 2, or 3 independently selected R^{h} substituents;
or any two R^{k} substituents together with the nitrogen atom to which they are attached form a 4-, 5-, 6-, or 7-membered heterocycloalkyl group optionally substituted with 1, 2, or 3 independently selected R^{h} substituents;
or any two R^{o} substituents together with the nitrogen atom to which they are attached form a 4-, 5-, 6-, or 7-membered heterocycloalkyl group optionally substituted with 1, 2, or 3 independently selected R^{h} substituents; and
or any two R^{r} substituents together with the nitrogen atom to which they are attached form a 4-, 5-, 6-, or 7-membered heterocycloalkyl group optionally substituted with 1, 2, or 3 independently selected R^{h} substituents;
each Rⁱ, R^{k}, R^{o} or R^{r} is independently selected from H, C₁₋₆ alkyl, C₁₋₆ haloalkyl, C₃₋₆ cycloalkyl, C₆₋₁₀ aryl, 4-6 membered heterocycloalkyl, 5 or 6-membered heteroaryl, C₁₋₄ haloalkyl, C₂₋₄ alkenyl, and C₂₋₄ alkynyl, wherein the C₁₋₄ alkyl, C₁₋₆ haloalkyl, C₃₋₆ cycloalkyl, C₆₋₁₀ aryl, 4-6 membered heterocycloalkyl, 5 or 6-membered heteroaryl, C₂₋₄ alkenyl, and C₂₋₄ alkynyl of Rⁱ, R^{k}, R^{o} or R^{r} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NHR¹² and NR¹²R¹², wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl and 4-6 membered heterocycloalkyl and each R¹² is independently C₁₋₆ alkyl;
the subscript n is an integer of 0, 1, 2, 3, 4, 5, 6, 7 or 8;
the subscript m is an integer of 0, 1, 2, 3, 4, 5, 6, 7 or 8;
each subscript p is independently an integer of 1, 2, 3 or 4;
each subscript t is independently an integer of 0, 1, 2, 3 or 4;
with the provisos:
(i) when L¹ is a bond and 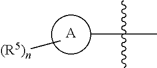
is phenyl or 2,3-dihydro-1,4-benzodioxin-6-yl, then 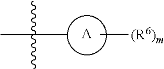
is not 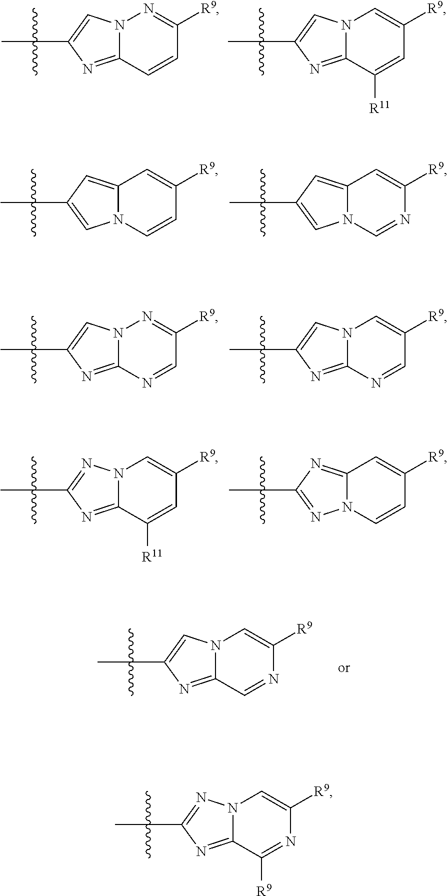
wherein each R⁹ is independently (2-hydroxyethylamino)methyl or (2-carboxy-1-piperidinyl)methyl and each R¹¹ is independently H, halo, CN, C₁₋₆ alkyl, C₁₋₆ alkoxy, —NHC₁₋₆ alkyl or benzyloxy, wherein the C₁₋₆ alkyl, C₁₋₆ alkoxy, —NHC₁₋₆ alkyl and benzyloxy of R¹¹ are each optionally substituted with halo, CN, C₁₋₆ alkyl or C₁₋₆ alkoxy;
(ii) when L¹ is a bond and 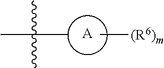
is phenyl or 2,3-dihydro-1,4-benzodioxin-6-yl, then 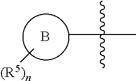
is not any of the moieties set forth in proviso (i) above for 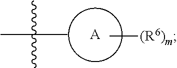
(iii) when L¹ is a bond, then 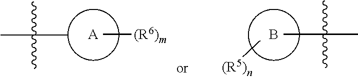
or is not 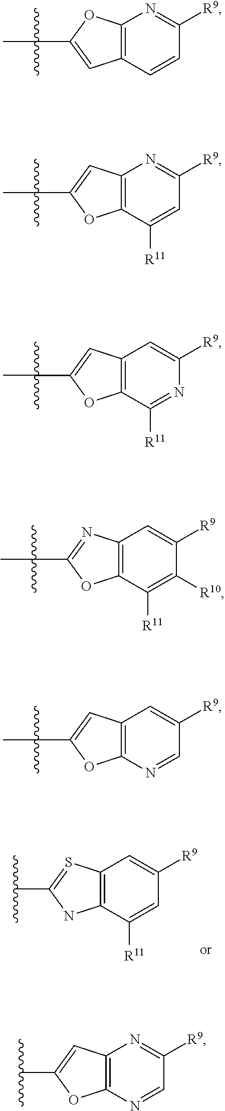
wherein each R⁹ is independently (2-hydroxyethylamino)methyl or (2-carboxy-1-piperidinyl)methyl; each R¹¹ is independently H or C₁₋₆ alkyl and R¹⁰ is H, C₁₋₆ alkoxy, benzyloxy, morpholinoethoxy or 2-pyridylmethyloxy, wherein the C₁₋₆ alkoxy, benzyloxy and 2-pyridylmethyloxy of R¹⁰ are each optionally substituted with CN;
(iv) when L¹ is a bond, then 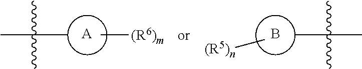
is not 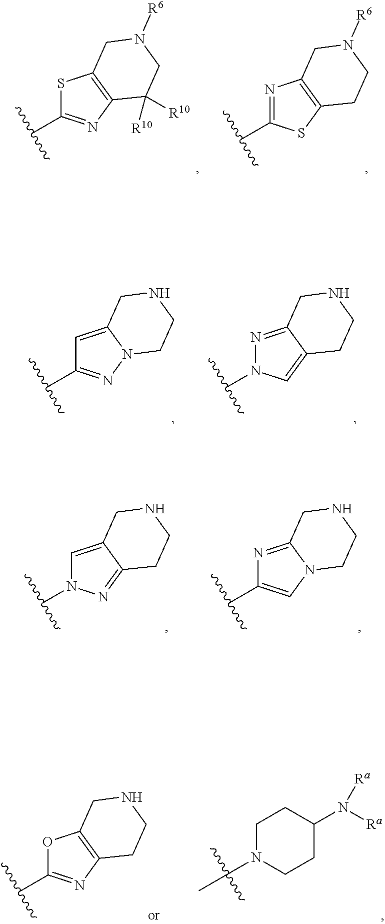
wherein R¹⁰ is H or C₁₋₆ alkyl;
(v) when L¹ is —NHC(O)—, then 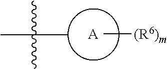
is not 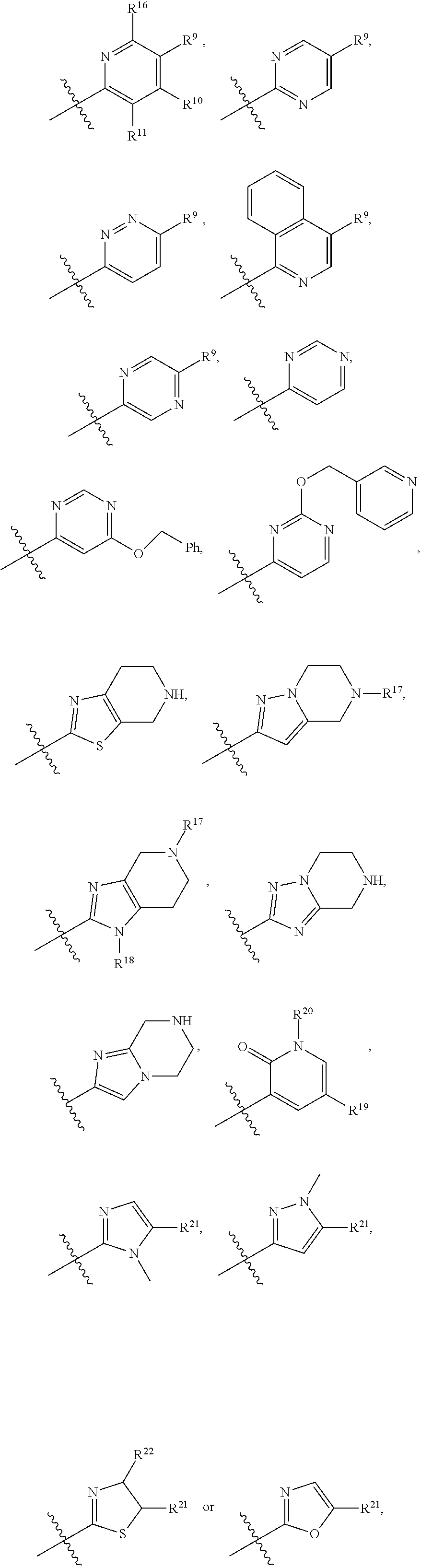
wherein each R⁹ is independently H, methyl, (2-hydroxyethylamino)methyl or (2-carboxy-1-piperidinyl)methyl; R¹⁰ is H, methyl, CN, methoxy, cyclopropylmethoxy, benzyloxy, (2-cyanophenyl)methoxy, 2-pyridylmethoxy, 3-pyridylmethoxy, (2-hydroxyethylamino)methyl or (2-carboxy-1-piperidinyl)methyl; R¹¹ is H, halo, methyl or dimethylamino; R¹⁶ is H or methyl; each R¹⁷ is independently H, 2-hydroxyethyl or carboxymethyl; R¹⁸ is H or methyl; R¹⁹ is (2-hydroxyethylamino)methyl or (2-carboxy-1-piperidinyl)methyl; R²⁰ is C₁₋₆ alkyl; each R²¹ is independently 2-hydroxyethylamino)methyl or (2-carboxy-1-piperidinyl)methyl; and R²² is H or Cl;
(vi) when L¹ is —NH— and 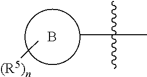
is phenyl, 2,3-dihydro-1,4-benzodioxin-6-yl, cyclohexyl or 1-cyclohexenyl, then 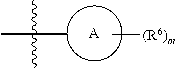
is not 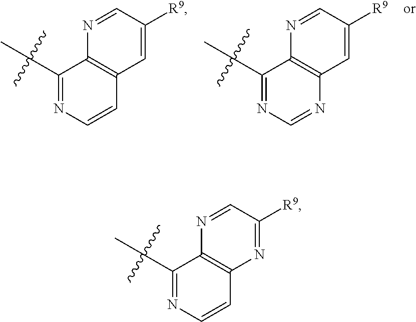
wherein each R⁹ is independently (2-hydroxyethylamino)methyl or (2-carboxy-1-piperidinyl)methyl;
(vii) when L¹ is —CH₂O—, ring B is phenyl or thienyl, and the subscript n is 1 or 2, then R⁵ is not a substituent independently selected from H, —OCH₃, —OH, —OCH₂CH₃, —O(CH₂)OCH₃, —OCH₂CH═CH₂, —O(CH₂)₂CH₃, —O(CH₂)₂morpholinyl or F; and
(viii) when L¹ is —CH₂O—, ring B is phenyl or thienyl, and the subscript n is 2, then two R⁵ substituents attached to adjacent ring carbon atoms of ring B do not form —OCH₂O— or —OCH₂CH₂O—; and
wherein the compound, or a pharmaceutically acceptable salt or a stereoisomer thereof inhibits PD-1/PD-L1 interaction.

9. The compound of claim 1, or a pharmaceutically acceptable salt or a stereoisomer thereof, wherein:
any two Rⁱ substituents together with the nitrogen atom to which they are attached form a 4-, 5-, 6-, 7-, 8-, 9- or 10-membered heterocycloalkyl group optionally substituted with 1, 2, or 3 independently selected R^{q} substituents;
or any two R^{k} substituents together with the nitrogen atom to which they are attached form a 4-, 5-, 6-, 7-, 8-, 9- or 10-membered heterocycloalkyl group optionally substituted with 1, 2, or 3 independently selected R^{q} substituents.

10. The compound of claim 1, or a pharmaceutically acceptable salt or a stereoisomer thereof, having an IC₅₀ of less than 1 μM in a PD-L1 binding assay.

11. The compound of claim 1, or a pharmaceutically acceptable salt or a stereoisomer thereof, wherein
the subscript m is an integer of 0, 1, 2, 3, 4, 5, 6, 7 or 8 and the subscript n is an integer of 1, 2, 3, 4, 5, 6, 7 or 8;
or the subscript m is an integer of 1, 2, 3, 4, 5, 6, 7 or 8 and the subscript n is an integer of 0, 1, 2, 3, 4, 5, 6, 7 or 8;
or the subscripts m and n are each independently an integer of 1, 2, 3, 4, 5, 6, 7 or 8.

12. The compound of claim 1, or a pharmaceutically acceptable salt or a stereoisomer thereof, wherein

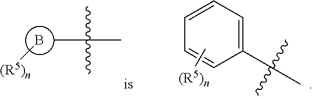

13. The compound of claim 1, having Formula (II): 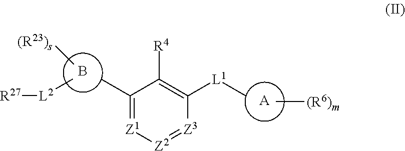
or a pharmaceutically acceptable salt or a stereoisomer thereof, wherein:
L² is a bond, —(CR²⁹R³⁰)ₜC(O)NR²⁸(CR²⁹R³⁰)ₜ—, —(CR²⁹R³⁰)ₜNR²⁸C(O)(CR²⁹R³⁰)ₜ—, O, —(CR²⁹R³⁰)_{q}—, —(CR²⁹R³⁰)_{q}—O—, —O(CR²⁹R³⁰)_{q}—, —(CR²⁹R³⁰)_{q}—O—(CR²⁹R³⁰)_{q}—, —NR²⁸—, —(CR²⁹R³⁰)ₜNR²⁸(CR²⁹R³⁰)ₜ—, —NH—, —(CR²⁹R³⁰)ₜNH(CR²⁹R³⁰)ₜ—, —CH═CH—, —C≡C—, —SO₂—, —(CR²⁹R³⁰)ₜSO₂(CR²⁹R³⁰)ₜ—, —(CR²⁹R³⁰)ₜSO₂NR²⁸(CR²⁹R³⁰)ₜ—, —(CR²⁹R³⁰)ₜNR²⁸SO₂(CR²⁹R³⁰)ₜ—, —(CR²⁹R³⁰)ₜNR²⁸C(O)O(CR²⁹R³⁰)ₜ—, —NR²⁸C(O)O—, —(CR²⁹R³⁰)ₜO(CO)NR²⁸(CR²⁹R³⁰)ₜ—, —O(CO)NR²⁸—, —NR²⁸C(O)NR²⁸— or —(CR²⁹R³⁰)ₜNR²⁸C(O)NR²⁸(CR²⁹R³⁰)ₜ;
each R²³ is independently C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl or —N(C₁₋₄ alkyl)₂;
R²⁷ is C₆₋₁₀ aryl, C₃₋₁₀ cycloalkyl, 5-14 membered heteroaryl, 4-11 membered heterocycloalkyl, each of which is optionally substituted with 1, 2, 3, 4 or 5 independently selected R^{b} substituents;
each R²⁸ is independently H, C₁₋₆ haloalkyl or C₁₋₆ alkyl optionally substituted with a substituent selected from C₁₋₄ alkyl, C₁₋₄ alkoxy, C₁₋₄ haloalkyl, C₁₋₄ haloalkoxy, CN, halo, OH, —COOH, NH₂, —NHC₁₋₄ alkyl and —N(C₁₋₄ alkyl)₂;
R²⁹ and R³⁰ are each independently selected from H, halo, CN, OH, —COOH, C₁₋₄ alkyl, C₁₋₄ alkoxy, —NHC₁₋₄ alkyl, —N(C₁₋₄ alkyl)₂, C₁₋₄ haloalkyl, C₁₋₄ haloalkoxy, C₃₋₆ cycloalkyl, phenyl, 5-6 membered heteroaryl and 4-6 membered heterocycloalkyl, wherein the C₁₋₄ alkyl, C₁₋₄ alkoxy, C₁₋₄ haloalkyl, C₁₋₄ haloalkoxy, C₃₋₆ cycloalkyl, phenyl, 5-6 membered heteroaryl and 4-6 membered heterocycloalkyl of R²⁹ or R³⁰ are each optionally substituted with 1, 2 or 3 independently selected R^{q} substituents;
or R²⁹ and R³⁰ taken together with the carbon atom to which they are attached form spiro C₃₋₆ cycloalkyl or spiro 4- to 7-membered heterocycloalkyl, each of which is optionally substituted with 1, 2, or 3 independently selected R^{q} substituents;
the subscript s is an integer of 0, 1, 2, 3 or 4; and
each subscript q is independently an integer of 1, 2, 3 or 4.

14. The compound of claim 13, having Formula (IIa): 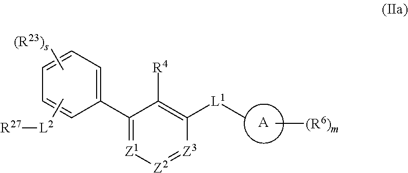
or a pharmaceutically acceptable salt or a stereoisomer thereof.

15. The compound of claim 8, having Formula (III): 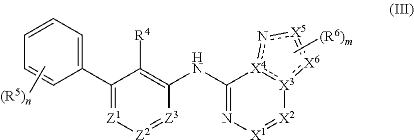
or a pharmaceutically acceptable salt or a stereoisomer thereof, wherein:
X¹, X², X³, X⁴ and X⁶ are each independently C or N, with the proviso that no more than two of X¹, X², X³ and X⁴ are simultaneously N;
X⁵ is C, N, O or S; [symbol]
is a single or a double bond to maintain the fused 5- and 6-membered rings being aromatic.

16. The compound of claim 8, having Formula (IV): 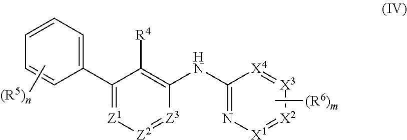
or a pharmaceutically acceptable salt or a stereoisomer thereof, wherein:
X¹, X², X³ and X⁴ are each independently C or N, with the proviso that no more than two of X¹, X², X³ and X⁴ are simultaneously N.

17. The compound of claim 8, having Formula (V): 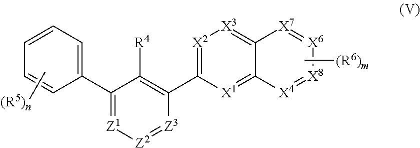
or a pharmaceutically acceptable salt or a stereoisomer thereof, wherein:
X¹, X², X³, X⁴, X⁶, X⁷ and X⁸ are each independently C or N, with the proviso that no more than three of X⁴, X⁶, X⁷ and X⁸ are simultaneously N.

18. The compound of claim 8, having Formula (VI): 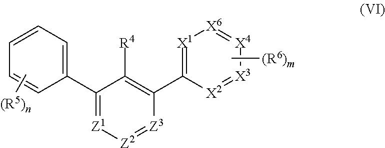
or a pharmaceutically acceptable salt or a stereoisomer thereof, wherein:
X¹, X², X³, X⁴ and X⁶ are each independently C or N, with the proviso that no more than three of X¹, X², X³, X⁴ and X⁶ are simultaneously N.

19. The compound of claim 8, having Formula (VII): 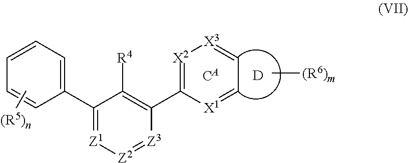
or a pharmaceutically acceptable salt or a stereoisomer thereof, wherein:
X¹, X² and X³ are each independently C or N; and
ring C^{A} is aromatic and ring D is fused 5- or 6-membered heterocycloalkyl.

20. The compound of claim 8, having Formula (VIII): 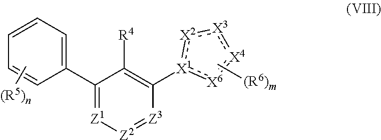
or a pharmaceutically acceptable salt or a stereoisomer thereof, wherein:
X¹ is N or C;
X², X³, X⁴ and X⁶ are each independently C, N, or O to maintain the 5-membered ring A being aromatic.

21. The compound of claim 8, having Formula (IX): 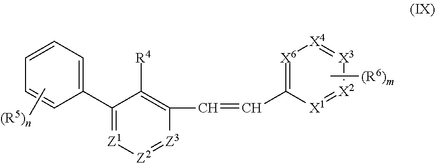
or a pharmaceutically acceptable salt or a stereoisomer thereof, wherein:
X¹, X² and X³ are each independently C or N;
X⁴ is CR²⁴ or N;
X⁶ is CR²⁵ or N;
or R²⁴ and R²⁵ together with the carbon atoms to which they are attached form a fused phenyl ring, a fused 4-, 5-, 6- or 7-membered heterocycloalkyl ring, a fused 5- or 6-membered heteroaryl ring or a fused C₃₋₆ cycloalkyl ring, wherein the fused phenyl ring, fused 4-, 5-, 6- or 7-membered heterocycloalkyl ring, fused 5- or 6-membered heteroaryl ring and fused C₃₋₆ cycloalkyl ring are each optionally substituted with 1, 2 or 3 R^{b} substituents.

22. The compound of claim 8, having Formula (X): 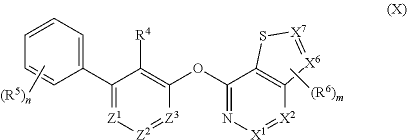
or a pharmaceutically acceptable salt or a stereoisomer thereof, wherein:
X¹, X², X⁶ and X⁷ are each independently C or N.

23. The compound of claim 8, or a pharmaceutically acceptable salt or a stereoisomer thereof, wherein Z¹ is CR¹, Z² is CR² and Z³ is CR³.

24. The compound of claim 1, or a pharmaceutically acceptable salt or a stereoisomer thereof, wherein 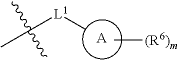
is selected from 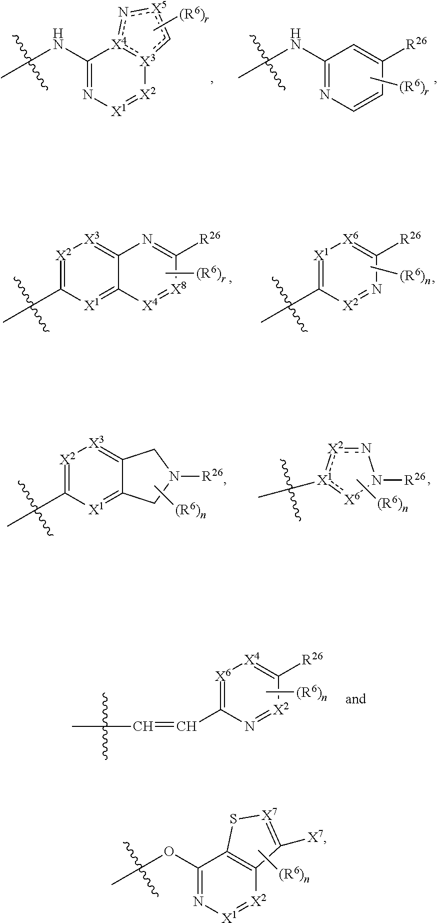
wherein
X¹, X², X³, X⁴, X⁶, X⁷ and X⁸ are each independently C or N;
X⁵ is C, N, O or S;
each R²⁶ is independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R²⁶ is optionally substituted with 1, 2, 3, 4 or 5 independently selected R^{b} substituents; and
each subscript r is independently an integer of 1, 2, 3, 4, 5, 6 or 7.

25. The compound of claim 1, or a pharmaceutically acceptable salt or a stereoisomer thereof, wherein 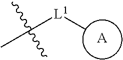
is selected from: 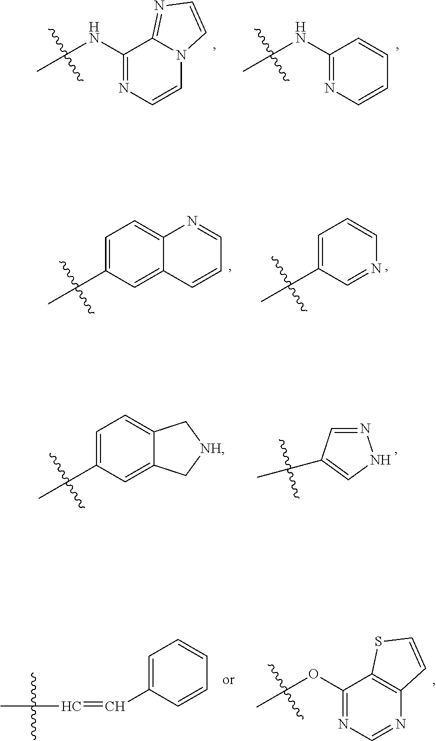
each of which is optionally substituted with 1 to 5 independently selected R⁶ substituents.

26. The compound of claim 1, or a pharmaceutically acceptable salt or a stereoisomer thereof, wherein 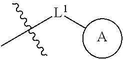
is selected from: 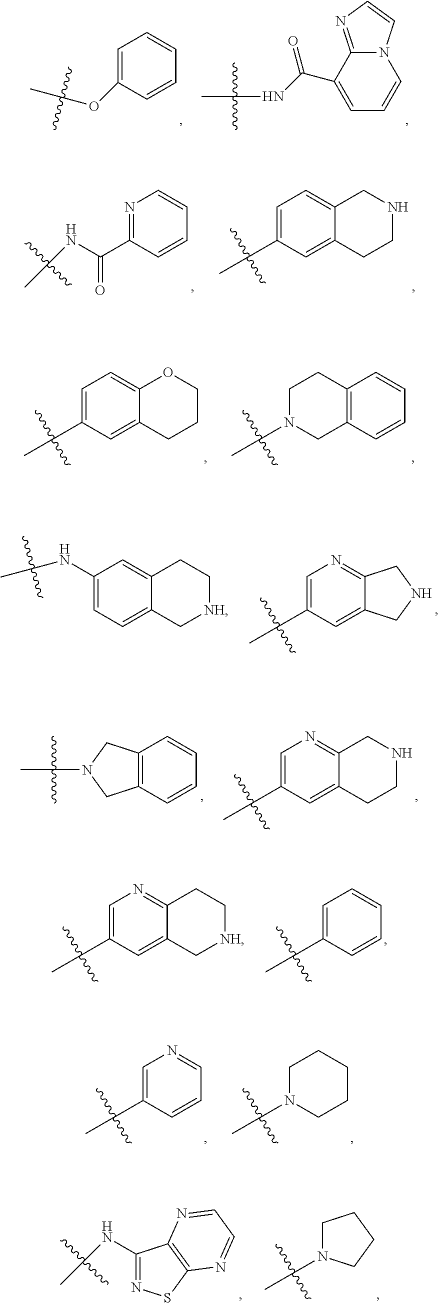 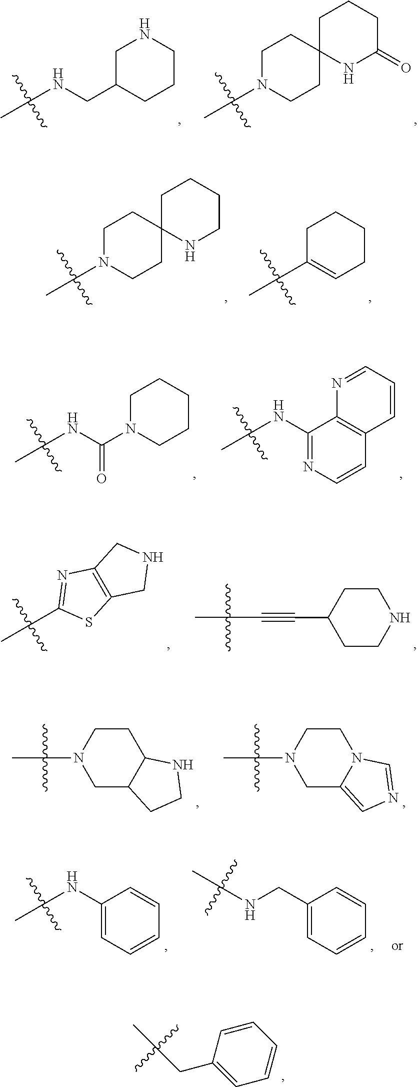
each of which is optionally substituted with 1, 2, 3, 4 or 5 independently selected R⁶ substituents.

27. The compound of claim 1, or a pharmaceutically acceptable salt or a stereoisomer thereof, wherein ring B: 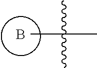
is selected from: 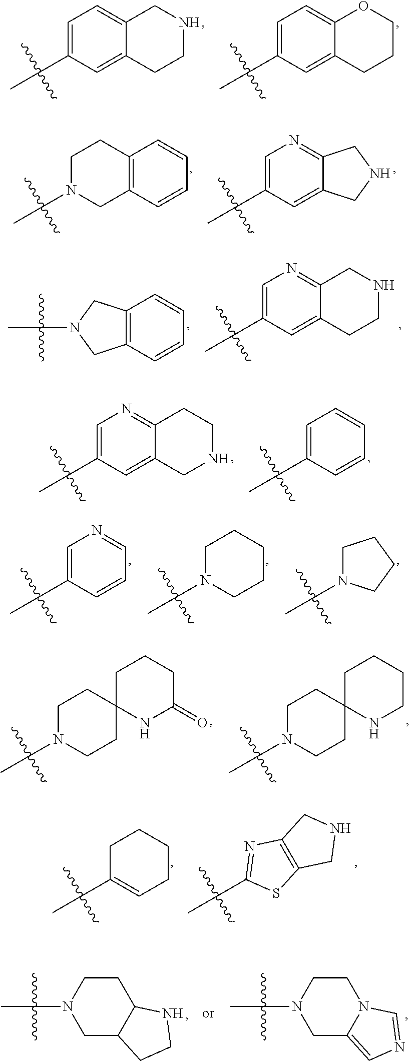
each of which is optionally substituted with 1 to 5 independently selected R⁵ substituents.

28. The compound of claim 1, or a pharmaceutically acceptable salt or a stereoisomer 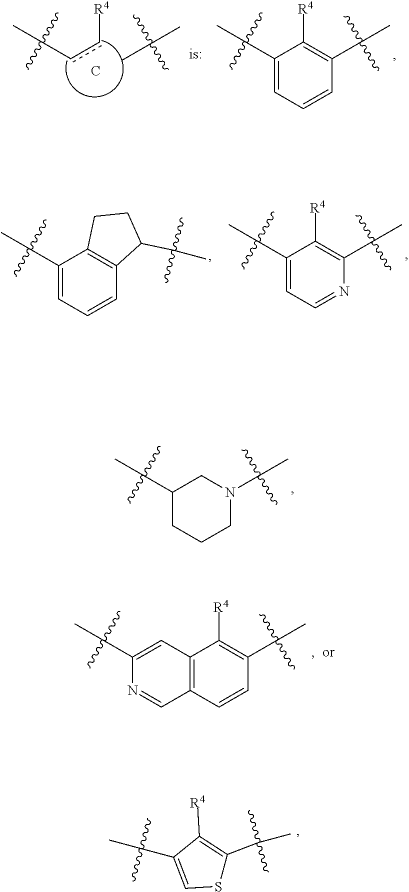
each of which is optionally substituted with 1, 2 or 3 independently selected R³¹ substituents.

29. The compound of claim 1, or a pharmaceutically acceptable salt or a stereoisomer thereof, wherein L¹ is a bond, —O—, —NHC(O)—, —NH—, —CH₂NH—, or —CH₂—.

30. The compound of claim 6, or a pharmaceutically acceptable salt or a stereoisomer thereof, wherein L² is a bond.

31. The compound of claim 1, or a pharmaceutically acceptable salt or a stereoisomer thereof, wherein L³ is a bond, —O—, —NHC(O)—, —NH—, —CH₂NH—, or —CH₂—.

32. A compound of Formula (XI): 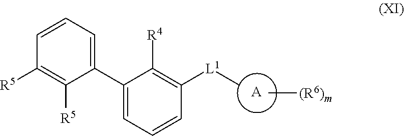
or a pharmaceutically acceptable salt or a stereoisomer thereof, wherein:
L¹ is a bond, O, —NR¹³—, or —CH═CH—;
ring A is 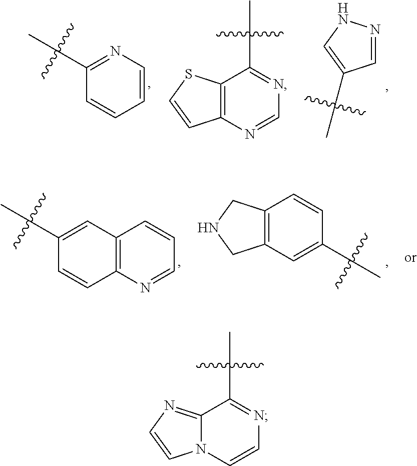
wherein [symbol] indicates the point of attachment of ring A to L¹;
each R¹³ is independently H, C₁₋₆ haloalkyl or C₁₋₆ alkyl;
R⁴ is halo or C₁₋₆ alkyl;
each R⁵ is independently selected from halo and OR^{a};
each R⁶ is independently selected from halo, C₁₋₆ alkyl, C₂₋₆ alkenyl, C₂₋₆ alkynyl, C₁₋₆ haloalkyl, C₁₋₆ haloalkoxy, 5-14 membered heteroaryl, 4-10 membered heterocycloalkyl, (5-14 membered heteroaryl)-C₁₋₄ alkyl-, (4-10 membered heterocycloalkyl)-C₁₋₄ alkyl-, CN, NO₂, OR^{a}, C(O)R^{a}, C(O)NR^{a}R^{a}, C(O)OR^{a}, NHR^{a}, NR^{a}R^{a}, NR^{a}C(O)R^{a}, and NR^{a}C(O)OR^{a}, wherein the C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, and (4-10 membered heterocycloalkyl)-C₁₋₄ alkyl- of R⁶ are each optionally substituted with 1, 2 or 3 independently selected R^{b} substituents;
each R^{a} is independently selected from H, C₁₋₆ alkyl, C₁₋₆ haloalkyl, C₂₋₆ alkenyl, and C₂₋₆ alkynyl;
each R^{b} substituent is independently selected from halo, C₁₋₆ alkyl, C₁₋₆ haloalkyl, C₁₋₆ haloalkoxy, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, CN, OH, NH₂, NO₂, OR^{c}, C(O)R^{c}, C(O)NR^{c}R^{c}, C(O)OR^{c}, NHR^{c}, NR^{c}R^{c}, NR^{c}C(O)R^{c}, and NR^{c}C(O)OR^{c};
each R^{c} is independently selected from H, C₁₋₆ alkyl, C₁₋₆ haloalkyl, C₂₋₆ alkenyl, and C₂₋₆ alkynyl, wherein the C₁₋₆ alkyl, C₁₋₆ haloalkyl, C₂₋₆ alkenyl, and C₂₋₆ alkynyl R^{c} are each optionally substituted with 1, 2 or 3 R^{f} substituents;
each R^{f} is independently selected from C₁₋₆ alkyl, C₁₋₆ haloalkyl, C₂₋₆ alkenyl, C₂₋₆ alkynyl, halo, CN, OR^{g}, C(O)R^{g}, C(O)NR^{g}R^{g}, C(O)OR^{g}, NHR^{g}, NR^{g}R^{g}, NR^{g}C(O)R^{g}, and NR^{g}C(O)OR^{g};
each R^{g} is independently selected from H, C₁₋₆ alkyl, C₁₋₆ haloalkyl, C₂₋₆ alkenyl, and C₂₋₆ alkynyl;
or any two R^{c} substituents together with the nitrogen atom to which they are attached form a 4-, 5-, 6-, or 7-membered heterocycloalkyl group optionally substituted with 1, 2, or 3 independently selected R^{h} substituents;
each R^{h} is independently selected from C₁₋₆ alkyl, C₁₋₆ haloalkyl, C₁₋₆ haloalkoxy, C₂₋₆ alkenyl, C₂₋₆ alkynyl, halo, CN, ORⁱ, C(O)Rⁱ, C(O)NRⁱRⁱ, C(O)ORⁱ, NHRⁱ, NRⁱRⁱ, NRⁱC(O)Rⁱ, and NRⁱC(O)ORⁱ;
each Rⁱ is independently selected from H, C₁₋₆ alkyl, and C₁₋₆ haloalkyl; and
the subscript m is an integer of 0, 1, 2, or 3.

33. The compound of claim 32, or a pharmaceutically acceptable salt or a stereoisomer thereof, wherein:
L¹ is a bond, O, —NH—, or —CH═CH—;
ring A is 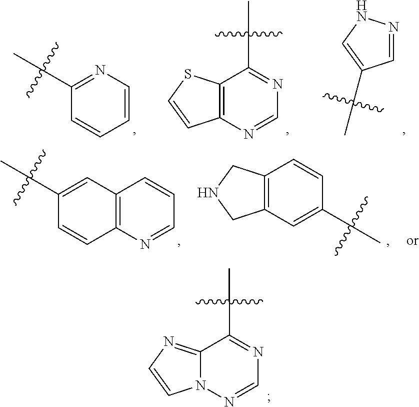
wherein [symbol] indicates the point of attachment of ring A to L¹;
R⁴ is halo or C₁₋₆ alkyl;
each R⁵ is independently selected from halo and OR^{a};
each R⁶ is independently selected from halo, C₁₋₆ alkyl, (4-10 membered heterocycloalkyl)-C₁₋₄ alkyl-, and OR^{a}, wherein the C₁₋₆ alkyl and (4-10 membered heterocycloalkyl)-C₁₋₄ alkyl- of R⁶ are each optionally substituted with 1, 2 or 3 independently selected R^{b} substituents;
each R^{a} is independently selected from H and C₁₋₆ alkyl;
each R^{b} substituent is independently selected from halo, C₁₋₆ alkyl, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, C(O)OR^{c}, NHR^{c}, and NR^{c}R^{c};
each R^{c} is independently selected from H and C₁₋₆ alkyl, wherein the C₁₋₆ alkyl is optionally substituted with 1 or 2 R^{f} substituents;
each R^{f} is independently selected from C₁₋₆ alkyl and OR^{g};
each R^{g} is independently selected from H and C₁₋₆ alkyl;
or any two R^{c} substituents together with the nitrogen atom to which they are attached form a 4-, 5-, 6-, or 7-membered heterocycloalkyl group optionally substituted with 1, 2, or 3 independently selected R^{h} substituents;
each R^{h} is C(O)ORⁱ;
each Rⁱ is independently selected from H and C₁₋₆ alkyl; and
the subscript m is an integer of 0, 1, 2, or 3.

34. The compound of claim 32, or a pharmaceutically acceptable salt or a stereoisomer thereof, wherein R⁴ is C₁₋₆ alkyl.

35. The compound of claim 32, or a pharmaceutically acceptable salt or a stereoisomer thereof, wherein R⁴ is halo.

36. The compound of claim 32, or a pharmaceutically acceptable salt or a stereoisomer thereof, wherein R⁵ is OR^{a}.

37. The compound of claim 32, or a pharmaceutically acceptable salt or a stereoisomer thereof, wherein R⁵ is halo.

38. The compound of claim 32, or a pharmaceutically acceptable salt or a stereoisomer thereof, wherein each R⁶ is independently selected from C₁₋₆ alkyl and (4-10 membered heterocycloalkyl)-C₁₋₄ alkyl-, wherein the C₁₋₆ alkyl and (4-10 membered heterocycloalkyl)-C₁₋₄ alkyl- of R⁶ are each optionally substituted with 1 or 2 independently selected R^{b} substituents.

39. The compound of claim 32, or a pharmaceutically acceptable salt or a stereoisomer thereof, wherein each R⁶ is 2-hydroxyethylaminomethyl, pyrrolidin-2-ylmethyl, methylpiperidine-2-carboxylic acid, or aminomethyl.

40. The compound of claim 32, or a pharmaceutically acceptable salt or a stereoisomer thereof, wherein ring A is

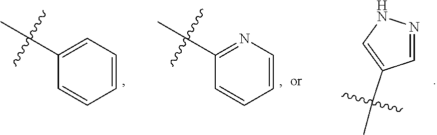 [SMILES: *c1ccccc1 ; *c1ccccn1 ; *c1cn[nH]c1]

41. The compound of claim 32, or a pharmaceutically acceptable salt or a stereoisomer thereof, wherein ring A is

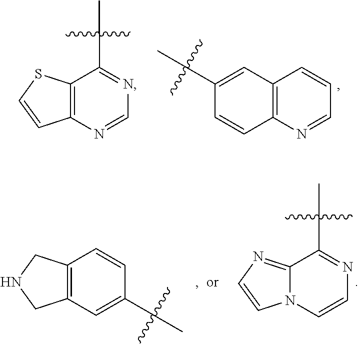 [SMILES: *c1ncnc2ccsc12 ; *c1ccc2ncccc2c1 ; *c1ccc2c(c1)CNC2 ; *c1nccn2ccnc12]

42. The compound of claim 1, selected from:
2-((4-(2-methylbiphenyl-3-yloxy)thieno[3,2-d]pyrimidin-7-yl)methylamino)ethanol;
4-(2-methylbiphenyl-3-yl)-1-[(2S)-pyrrolidin-2-ylmethyl]-1H-pyrazole;
2-((2-(2-chloro-2′-fluoro-3′-methoxybiphenyl-3-ylamino)-3-fluoropyridin-4-yl)methylamino)ethanol;
(S)-1-((4-methyl-6-(2-methylbiphenyl-3-yl)quinolin-2-yl)methyl)piperidine-2-carboxylic acid;
5-(2-methylbiphenyl-3-yl)isoindoline;
(5-(2-chlorobiphenyl-3-yl)pyridin-2-yl)methanamine;
2-{[(2′-methyl-1,1′:3′,1″-terphenyl-4-yl)methyl]amino}ethanol;
(R,E)-1-((6-(2-(2-methyl-[1,1′-biphenyl]-3-yl)vinyl)pyri din-3-yl)methyl)piperidine-2-carboxylic acid; and
2-(((8-((2′-fluoro-3′-methoxy-2-methyl-[1,1′-biphenyl]-3-yl)amino)imidazo[1,2-a]pyrazin-3-yl)methyl)amino)ethan-1-ol;
or a pharmaceutically acceptable salt or a stereoisomer thereof.

43. The compound of claim 1, selected from:
(2S)-1-(3-methyl-4-(4-phenyl-2,3-dihydro-1H-inden-1-yloxy)benzyl)piperidine-2-carboxylic acid;
(S)-1-((8-(2-methylbiphenyl-3-ylcarbamoyl)imidazo[1,2-a]pyridin-3-yl)methyl)piperidine-2-carboxylic acid;
N-(5-chloro-3-phenylisoquinolin-6-yl)-5-((2-hydroxyethylamino)methyl) picolinamide;
6-(2-methylbiphenyl-3-yl)-1,2,3,4-tetrahydroisoquinoline;
2-((2-(2-methylbiphenyl-3-yl)-1,2,3,4-tetrahydroisoquinolin-6-yl)methylamino)ethanol;
N-(2-methylbiphenyl-3-yl)-1,2,3,4-tetrahydroisoquinolin-6-amine;
N,N-dimethyl-1-(6-(2-methylbiphenyl-3-yl)chroman-2-yl)methanamine;
3-(2-methylbiphenyl-3-yl)-6,7-dihydro-5H-pyrrolo[3,4-b]pyridine;
2-(6-(2-methylbiphenyl-3-ylamino)-3,4-dihydroisoquinolin-2(1H)-yl)propane-1,3-diol;
2-((2-(2-methylbiphenyl-3-yl)isoindolin-5-yl)methylamino)ethanol;
3-(2-methylbiphenyl-3-yl)-5,6,7,8-tetrahydro-1,6-naphthyridine;
(4-(3-methyl-4-phenylpyridin-2-yl)phenyl)methanamine;
Cis-4-((5-(3-phenylpiperidin-1-yl)pyridin-2-yl)methylamino)cyclohexanol;
(1-((1-(2-chloro-3-(phenylamino)phenyl)piperidin-4-ylamino)methyl)cyclobutyl)methanol;
(1-((1-(3-(benzylamino)-2-chlorophenyl)piperidin-4-ylamino)methyl)cyclobutyl)methanol;
N-(2-methylbiphenyl-3-yl)isothiazolo[4,5-b]pyrazin-3-amine,
(R)-1-(2-methylbiphenyl-3-yl)piperidin-3-amine;
(1-(2-methylbiphenyl-3-yl)piperidin-4-yl)methanamine;
2-methyl-N-(piperidin-3-ylmethyl)biphenyl-3-amine,
(R)-(1-(2-methylbiphenyl-3-yl)pyrrolidin-3-yl)methanamine;
9-(2-methylbiphenyl-3-yl)-1,9-diazaspiro[5.5]undecan-2-one;
9-(2-methylbiphenyl-3-yl)-1,9-diazaspiro[5.5]undecane;
1-(4-(2-methylbiphenyl-3-yl)cyclohex-3-enyl)pyrrolidine;
3-amino-N-(2-methylbiphenyl-3-yl)piperidine-1-carboxamide;
2-[(3-{[(2-hydroxyethyl)amino]methyl}-1,7-naphthyridin-8-yl)amino]-4-phenylthiophene-3-carbonitrile;
(R)-2-(dimethylamino)-1-(2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazol-5 (6H)-yl)ethanone;
1-(2-(2,2′-dimethyl-3′-(piperidin-4-ylethynyl)biphenyl-3-yl)-4H-pyrrolo[3,4-d]thiazol-5 (6H)-yl)-2-(ethyl(methyl)amino)ethanone;
1-(2-(2,2′-dimethyl-3′-(tetrahydro-1H-pyrrolo[3,2-c]pyridin-5 (6H,7H,7aH)-yl)biphenyl-3-yl)-4H-pyrrolo[3,4-d]thiazol-5 (6H)-yl)-2-(ethyl(methyl)amino)ethanone;
1-(2-(3′-(5,6-dihydroimidazo[1,5-a]pyrazin-7 (8H)-yl)-2,2′-dimethylbiphenyl-3-yl)-4H-pyrrolo[3,4-d]thiazol-5 (6H)-yl)-2-(ethyl(methyl)amino)ethanone;
2-(2′-fluoro-3′-methoxy-2-methylbiphenyl-3-ylamino)-N-(2-(2-hydroxyethylamino)ethyl)nicotinamide; and
2-({3-chloro-4-[(4-phenyl-2,3-dihydro-1H-indol-1-yl)methyl]benzyl}amino)ethanol;
or a pharmaceutically acceptable salt or a stereoisomer thereof.

44. A pharmaceutical composition comprising a compound of claim 1, or a pharmaceutically acceptable salt or a stereoisomer thereof, and one or more pharmaceutically acceptable excipient or carrier.

45. A method of inhibiting PD-1/PD-L1 interaction, said method comprising administering to a patient a compound of claim 1, or a pharmaceutically acceptable salt or a stereoisomer thereof.

46. A method of treating a disease or disorder associated with inhibition of PD-1/PD-L1 interaction, said method comprising administering to a patient in need thereof a therapeutically effective amount of a compound of claim 1, or a pharmaceutically acceptable salt or a stereoisomer thereof.

47. A method of enhancing, stimulating and/or increasing the immune response in a patient, said method comprising administering to the patient in need thereof a therapeutically effective amount of a compound of claim 1, or a pharmaceutically acceptable salt or a stereoisomer thereof.
